SEQUENCE LISTING (139 sequences)

SEQ ID NO: 1 - DNA, 27 bp, Artificial sequence
  Query - pest seq (AF502391.1)
aaatgaagaaaatgcacagacctcaaa

SEQ ID NO: 2 - DNA, 27 bp, Artificial sequence
  Hit - plant seq (NM_001037071.1)
aaatgaagaaaatggaaagacctcaaa

SEQ ID NO: 3 - DNA, 102 bp, Artificial sequence
  Ath-MIR173 based GEiGS molecule
gaaggacuuuguaaacaauuucagaauuacugaggacaaaaauguuguaguacacuuaaagucgcaaaccgcggugauuugaauuuguuuguaaagaccuuc

SEQ ID NO: 4 - DNA, 1591 bp, Arabidopsis thaliana
acaccttccaaacactattggggaaatggcttcttctcttttttccggtgtaggtttaaggttttagatttgaaggcggatggtgaggagtttgtgttgatgaaatgggtgatttgaattgaggaaaacatgaattcgacatcgacacattttgtgccaccgagaagagttggtatatacgaacctgtccatcaattcggtatgtggggggagagtttcaaaagcaatattagcaatgggactatgaacacaccaaaccacataataataccgaataatcagaaactagacaacaacgtgtcagaggatacttcccatggaacagcaggaactcctcacatgttcgatcaagaagcttcaacgtctagacatcccgataagatacaaagacggcttgctcaaaaccgcgaggctgctaggaaaagtcgcttgcgcaagaaggcttatgttcagcaactggaaacaagcaggttgaagctaattcaattagagcaagaactcgatcgtgctagacaacagggattctatgtaggaaacggaatagatactaattctctcggtttttcggaaaccatgaatccagggattgctgcatttgaaatggaatatggacattgggttgaagaacagaacagacagatatgtgaactaagaacagttttacacggacacattaacgatatcgagcttcgttcgctagtcgaaaacgccatgaaacattactttgagcttttccggatgaaatcgtctgctgccaaagccgatgtcttcttcgtcatgtcagggatgtggagaacttcagcagaacgattcttcttatggattggcggatttcgaccctccgatcttctcaaggttcttttgccacattttgatgtcttgacggatcaacaacttctagatgtatgcaatctaaaacaatcgtgtcagcaagcagaagacgcgttgactcaaggtatggagaagctgcaacacacccttgcggactgcgttgcagcgggacaactcggtgaaggaagttacattcctcaggtgaattctgctatggatagattagaagctttggtcagtttcgtaaatcaggctgatcacttgagacatgaaacattgcaacaaatgtatcggatattgacaacgcgacaagcggctcgaggattattagctcttggtgagtattttcaacggcttagagccttgagctcaagttgggcaactcgacatcgtgaaccaacgtaggtttgagttattttgtaacaaccaaatgaagaaaatggaaagacctcaaaaaatgaagaatgagtgcatctgaaaacagaggactactctgaataaatagaggggttgctgctgatatttatttttactctgcggcggaattagaaaatttgaaaaacatcatgtattgataagttgtaaatatcagaaaaaggtgggggtgcaaaaatttgtactttttagcttttgaaagaggcaagtttttcgaatgtttgtttgatttgtaaacaatttcagaattatataaacttggttccaaatcccctgtaataatgtcgagctatctgcaatttgaaaactataggggctttactta

SEQ ID NO: 5 - DNA, 28 bp, Artificial sequence
  Query pest seq AF500024.1
cagcaacaacagaatcaggaacagcaac

SEQ ID NO: 6 - DNA, 28 bp, Artificial sequence
  Hit - plant seq (NM_116351.7)
cagcaacaacagcaacagcaacagcaac

SEQ ID NO: 7 - DNA, 123 bp, Artificial sequence
  Ath-MIR156a based GEiGS molecule
caagagaaacgcaaagauaaagauugaacugggguaucacacaaaggcaagaugcagaccagugcaguugcuucgcuugcgugaugcucaugguuuuaauuuuaauccggugccgaucucuuc

SEQ ID NO: 8 - DNA, 3676 bp, Arabidopsis thaliana
aaattaaattatgcgtctaagttgtaacatataataaaaggataatatatttttcagtcacaccaaaaaaaaaattgataagaaagaaaaaaaaagagtgaaaattagaaagacagagaagcaaagattcttgctccctgcgaatccgcagctgcttcgaaacgcaaatccgatttgtagtctcctttgattcttccattgacgatacagcttctctttctctcttctctctgcttactttgctttttactaagctcacaagaatctacgcatctgtactgttattatgctgatgctctctacttaatcatcaccaccgcacgcagttcgagatttctggaattttctgtgtcggatgcgtttgagattcatcgaatctacttggttatagattggaaatttaggtggggatttgagttgcttagcttgtggtaggttacattttcttgtttgagattcagtgaatctctccagagttgcatgtatagaaatgggttctttggaatctgggattccgacgaagcgagataacggcggcgtaagaggtggaagacagcaacaacagcaacagcaacagcaacagttcttcttgcagagaaacagatcgagactctccagattctttctattgaagagttttaattacctcctatggatttctataatttgtgtcttcttcttcttcgctgtgctgttccagatgtttttgccgggtttggtgattgataaatcggataaaccatggattagtaaggagattttgccacctgatttggttggttttagggagaaagggtttttggattttggtgatgatgttagaattgagcccaccaagcttctgatgaaattccaaagagatgctcatggttttaattttacatcttcttctctcaataccactctgcagcgttttggattcagaaagcctaagctagctctggtttttggcgatttgttagctgatccagaacaggtgttaatggtgtctctctccaaggcactgcaagaggttggctatgcaattgaggtttactcgcttgaagatggtccagtgaatagtatttggcagaaaatgggagttccagtcacaatactcaagcctaatcaggaatcgagttgtgttatcgactggctctcctatgatggcataattgtgaactctctccgagctaggagtatgtttacttgcttcatgcaagaacctttcaaatctttgcctcttatttgggtcatcaatgaagaaactcttgctgttcggtctagacagtacaactcaacagggcagactgaactcctcactgactggaaaaagattttcagccgggcatcggttgtagtcttccataattatctccttccgatactctacaccgagtttgatgctggcaacttctatgtgattcccggatctcctgaagaagtatgtaaagcaaagaatctagagtttcctccacagaaagatgatgtggtcatttccattgtgggaagtcagttcttgtacaagggtcaatggctggaacatgccctgcttctgcaagctctacggcctttattttcgggcaattaccttgaaagtgataattcccatctcaagatcatagttttaggtggagagacagcgtccaactacagcgtagctattgagacaatttcccagaacttgacatatccaaaagaggctgtgaagcacgtaagagttgcggggaatgttgataagattcttgaaagttctgatcttgttatatatggatcatttcttgaggagcagtcttttccagaaattttgatgaaggccatgtccttggggaaacctatagttgcaccagacctcttcaacattagaaaatatgttgacgacagggttactgggtatctcttccccaagcagaatcttaaagttctatcgcaagttgtgcttgaagtgataacagaagggaagatatctccattggctcagaagattgccatgatggggaaaacaactgttaaaaatatgatggctcgggaaaccatagaaggttatgcagctctactagagaatatgctcaagttttcttcggaagttgcttctcctaaggatgtacaaaaagttcctccagaactgagagaagagtggagttggcatccgtttgaagcttttatggatacatcgcctaataatagaatagcaagaagttatgagttcttagcgaaggttgaggggcattggaattataccccaggagaagctatgaaatttggagctgttaatgatgattcgttcgtgtatgaaatttgggaagaagagagatatcttcaaatgatgaatagtaaaaaaagacgagaagacgaggagctgaaaagcagagtcttgcagtatcgtgggacatgggaagatgtatataaaagcgccaaaagggcagaccgaagtaagaatgatctacatgagagggatgaaggggagctgctaagaaccggtcaacctttatgcatatatgaaccctattttggtgaaggaacctggtcgtttctacatcaagatcccctctatcgtggggttggcctgtcagttaaaggacgtagacctaggatggatgatgtcgatgcatcatcacgtcttccgcttttcaacaatccgtactatcgcgatgctcttggtgactttggagctttttttgcaatctcaaacaagattgatcggttacacaagaattcatggattgggtttcagtcctggagagcgactgccaggaaggaatctttatccaagattgctgaagacgcattacttaatgctatacaaacacgaaaacacggagatgccttatatttttgggttcgcatggacaaagatcccagaaatcctctgcagaaacccttttggtcgttctgtgatgccataaatgctgggaattgcaggtttgcttacaacgaaactttgaagaaaatgtacagtatcaagaacttggactcattgccaccaatgcccgaggatggggatacatggtctgtgatgcagagctgggcattgccaacaagatccttcttagagtttgtcatgttctcaaggatgtttgtggattcactagatgcacagatatatgaagagcatcatcgaacaaaccgttgctatctgagtttaaccaaagacaagcattgctattcgcgggtactagagcttctggtgaacgtatgggcttaccacagtgcaagacgcattgtctacatagatcctgagactggtttgatgcaagagcaacacaaacagaagaaccggcgagggaaaatgtgggtgaagtggttcgattacacaactctgaaaacaatggacgaagatctagctgaagaagccgactcagaccgtcgtgtgggtcactggctatggccatggactggcgagatcgtgtggcgcggtacattagagaaagagaagcaaaagaagaatttagagaaagaggagaagaagaagaagagtcgagataagctgagtagaatgagaagtagaagtggtcgtcagaaagtgatcggaaaatatgtaaaaccaccgcctgagaacgaaactgttaccggaaattccactttgttaaatgtagtagacgcataaaagaaaacaaattaaaattgcttctttttttgttaggtcactaatttgtttcattcattgtttttggaaagattactgtataaaaggtct

SEQ ID NO: 9 - DNA, 231 bp, Artificial sequence
  Pest Query AF469060.1
tggcatgcaaattttcgtgaagacattgacgggcaaaacgatcactttggaggtggagagctcggacactgtggacaatgtgaaggagaagatccaagagaaggagggcattccgccggatcagcaacggctgatcttcgccggcaaacagctcgaggacggacgaacgttggccgactacaacatacagaaggagtccacgctccacttggtcctccgtctccggggcgg

SEQ ID NO: 10 - DNA, 231 bp, Artificial sequence
  Plant hit NM_001203752.2
tggtatgcagattttcgttaaaaccctaacgggaaagacgattactcttgaggtggagagctctgacaccattgacaacgtcaaggccaagatccaagataaggagggcattcctccggaccagcagcgtctcatcttcgctggaaagcagcttgaggatggacgtactttggccgactacaacatccagaaggagtctactcttcacttggtcctccgtctccgtggtgg

SEQ ID NO: 11 - DNA, 104 bp, Artificial sequence
  Ath-MIR156c based GEiGS molecule
cgcacagaaagggugauaauggcuugguugcacuaaggcacguugcauggccgaugcauaugcuucucuagcgcaaccaaguucauguaucguuuauucccgca

SEQ ID NO: 12 - DNA, 901 bp, Arabidopsis thaliana
caaatctctcaaccgtgatcaaggtagatttctgagttcttattgtatttcttcgatttgtttcgttcgatcgcaatttaggctctgttctttgattttgatctcgttaatctctgatcggaggcaaattacatagtttcatcgttagatctcttcttatttctcgattaggatgcagatcttcgttaagactctcaccggaaagactatcaccctcgaggtggaaagctctgacaccatcgacaacgttaaggccaagatccaggataaggaaggtattcctccggatcagcagaggcttatcttcgccggaaagcagttggaggatggccgcacgttggcggattacaatatccagaaggaatccaccctccacttggttctcaggctccgtggtggtatgcagattttcgttaaaaccctaacgggaaagacgattactcttgaggtggagagctctgacaccattgacaacgtcaaggccaagatccaagataaggagggcattcctccggaccagcagcgtctcatcttcgctggaaagcagcttgaggatggacgtactttggccgactacaacatccagaaggagtctactcttcacttggtcctccgtctccgtggtggtttctaaaccttgtctctctctcttatggttactgaaccaagttcatgtatcgtttcatctagtactttggtggtttatgttttggggccatgtacagcctctgataaataattgatcgactatgtttccgtttctttcatctctcttttctttcaaacaacaaatcgaacttattctctattgcaattatctctttcgattcacttttgtcatcgttgtctctttatatgatgtgcttagtttgatgagtgtgagaagtacagagtctctatc

SEQ ID NO: 13 - DNA, 21 bp, Artificial sequence
  suggested small interfering RNAs nucleic acid sequence
cacagtaaaattgaacaaata

SEQ ID NO: 14 - DNA, 21 bp, Artificial sequence
  suggested small interfering RNAs nucleic acid sequence
cacagtaaaattgaacaaata

SEQ ID NO: 15 - DNA, 21 bp, Artificial sequence
  suggested small interfering RNAs nucleic acid sequence
cacagtaaaattgaacaaata

SEQ ID NO: 16 - DNA, 21 bp, Artificial sequence
  suggested small interfering RNAs nucleic acid sequence
cacagtaaaattgaacaaata

SEQ ID NO: 17 - DNA, 21 bp, Artificial sequence
  suggested small interfering RNAs nucleic acid sequence
cacagtaaaattgaacaaata

SEQ ID NO: 18 - DNA, 21 bp, Artificial sequence
  suggested small interfering RNAs nucleic acid sequence
ctgcgatggcatgcaaatttt

SEQ ID NO: 19 - DNA, 21 bp, Artificial sequence
  suggested small interfering RNAs nucleic acid sequence
ctgcgatggcatgcaaatttt

SEQ ID NO: 20 - DNA, 21 bp, Artificial sequence
  suggested small interfering RNAs nucleic acid sequence
ctgcgatggcatgcaaatttt

SEQ ID NO: 21 - DNA, 21 bp, Artificial sequence
  suggested small interfering RNAs nucleic acid sequence
ctgcgatggcatgcaaatttt

SEQ ID NO: 22 - DNA, 21 bp, Artificial sequence
  suggested small interfering RNAs nucleic acid sequence
ctgcgatggcatgcaaatttt

SEQ ID NO: 23 - DNA, 21 bp, Artificial sequence
  suggested small interfering RNAs nucleic acid sequence
taaaatggaaatagacaatat

SEQ ID NO: 24 - DNA, 21 bp, Artificial sequence
  suggested small interfering RNAs nucleic acid sequence
taaaatggaaatagacaatat

SEQ ID NO: 25 - DNA, 21 bp, Artificial sequence
  suggested small interfering RNAs nucleic acid sequence
taaaatggaaatagacaatat

SEQ ID NO: 26 - DNA, 21 bp, Artificial sequence
  suggested small interfering RNAs nucleic acid sequence
taaaatggaaatagacaatat

SEQ ID NO: 27 - DNA, 21 bp, Artificial sequence
  suggested small interfering RNAs nucleic acid sequence
taaaatggaaatagacaatat

SEQ ID NO: 28 - DNA, 21 bp, Artificial sequence
  suggested small interfering RNAs nucleic acid sequence
gagaaggaaaatacacaatta

SEQ ID NO: 29 - DNA, 21 bp, Artificial sequence
  suggested small interfering RNAs nucleic acid sequence
gagaaggaaaatacacaatta

SEQ ID NO: 30 - DNA, 21 bp, Artificial sequence
  suggested small interfering RNAs nucleic acid sequence
gagaaggaaaatacacaatta

SEQ ID NO: 31 - DNA, 21 bp, Artificial sequence
  suggested small interfering RNAs nucleic acid sequence
gagaaggaaaatacacaatta

SEQ ID NO: 32 - DNA, 21 bp, Artificial sequence
  suggested small interfering RNAs nucleic acid sequence
gagaaggaaaatacacaatta

SEQ ID NO: 33 - DNA, 21 bp, Artificial sequence
  suggested small interfering RNAs nucleic acid sequence
tagttaggaaatttcaaataa

SEQ ID NO: 34 - DNA, 21 bp, Artificial sequence
  suggested small interfering RNAs nucleic acid sequence
tagttaggaaatttcaaataa

SEQ ID NO: 35 - DNA, 21 bp, Artificial sequence
  suggested small interfering RNAs nucleic acid sequence
tagttaggaaatttcaaataa

SEQ ID NO: 36 - DNA, 21 bp, Artificial sequence
  suggested small interfering RNAs nucleic acid sequence
tagttaggaaatttcaaataa

SEQ ID NO: 37 - DNA, 21 bp, Artificial sequence
  suggested small interfering RNAs nucleic acid sequence
tagttaggaaatttcaaataa

SEQ ID NO: 38 - DNA, 21 bp, Artificial sequence
  suggested small interfering RNAs nucleic acid sequence
atgggaatatattaaaacttt

SEQ ID NO: 39 - DNA, 21 bp, Artificial sequence
  suggested small interfering RNAs nucleic acid sequence
atgggaatatattaaaacttt

SEQ ID NO: 40 - DNA, 21 bp, Artificial sequence
  suggested small interfering RNAs nucleic acid sequence
atgggaatatattaaaacttt

SEQ ID NO: 41 - DNA, 21 bp, Artificial sequence
  suggested small interfering RNAs nucleic acid sequence
atgggaatatattaaaacttt

SEQ ID NO: 42 - DNA, 21 bp, Artificial sequence
  suggested small interfering RNAs nucleic acid sequence
atgggaatatattaaaacttt

SEQ ID NO: 43 - DNA, 21 bp, Artificial sequence
  suggested small interfering RNAs nucleic acid sequence
tggagcaatcattctgaatga

SEQ ID NO: 44 - DNA, 21 bp, Artificial sequence
  suggested small interfering RNAs nucleic acid sequence
tggagcaatcattctgaatga

SEQ ID NO: 45 - DNA, 21 bp, Artificial sequence
  suggested small interfering RNAs nucleic acid sequence
ctcactccttttaaacaaata

SEQ ID NO: 46 - DNA, 21 bp, Artificial sequence
  suggested small interfering RNAs nucleic acid sequence
ctcactccttttaaacaaata

SEQ ID NO: 47 - DNA, 21 bp, Artificial sequence
  suggested small interfering RNAs nucleic acid sequence
atacatatagattgataacaa

SEQ ID NO: 48 - DNA, 21 bp, Artificial sequence
  suggested small interfering RNAs nucleic acid sequence
atacatatagattgataacaa

SEQ ID NO: 49 - DNA, 21 bp, Artificial sequence
  suggested small interfering RNAs nucleic acid sequence
ccaggattccatgtaaaaaaa

SEQ ID NO: 50 - DNA, 21 bp, Artificial sequence
  suggested small interfering RNAs nucleic acid sequence
ccaggattccatgtaaaaaaa

SEQ ID NO: 51 - DNA, 21 bp, Artificial sequence
  suggested small interfering RNAs nucleic acid sequence
caaccgcatgataaacgtgaa

SEQ ID NO: 52 - DNA, 21 bp, Artificial sequence
  suggested small interfering RNAs nucleic acid sequence
caaccgcatgataaacgtgaa

SEQ ID NO: 53 - DNA, 21 bp, Artificial sequence
  suggested small interfering RNAs nucleic acid sequence
ctgcatgttcttcatccccga

SEQ ID NO: 54 - DNA, 21 bp, Artificial sequence
  suggested small interfering RNAs nucleic acid sequence
ctgcatgttcttcatccccga

SEQ ID NO: 55 - DNA, 2397 bp, Meloidogyne incognita
atggcacaagagccgcccctgcaaattgtgatcccgaaattagacgaaggcaagacgatggcaatgcgtcggctgccgcctccggcacaaccggcggctgcggtgccgtccgccgaacgcgtgtccaatcgggacgaggacgcgcagaacaccgagccgtcaatgagcggcaagcaaatcgtcggattgatcatcccaccaccagacatccgaacgattgtggacaaaacggcccttttcgtcgctcgcaacggtttggaatttgagttgaaaatcaaagagcgcgaggcgtccaacatgcgcttcaacttcctcaaccccaccgacccgtactttgcctactaccggaataaggtcaacgaattcgagacgggcgtcgcttcagccgacacacaatctagcgtgaagatgccggaagccatccgtgaacacgtgaaacgggcagagttcattccacgacagccgcccaaaccgttcgaattctgtgccgaaccgtcaacgctgaacgccttcgatttagacctcatccatttgaccgctttgtttgttgcgcgaaacgggcgccaattcctcacacagctgatgaaccgcgaagtgcgtaactttcaattcgactttctgaagccgcagcactccaatttccaatacttcaccaagctggtcgagcagtacacaaaggtgctgatcccttccaaaaacattgtggacgagttgcgtgcacagctcagtcagcacaaaattgtggaggatgtgcgctatcgcgtcggttgggaccggcatcaaaaggcgctgaaggaccgcgaggaccaggcggtggagaaggagcgcatcgcgtacaaccaaatcgattggcacgagttcgtcgtggtccaaacggtggactttcagcccagcgaaacgctgaatttgccgcatttgtgcacgccaaaggacgtcggagcgcgcattttgctgcaacagcgcacggatgcggccaaagcggcggcggagagtgtggctatggaggtcgaatctgacgaggagggtggcggctcggagtcgggtggcgaggagacggacgaacgcggcgtcgccgaggcggacaacgcggcggttgagctgcgccagcacctgaacattgggacggagaagcagcacaccggctccacgctaacacaaccggcgcccgcagcgccgaacgttggcagcgttataatccgcgattacgaccccaaaaaggctcgcagcggtccagtggccaaaactaccgcctcctccgcggagaagtacatcatttcgccgctgaccaacgagcgcattccggcggacaaactgcacgagcatgtgcgctacaacactgtggacccgcagttcaaagagcaacgagaccgcgaacatatggatcgtcaggacgaggacttaggtatggcgcccggggcggagatcagccgtaacattgccaagttggccgaacggcggacggacatttttggcattggggagaagggtgtcgagcagacaatcattggcaaaaaattgggggaggaggagcgccaaatgccgcgttcggacccgaagaccatttgggacggccaacagtcgaccatcgacgcgaccactcgcgcagcccagcagagcgtgtcgttggagcagcagatcaatgaaattcaacgacagcacgggtacttgccgaaccccgctgcggaacgaatggcaccttcgatgccgccaacttcggcaccttcgcaacagtttggccaaccgccgacatcgacggcgatggctccgccgcaccgtcccccggctcattcgaccggcggtggaagcgttcaaaaaatcgtctctctgccgccccatcctcaacatcagcaaatgcgcccgccgatgccgccgcacttcatgccctcgggccagcgtccgcatttgccaccaccgcatatgggcatgccgccacacggcgttatgggaggaatacaaatgcctccgcatatgcaacctggcatgccgggaatgcccccgccggggatgatgcgtccgccccatggcgcctttatgcctccgcccccctctttcggcggcgatgagcctccgagcaaacgtccgcgtgaggagacgttggaatcggaagagcgttggctgcaaaaggttcgcggtcaaatcactgtgcaggtgtgcacgccacagaacgaggagtggaacttgaagggcgactccatccaagtcttgttggacatcagttcgtcggtaactgcacttaaatcgatgatccaagagcaaatcggcgttgctgcaggcaaacagaaattggtttatgagggtattttcatgaaagacaaccaaacgttggcctattacaactttatgccaaacgcggctgtccaacttcagctgaaggaacgtggaggccgaaagaaatga

SEQ ID NO: 56 - DNA, 789 bp, Heterodera glycines
atggcagtcggaaagaataagaaaatgggcaaaaagggagccaagaagaaggctgtcgatccgttcacacgcaaagaatggtacgacatcaaagcgccggcgatgttcacacatcgaaacgtcggcaaaacgttggtcaaccgtactcagggaacgcgcatttcgagcgactttctaaaaggccgcgtttacgaagtgtcactgggtgaccttaacagcactgacgccgactttcgaaagttccgcctgatctgtgaagaggtacagggcaagatttgcctgaccaactttcacggaatgtcgttcactcgggacaaactgtgctctattgtcaagaagtggcacacgctcattgaggcgaatgtggcagtgaagactaccgacggtttcatgctccgactcttttgtatcggctttaccaagcgaaatgccaatcaaattaagaagacgagctatgcaaaagcctctcaggtgcggatgattcgtgccaaaatggtggagatcatgcagaaagaggtctcttccggcgatctgaaggaagtagtcaacaagctgatcccggattcgatcggcaaagacatagagaaggcgtgctccttctactaccctctgcaggacgtttacattcgtaaggtgaaggtgctgaagaagccgaagttcgagctgggcaaactattggagatgcatggggagggtgccggaacggtcgctacgattacgacggccgccggtgaaaaaattgagagccgtccggatgcgtacgaaccgcctgttcaacagagcgtttga

SEQ ID NO: 57 - DNA, 2496 bp, Heterodera glycines
atggttggtattgatcaaacgactagtgatgaaattattcaagataaagagcagttaaagtggaagatggacaatttgcattggtttcctgaagatgagcttccgccgaacgactttccgcctgcgctaagtatggaaagtgagggaagctcattcaacaacaatgtgaacgcggaaatggacgaggaaatgatgggggaagatacaatgcaccatgaagacgatcagccggtattgggcagcgacgaggatgaacaggaggatccaaaagactacaagaaaggcggttaccaccctgtgcaaattggcgatgtcttcaaacatggacgataccatgtcatccgaaaattgggctggggccatttctccaccgtttggcttagttgggacatcgacgtgaaacgatttgtcgctatgaaaatagtcaaatcggctgagcattacacagaggcagcgttggacgaaataaaattgctcgaatgtgtgagggattctgatccagccgacgcttcacttcagagagttgtccaactgctcgatcatttcacagtcagcggcgtcaacggtgcccatgtttgtatggtttttgaggttctcgggtgcaatttgttgaagcttatcattcgtagcagctacgagggcttgccgataaacctggtcaaaaggataacgaaacaggttctcgaaggacttcactatttgcatgagaaatgtcatatcattcatacggacataaaacccgagaatgttttgatcaccatgagtcatgaagagatcaaaaagatggcggaagatgcgattttggcgggaaaggcggggaatgccatgtccggttctgctgtttgcagctcgaagagggccttcaagaagatggaggaaacgcttacaaagaacaaaaagaagaagctgaagaagaaacggaagagacatcgcgacattcttgaacagcagctcaaagaagttgagggaatgagtgtggaaattaccagcccaatcagtgaacagaatccatttcgcatcaaccaccaacggcacgatggcgtttattcctcggacgacaacgaggaggacggagagagttgtagggaaaacgacgctcaactgacaaagaatctctcacttttggagaaaatcaaaattccgcgcatttctttgacccaattttcaactaacaattcgcaccaaaacaacactaataacagcaataataataacaacgaacaacaacaacagcagcagcagcaacaacaactccaattgggggaggagggcacgaagaatgggaaaagggttgctgcgtctggcagaccatcgaagttggtcaattcgacccaaaaatcttgcgccagaagcgaaaatgatgagcaaaaaccggtcaaaaaggaaccgtcggtgacgggaaatgccgaaccgggccaatcgtcggacgcactgccgaaacgaattggcaaaaagaaaaagacgggcaaaaacaaacaaatgttggtcaaaaaggataagagagacgattcaccctcgcctcctatcaaaagggaggaagaggaggccatgggacccgaagaggacgacaccaaagagtcgccgacaaaggggaaaacggccaaactgtcgaatgagtcggacgatggcaaaacgatgggttatgatgggatgggaaaaggaggcgaagaagcagcggaaaatgtcaaagtgaatgcgcagcagcgagaggggcaggaagaagattcaatttcgaaggggaaaaagaagaaagcgaagcgaaagaataagaagaagctcaagcaacaagcgtatgcacaagaagaagatgaggagttgcgagaaatcgacaaaagtgatggtgttcaccatcaaagcatggtcgactcggggcagaacaaggagctaaaaacagaggaagattatcaacagctgagtccaatggagcaggagatgtcgttcgaacacgaaaaccacgaaaagcaaatgctcgacaaaataattgccaagaaatttgatgtgaaaattgcggacctgggcaatgcttgttggacctaccatcacttcaccgaggacatccagacgcgtcagtacagagctttggaagtcatcatcggcgcgggctacgatacgtctgccgacatttggagtgtcgcctgtatggcttttgaattggcgaccggcgactatttattcgagccgcacagcggaggcacttacagcagggacgaggaccatcttgcgcatgtaatcgagctgttgggaagcattccgccgaccgttttcaaaaagggcgagcattggcgcgagtttttccataagaatggtcgtctgcttcacataccgaacctgaagccttggtcactggtcgaagttcttacgcagaagtaccaatggcctttcgagcaagccagatcgttcgcggcatttttgtttcctatgcttaactatgaaccggctgaacgcgtcactgcagcacagtgtctaaagcataattggctcaaaaacatagaatga

SEQ ID NO: 58 - DNA, 2946 bp, Heterodera glycines
atgagttcttctggtgaacaaccctgttacgcgctgattcatgtcgcaaacgacgtcgaatttccgtctgaagggcaattaaaggacaaatttgagcacggggacacaaaatccaagacggatgcactgaaaaagctgattttgatgatccaggcgggcgagaaggtcaccagccagctaatgatgtacgtgatccgtttttgtctaccaacatccgatcattatctgaagaagttgctgctgatattctgggaggtcgtcccgaagacaaatcaagagggcaaactgttgcacgaaatgattttagtctgcgacgcgtaccgcaaagatttgcagcatccgaacgaatacatacgcggctcgaccttgcgattcctttgtaaactaaaagagcccgagttgctcgagccgttgatgccgtcgattcggaagtgcttggagcatcgccattcctacgtacgccgcaattccattttggcaatctacacaatttacaaaaatttcgagtttttgattcccgatgcgcccgaattgatccaacaactgctagagaccgagcaggacgcctcctgcaagcgcaacgcgttcattatgcttctacacgttgaccggcagcgggccttggattatttgtccggttgtattgagcaggttgcgcagttcggcgacatccttcagcttatcattgtggagctgatctacaaagtctgtcacaacaacccggcggaacgcaatcgcttcattcgttgtgtgtacaatttgctgcagtcgcagtcgggcgccgttcgctacgaggcggctggcacacttgtgacgcttagcaccgcgccgacggcggtgaaagcggccgcaaccgcttacattgagctgatcgtcaaagagtcggacaacaatgttaagctgattgtactcgatcgtctggtatgcctacgcgaggttttgcccaacgacaaagtgctgcaggatttggtaatggacattctgcgtgttctgtccacaacggactacgaagtgcgccgaaagatcctgcagttggcgcttgaactggtctcatcgcgcaacgttcatgagatggtgatgtttttgcgcaaggagatcgacaaaacaaacaacgacacgcaagaggacaccggccggtacagacagttgctggtccgcaccctgcacagtgcgacaatcaaattcccggacgtggccattcaaatcttcccagtgctaatggagtttttgtcagatcagaacgaaagtgcggcgttggatgtgttagtgtttgtgcgtgaggcgattcaacgactgccgcacctgcgccatcacatcaccaaccaattgctggaagtttttccgaccatccgaaacgcgtccatttttcgatccgctctgtggattcttggcgaatattgcgagaattctgaggcaattggtcgtttattcgttttggtcaagttgtcggtgggtgaattgcctattgtcgagtcggagagtcgggaccgagatggtggagcagcaaaggacgaggcatcggcacctagaaagagtggagtcgatggcaagaccagccagcagaaactgatcaccgcggatggcacttacgccaaacaatcggctatcttttctgccgcttcgaacgccgttagtgcggctgacgataaaccaattttgcgcagttttctactcgatgggaacttctttattgcgtcagcgttggccaacactttggcgaagttggtgcttcgttatgcggaactgaataaaggtgtagcgtcaaccgttaacaaattggcgagcgaagcgctgctgcttatcgcatccattattcacctcggcaagtccggcatgtgcaaacaggcgataactgaggacgatctggaccgtctctccaccacagtgcgtttgatcgtggaccaatggcctgatgcagtgcatgtattcctgaatgagtgtcgttcgtcacttgagcgcatgttaatggccaaaggtgatgtggaccggcacgagcgcgagaccaaggcgccgaagaaaaagattcctgacaagactatcatgttcacgcaactgtccacccgagtttcggagaatgtcacagataccaatcttttcgacctttcgttgtctcaagcgcttggcacggcacccaaaactaccaaatacaactttgctagttccaaacttggcaaagtgatccagctggccggcttctcagaccccgtctatgcggaagcgtacgtcaacgtcaaccagtacgacattgttttggatgtgctcgtggtcaaccaaaccagcgacacactgcagaatctgacgctggagctgtcgactgtgggcgacttgaaactggtggacaaaccatctccaattacattggcgcccaatgacttcaccaacatcaaagccaccgtcaaagtgtcgtccaccgaaaatggagtaattttttcgaccattgcttacgatgtgcgcggatcaacatcggatcggaactgtgtgtacctggaggacatccacattgacataatggattacattgtgccgggaacttgcactgacacggagtttcgcaaaatgtgggccgaatttgagtgggaaaacaaggttggcgttgtgacgccgatcacggaccttcgccagtatctggaccatttgtcggctcaaacaaacatgaagctgttgaccacggatgccgcattagagggcgactgcggttttttggcggccaacttttgtgcccactccatttttggtgaggacgcattggccaatgtttccattgagaaggcggacccgcttgacccgatgagtgccatcattggacacattcggatcagggcgaagtcccaggggatggcactttcattgggggacaagataaaccacgcgcaaaaggagcgcaaaccggtggagaggggtggggcaagagcggctatgaatgccgctgccgccgccgcaaaataa

SEQ ID NO: 59 - DNA, 30 bp, Artificial sequence
  siRNA target site
tggagcagcagatcaatgaaattcaacgac

SEQ ID NO: 60 - DNA, 30 bp, Artificial sequence
  siRNA target site
attcgtaaggtgaaggtgctgaagaagccg

SEQ ID NO: 61 - DNA, 30 bp, Artificial sequence
  siRNA target site
aaaaacaaacaaatgttggtcaaaaaggat

SEQ ID NO: 62 - DNA, 30 bp, Artificial sequence
  siRNA target site
ccgctctgtggattcttggcgaatattgcg

SEQ ID NO: 63 - DNA, 21 bp, Artificial sequence
  Single strand DNA oligonucleotide
accaatttgacccaaaaaggc

SEQ ID NO: 64 - DNA, 24 bp, Artificial sequence
  Single strand DNA oligonucleotide
gcagcagatcaatgaaattcaacg

SEQ ID NO: 65 - DNA, 20 bp, Artificial sequence
  Single strand DNA oligonucleotide
agccgctctgtggattcttg

SEQ ID NO: 66 - DNA, 20 bp, Artificial sequence
  Single strand DNA oligonucleotide
aaactcctcgcctcttggtg

SEQ ID NO: 67 - DNA, 22 bp, Artificial sequence
  Single strand DNA oligonucleotide
tcttcagcaccttcaccttacg

SEQ ID NO: 68 - DNA, 25 bp, Artificial sequence
  Single strand DNA oligonucleotide
tcctttttgaccaacatttgtttgt

SEQ ID NO: 69 - DNA, 21 bp, Artificial sequence
  Single strand DNA oligonucleotide
accaatttgacccaaaaaggc

SEQ ID NO: 70 - DNA, 26 bp, Artificial sequence
  Single strand DNA oligonucleotide
tggacttagaatatgctatgttggac

SEQ ID NO: 71 - DNA, 20 bp, Artificial sequence
  Single strand DNA oligonucleotide
aaactcctcgcctcttggtg

SEQ ID NO: 72 - DNA, 27 bp, Artificial sequence
  Single strand DNA oligonucleotide
tctatctctacctctaattcgttcgag

SEQ ID NO: 73 - DNA, 839 bp, Artificial sequence
  AtTAS1b_AT1G50055
aaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagaagaatattctaagtccaacatagcatattctaagtccaacatagcgtaaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactc

SEQ ID NO: 74 - DNA, 23 bp, Artificial sequence
  sgRNA_AtTAS1b (including PAM)
ggacttagaatatgctatgttgg

SEQ ID NO: 75 - DNA, 839 bp, Artificial sequence
  GEiGS-Splicing factor-transcript
aaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagaaggtcgttgaatttcattgatctgctgctccaagtccaacatagcgtaaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactc

SEQ ID NO: 76 - DNA, 30 bp, Artificial sequence
  Homologous region in the GEiGS design of GEiGS-Splicing factor-transcript
gtcgttgaatttcattgatctgctgctcca

SEQ ID NO: 77 - DNA, 1000 bp, Artificial sequence
  GEiGS-Splicing factor-DONOR
gttgttgttgctaaatgaatgatattaatccactacagttgtaaactacaaatatacaaagcagtaaaaatccatgtatgatgattaataagaagctttcgaatggatatagaaaaaacttgaatcattgagtgtgaaaacataagtaatgtataactttttttttgttatcaagttgtaaacaactgaatttagattttggtacataaaacaaaaataaaattacaaacttgaagacaatttaattacaaagtgaatgatgtatgtaattggcggtagacttcatgacacatcataaaaagactacacaactcccattacaaatcttgcaaaacaagaataccaacatctacttttttttgcatatcctaaaatatgtttcgttaacataaaaatattacaaatatcattccggacatctcccatttttttttacgtataatatcattccggatatctcccaattttttacgctatgttggacttggagcagcagatcaatgaaattcaacgaccttctaccatcctatcaatcataactactctgataatatggtgaatggttagataccgatgaatgactcattcgcttgttgagaaaaatcacaagaaaacaaagttgtctcctcctgaaaatagatctccgtattccgataaaaccaaatacagagagcacgggactggtcgaaactgacacatggctggtgacaaatagaccgggacatcgaacctaatatctcaaaacctcaatctcaatctagctatagaagctactcaagagaagtaaaagaaagtacctttctatgagacaagaccatgactcgatttaacagcggaacctgatctaagaacctatctcaacttttgtgttaagatggagttacagatcgtagttgcagagcttgacggcgctcatgtttactctttttgttaaatgacgtcaggcttagccgcttaggtttagatttatgaggtcggtaatgagtgacttgggtttata

SEQ ID NO: 78 - DNA, 30 bp, Artificial sequence; identical to SEQ ID NO: 59
  Homologous region in the GEiGS design of GEiGS-Splicing factor-DONOR

SEQ ID NO: 79 - DNA, 839 bp, Artificial sequence
  GEiGS-Y25-transcript
aaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagcgcaatattcgccaagaatccacagagcggctaagtccaacatagcgtaaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactc

SEQ ID NO: 80 - DNA, 30 bp, Artificial sequence
  Homologous region in the GEiGS design of GEiGS-Y25-transcript
cgcaatattcgccaagaatccacagagcgg

SEQ ID NO: 81 - DNA, 1000 bp, Artificial sequence
  Y25-DONOR
gttgttgttgctaaatgaatgatattaatccactacagttgtaaactacaaatatacaaagcagtaaaaatccatgtatgatgattaataagaagctttcgaatggatatagaaaaaacttgaatcattgagtgtgaaaacataagtaatgtataactttttttttgttatcaagttgtaaacaactgaatttagattttggtacataaaacaaaaataaaattacaaacttgaagacaatttaattacaaagtgaatgatgtatgtaattggcggtagacttcatgacacatcataaaaagactacacaactcccattacaaatcttgcaaaacaagaataccaacatctacttttttttgcatatcctaaaatatgtttcgttaacataaaaatattacaaatatcattccggacatctcccatttttttttacgtataatatcattccggatatctcccaattttttacgctatgttggacttagccgctctgtggattcttggcgaatattgcgctaccatcctatcaatcataactactctgataatatggtgaatggttagataccgatgaatgactcattcgcttgttgagaaaaatcacaagaaaacaaagttgtctcctcctgaaaatagatctccgtattccgataaaaccaaatacagagagcacgggactggtcgaaactgacacatggctggtgacaaatagaccgggacatcgaacctaatatctcaaaacctcaatctcaatctagctatagaagctactcaagagaagtaaaagaaagtacctttctatgagacaagaccatgactcgatttaacagcggaacctgatctaagaacctatctcaacttttgtgttaagatggagttacagatcgtagttgcagagcttgacggcgctcatgtttactctttttgttaaatgacgtcaggcttagccgcttaggtttagatttatgaggtcggtaatgagtgacttgggtttata

SEQ ID NO: 82 - DNA, 30 bp, Artificial sequence; identical to SEQ ID NO: 62
  Homologous region in the GEiGS design of Y25-DONOR

SEQ ID NO: 83 - DNA, 947 bp, Artificial sequence
  AtTAS3a_AT3G17185
atcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttct

SEQ ID NO: 84 - DNA, 23 bp, Artificial sequence
  sgRNA_AtTAS3a (including PAM)
aaatgactcgaacgaattagagg

SEQ ID NO: 85 - DNA, 947 bp, Artificial sequence
  GEiGS-Ribosomal protein 3a-transcript
atcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatcggcttcttcagcaccttcaccttacgaattttctcctaccttgtctatccctcctgagctaatctccacatatatcttttgtttgttattgatgtatggttgacataaattcaataaagaagttgacgtttttct

SEQ ID NO: 86 - DNA, 30 bp, Artificial sequence
  Homologous region in the GEiGS design of GEiGS-Ribosomal protein 3a-transcript
cggcttcttcagcaccttcaccttacgaat

SEQ ID NO: 87 - DNA, 1000 bp, Artificial sequence
  GEiGS- Ribosomal protein 3a -DONOR
tgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatcggcttcttcagcaccttcaccttacgaattttctcctaccttgtctatccctcctgagctaatctccacatatatcttttgtttgttattgatgtatggttgacataaattcaataaagaagttgacgtttttcttatttgatttttgttgttgttggttatattattgcaacaaaattaaagggggtaaggaaggtctcgctatcaaggggactggcaaaaggtaatgaataaggaaacgggcaaaaagaattatgcctttactctctcttttaaggctttggacaggaatttagttttgttttatgtgttgtgttgtttgtttgggtctgactgaccccaaagggcaaagccaaaccagagaagactcttattaaatattccctcagaatcatttattgcctctatctttatctctctctttctcacactcgtgactgtttctcaccttatgtatgtactagtaatagtttttaccactttcaacttttacaaatagcatttgtttctgtttaaa

SEQ ID NO: 88 - DNA, 30 bp, Artificial sequence; identical to SEQ ID NO: 86
  Homologous region in the GEiGS design of GEiGS-Ribosomal protein 3a -DONOR

SEQ ID NO: 89 - DNA, 947 bp, Artificial sequence
  GEiGS-Spliceosomal SR protein-transcript
atcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatcctttttgaccaacatttgtttgtttttattttctcctaccttgtctatccctcctgagctaatctccacatatatcttttgtttgttattgatgtatggttgacataaattcaataaagaagttgacgtttttct

SEQ ID NO: 90 - DNA, 30 bp, Artificial sequence
  Homologous region in the GEiGS design of GEiGS-Spliceosomal SR protein-transcript
atcctttttgaccaacatttgtttgttttt

SEQ ID NO: 91 - DNA, 1000 bp, Artificial sequence
  GEiGS-Spliceosomal SR protein-DONOR
tgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctctttttcttgaccttgtaaggccttttcttgaccttgtaagaccccatctctttctaaacgttttattattttctcgttttacagattctattctatctcttctcaatatagaatagatatcctttttgaccaacatttgtttgtttttattttctcctaccttgtctatccctcctgagctaatctccacatatatcttttgtttgttattgatgtatggttgacataaattcaataaagaagttgacgtttttcttatttgatttttgttgttgttggttatattattgcaacaaaattaaagggggtaaggaaggtctcgctatcaaggggactggcaaaaggtaatgaataaggaaacgggcaaaaagaattatgcctttactctctcttttaaggctttggacaggaatttagttttgttttatgtgttgtgttgtttgtttgggtctgactgaccccaaagggcaaagccaaaccagagaagactcttattaaatattccctcagaatcatttattgcctctatctttatctctctctttctcacactcgtgactgtttctcaccttatgtatgtactagtaatagtttttaccactttcaacttttacaaatagcatttgtttctgtttaaa

SEQ ID NO: 92 - DNA, 30 bp, Artificial sequence; identical to SEQ ID NO: 90
  Homologous region in the GEiGS design of GEiGS-Spliceosomal SR protein-DONOR

SEQ ID NO: 93 - DNA, 30 bp, Artificial sequence
  Single strand DNA oligonucleotide
taacataaaaatattacaaatatcattccg

SEQ ID NO: 94 - DNA, 27 bp, Artificial sequence
  Single strand DNA oligonucleotide
tcagagtagttatgattgataggatgg

SEQ ID NO: 95 - DNA, 21 bp, Artificial sequence
  Single strand DNA oligonucleotide
gctcaggagggatagacaagg

SEQ ID NO: 96 - DNA, 27 bp, Artificial sequence
  Single strand DNA oligonucleotide
ctcgttttacagattctattctatctc

SEQ ID NO: 97 - DNA, 22 bp, Artificial sequence
  Single strand DNA oligonucleotide
tgaccttgtaagaccccatctc

SEQ ID NO: 98 - DNA, 24 bp, Artificial sequence
  Single strand DNA oligonucleotide
aggagaaaattcgtaaggtgaagg

SEQ ID NO: 99 - DNA, 22 bp, Artificial sequence
  Single strand DNA oligonucleotide
tgaccttgtaagaccccatctc

SEQ ID NO: 100 - DNA, 23 bp, Artificial sequence
  Single strand DNA oligonucleotide
ggtaggagaaaatgactcgaacg

SEQ ID NO: 101 - DNA, 27 bp, Artificial sequence
  Single strand DNA oligonucleotide
caaccatacatcaataacaaacaaaag

SEQ ID NO: 102 - DNA, 27 bp, Artificial sequence
  Single strand DNA oligonucleotide
atatagaatagatatcggcttcttcag

SEQ ID NO: 103 - DNA, 27 bp, Artificial sequence
  Single strand DNA oligonucleotide
caaccatacatcaataacaaacaaaag

SEQ ID NO: 104 - DNA, 25 bp, Artificial sequence
  Single strand DNA oligonucleotide
tcctttttgaccaacatttgtttgt

SEQ ID NO: 105 - DNA, 23 bp, Artificial sequence
  Single strand DNA oligonucleotide
gagtcattcatcggtatctaacc

SEQ ID NO: 106 - DNA, 20 bp, Artificial sequence
  Single strand DNA oligonucleotide
agccgctctgtggattcttg

SEQ ID NO: 107 - DNA, 23 bp, Artificial sequence
  Single strand DNA oligonucleotide
gagtcattcatcggtatctaacc

SEQ ID NO: 108 - DNA, 26 bp, Artificial sequence
  Single strand DNA oligonucleotide
tggacttagaatatgctatgttggac

SEQ ID NO: 109 - DNA, 27 bp, Artificial sequence
  Single strand DNA oligonucleotide
gcatatcctaaaatatgtttcgttaac

SEQ ID NO: 110 - DNA, 20 bp, Artificial sequence
  Single strand DNA oligonucleotide
tcgccaagaatccacagagc

SEQ ID NO: 111 - DNA, 27 bp, Artificial sequence
  Single strand DNA oligonucleotide
gcatatcctaaaatatgtttcgttaac

SEQ ID NO: 112 - DNA, 27 bp, Artificial sequence
  Single strand DNA oligonucleotide
taagtccaacatagcatattctaagtc

SEQ ID NO: 113 - DNA, 40 bp, Artificial sequence
  Single strand DNA oligonucleotide
atgtttgaacgatcgggcccaagggacacgaagtgatccg

SEQ ID NO: 114 - DNA, 38 bp, Artificial sequence
  Single strand DNA oligonucleotide
ctccaccatgttcccgggggcacagagtgttcaacccc

SEQ ID NO: 115 - DNA, 1038 bp, Artificial sequence
  AtTAS1B (At1g50055)
ttgacccaaaaaggctttgtactctttacataacaaaaaaggtcaaatagggaaaacgatgggactttataaacccaagtcactcattaccgacctcataaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagaagaatattctaagtccaacatagcatattctaagtccaacatagcgtaaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactcaatgattcaagttttttctatatccattcgaaagcttcttattaatcatcatacatggatttttactgctttgtatatttgtagtttacaactgtagtgg

SEQ ID NO: 116 - DNA, 1039 bp, Artificial sequence
  GEiGS-TuMV
tttgacccaaaaaggctttgtactctttacataacaaaaaaggtcaaatagggaaaacgatgggactttataaacccaagtcactcattaccgacctcataaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagaagaatattctaagtccaacatagcataacttgctcacacactcgactgaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactcaatgattcaagttttttctatatccattcgaaagcttcttattaatcatcatacatggatttttactgctttgtatatttgtagtttacaactgtagtgg

SEQ ID NO: 117 - DNA, 21 bp, Artificial sequence
  GEiGS-TuMV- mature siRNA
acttgctcacacactcgactg

SEQ ID NO: 118 - DNA, 1039 bp, Artificial sequence
  GEiGS-dummy
tttgacccaaaaaggctttgtactctttacataacaaaaaaggtcaaatagggaaaacgatgggactttataaacccaagtcactcattaccgacctcataaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagaagaatattggaggggtaatgccattgcttctaagtccaacatagcgtaaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactcaatgattcaagttttttctatatccattcgaaagcttcttattaatcatcatacatggatttttactgctttgtatatttgtagtttacaactgtagtgg

SEQ ID NO: 119 - DNA, 21 bp, Artificial sequence
  GEiGS-dummy- mature siRNA
ttggaggggtaatgccattgc

SEQ ID NO: 120 - DNA, 102 bp, Artificial sequence
  miR173_ AT3G23125
taagtactttcgcttgcagagagaaatcacagtggtcaaaaaagttgtagttttcttaaagtctctttcctctgtgattctctgtgtaagcgaaagagcttg

SEQ ID NO: 121 - DNA, 22 bp, Artificial sequence
  miR173-mature miRNA
ttcgcttgcagagagaaatcac

SEQ ID NO: 122 - DNA, 947 bp, Artificial sequence; identical to SEQ ID NO: 83
  AtTAS3a_AT3G17185

SEQ ID NO: 123 - DNA, 947 bp, Artificial sequence; identical to SEQ ID NO: 85
  GEiGS-Ribosomal protein 3a-transcript

SEQ ID NO: 124 - DNA, 30 bp, Artificial sequence; identical to SEQ ID NO: 86
  GEiGS-Ribosomal protein 3a-transcript - the expected processed siRNA

SEQ ID NO: 125 - DNA, 947 bp, Artificial sequence; identical to SEQ ID NO: 89
  GEiGS-Spliceosomal SR protein-transcript

SEQ ID NO: 126 - DNA, 30 bp, Artificial sequence; identical to SEQ ID NO: 90
  GEiGS-Spliceosomal SR protein-transcript - the expected processed siRNA

SEQ ID NO: 127 - DNA, 107 bp, Artificial sequence
  miR390_ AT2G38325
gtagagaagaatctgtaaagctcaggagggatagcgccatgatgatcacattcgttatctattttttggcgctatccatcctgagtttcattggctcttcttactac

SEQ ID NO: 128 - DNA, 25 bp, Artificial sequence
  Single strand DNA oligonucleotide
gctcaactgacaaagaatctctcac

SEQ ID NO: 129 - DNA, 25 bp, Artificial sequence
  Single strand DNA oligonucleotide
ttgaaaattgggtcaaagaaatgcg

SEQ ID NO: 130 - DNA, 21 bp, Artificial sequence
  Single strand DNA oligonucleotide
gaacggtcgctacgattacga

SEQ ID NO: 131 - DNA, 21 bp, Artificial sequence
  Single strand DNA oligonucleotide
caaacgctctgttgaacaggc

SEQ ID NO: 132 - DNA, 20 bp, Artificial sequence
  Single strand DNA oligonucleotide
ttccagcagatgtggatcag

SEQ ID NO: 133 - DNA, 20 bp, Artificial sequence
  Single strand DNA oligonucleotide
cggccttattcttcaagcac

SEQ ID NO: 134 - DNA, 23 bp, Artificial sequence
  sgRNA which would have been used to cut TAS1b
ccaacatagcgtaaaaaattggg

SEQ ID NO: 135 - DNA, 21 bp, Artificial sequence
  siRNA sequence that targets TuMV (which would have been introduced into the TAS1b backbone by the GEiGS donor using an HDR-mediated swap)
acttgctcacacactcgactg

SEQ ID NO: 136 - DNA, 1200 bp, Artificial sequence
  GEiGS donor which included the desired changes to the TAS1b backbone
atacataccaatttgacccaaaaaggctttgtactctttacataacaaaaaaggtcaaatagggaaaacgatgggactttataaacccaagtcactcattaccgacctcataaatctaaacctaagcggctaagcctgacgtcatttaacaaaaagagtaaacatgagcgccgtcaagctctgcaactacgatctgtaactccatcttaacacaaaagttgagataggttcttagatcaggttccgctgttaaatcgagtcatggtcttgtctcatagaaaggtactttcttttacttctcttgagtagcttctatagctagattgagattgaggttttgagatattaggttcgatgtcccggtctatttgtcaccagccatgtgtcagtttcgaccagtcccgtgctctctgtatttggttttatcggaatacggagatctattttcaggaggagacaactttgttttcttgtgatttttctcaacaagcgaatgagtcattcatcggtatctaaccattcaccatattatcagagtagttatgattgataggatggtagaagaatattctaagtccaacatagcataacttgctcacacactcgactgaaaaattgggagatatccggaatgatattatacgtaaaaaaaaatgggagatgtccggaatgatatttgtaatatttttatgttaacgaaacatattttaggatatgcaaaaaaaagtagatgttggtattcttgttttgcaagatttgtaatgggagttgtgtagtctttttatgatgtgtcatgaagtctaccgccaattacatacatcattcactttgtaattaaattgtcttcaagtttgtaattttatttttgttttatgtaccaaaatctaaattcagttgtttacaacttgataacaaaaaaaaagttatacattacttatgttttcacactcaatgattcaagttttttctatatccattcgaaagcttcttattaatcatcatacatggatttttactgctttgtatatttgtagtttacaactgtagtggattaatatcattcatttagcaacaacaacactcgttaagtttgctcacttgtcataattataaaccaacgcatgcatcacatatataagtaaatgcaaaacctatgcagctattatcaaagtcttcacttctcaaacgtgcttcaatcac

SEQ ID NO: 137 - DNA, 1039 bp, Artificial sequence; identical to SEQ ID NO: 116
  GEiGS oligo which would have been expressed in Arabidopsis following GEiGS with the donor of (3) (designed to introduce the mature siRNA sequence of (1) into the TAS1b sequence)

SEQ ID NO: 138 - DNA, 25 bp, Artificial sequence
  parameters for FASTQ
  misc_feature (1)..(4): n is a, c, g, t or u
nnnntggaattctcgggtgccaagg

SEQ ID NO: 139 - DNA, 67 bp, Artificial sequence
  parameters for FASTQ
agatcggaagagcacacgtctgaactccagtcaaagatcggaagagcgtcgtgtagggaaagagtgt

(19) United States
(12) Patent Application Publication
(10) Pub. No.: US 2022/0220494 A1
(43) Pub. Date: Jul. 14, 2022

(54) PRODUCTION OF dsRNA IN PLANT CELLS FOR PEST PROTECTION VIA GENE SILENCING

(71) Applicant: Tropic Biosciences UK Limited, Colney, Norwich (GB)

(72) Inventors: Eyal MAORI, Cambridge (GB); Yaron GALANTY, Coton, Cambridge (GB); Cristina PIGNOCCHI, Hethersett, Norwich (GB); Angela CHAPARRO GARCIA, Norwich (GB); Ofir MEIR, Norwich, Norfolk (GB)

(73) Assignee: Tropic Biosciences UK Limited, Colney, Norwich (GB)

(21) Appl. No.: 17/439,344

(22) PCT Filed: Mar. 14, 2020
(86) PCT No.: PCT/IB2020/052245

§ 371 Date: Sep. 14, 2021

(30) Foreign Application Priority Data
Mar. 14, 2019 (GB) 1903521.1

(51) Int. Cl. C12N 15/82; C12N 15/11; C12N 9/22

(52) U.S. Cl. CPC C12N 15/8218; C12N 15/8283; C12N 2310/20; C12N 15/111; C12N 9/22; C12N 15/8285

(57) ABSTRACT

A method of producing a long dsRNA molecule in a plant cell that is capable of silencing a pest gene is provided, the method comprising: (a) selecting m a genome of a plant a nucleic acid sequence encoding a silencing molecule having a plant gene as a target, the silencing molecule capable of recruiting RNA-dependent RNA Polymerase (RdRp); and (b) modifying a nucleic acid sequence of the plant gene so as to impart a silencing specificity towards the pest gene, such that a transcript of the plant gene comprising the silencing specificity forms base complementation with said silencing molecule capable of recruiting said RdRp to produce the long dsRNA molecule capable of silencing the pest gene, thereby producing the long dsRNA molecule m the plant cell that is capable of silencing the pest gene.

### RELATED APPLICATION/S

This application claims the benefit of priority of UK Patent Application No. 1903521.1 filed on 14 Mar. 2019, the contents of which are incorporated herein by reference in their entirety.

### SEQUENCE LISTING STATEMENT

The ASCII file, entitled 81321 Sequence Listing.txt, created on 12 Mar. 2020, comprising 73,728 bytes, submitted concurrently with the filing of this application is incorporated herein by reference.

### FIELD AND BACKGROUND OF THE INVENTION

The present invention, in some embodiments thereof, relates to generation and amplification of dsRNA molecules in a host cell for silencing pest target genes.

Recent advances in genome editing techniques have made it possible to alter DNA sequences in living cells by editing only a few of the billions of nucleotides in their genome. In the past decade, the tools and expertise for using genome editing, such as in human somatic cells and pluripotent cells, have increased to such an extent that the approach is now being developed widely as a strategy to treat human disease. The fundamental process depends on creating a site-specific DNA double-strand break (DSB) in the genome and then allowing the cell's endogenous DSB repair machinery to fix the break (such as by non-homologous end-joining (NHEJ) or homologous recombination (HR) in which the latter can allow precise one or more nucleotide changes to be made to the DNA sequence using exogenously provided donor template [Porteus, Annu Rev Pharmacol Toxicol. (2016) 56:163-90].

Three primary approaches use mutagenic genome editing (NHEJ) of cells, such as for potential therapeutics: (a) knocking out functional genetic elements by creating spatially precise insertions or deletions, (b) creating insertions or deletions that compensate for underlying frameshift mutations; hence reactivating partly- or non-functional genes, and (c) creating defined genetic deletions. Although several different applications use editing by NHEJ, the broadest applications of editing will probably harness genome editing by homologous recombination (HR), although a rare event it is highly accurate as it relies on an exogenously provided template to copy the correct sequence during the repair process.

Currently the four major types of applications to HR-mediated genome editing are: (a) gene correction (i.e. correction of diseases that are caused by point mutations in single genes), (b) functional gene correction (i.e. correction of diseases that are caused by point mutations scattered throughout the gene), (c) safe harbor gene addition (i.e. when precise regulation is not required or when supra non-physiological levels of a transgene are desired), and (d) targeted transgene addition (i.e. when precise regulation is required) [Porteus (2016), supra].

Previous work on genome editing of RNA molecules in various eukaryotic organisms (e.g. murine, human, shrimp, plants), focused on knocking-out miRNA gene activity or changing their binding site in target RNAs, for example:

With regard to genome editing in human cells, Jiang et al. [Jiang et al., RNA Biology (2014) 11 (10): 1243-9] used CRISPR/Cas9 to deplete human miR-93 from a cluster by targeting its 5′ region in HeLa cells. Various small were induced in the targeted region containing the Drosha processing site (i.e. the position at which Drosha, a double-stranded RNA-specific RNase III enzyme, binds, cleaves and thereby processes primary miRNAs (pri-miRNAs) into pre-miRNA in the nucleus of a host cell) and seed sequences (i.e. the conserved heptametrical sequences which are essential for the binding of the miRNA to mRNA, typically situated at positions 2-7 from the miRNA 5′-end). According to Jiang et al. even a single nucleotide deletion led to complete knockout of the target miRNA with high specificity.

With regard to genome editing in murine species, Zhao et al. [Zhao et al., Scientific Reports (2014) 4:3943] provided a miRNA inhibition strategy employing the CRISPR-Cas9 system in murine cells. Zhao used specifically designed sgRNAs to cut the miRNA gene at a single site by the Cas9 nuclease, resulting in knockout of the miRNA in these cells.

With regard to plant genome editing, Bortesi and Fischer [Bortesi and Fischer, Biotechnology Advances (2015) 33: 41-52] discussed the use of CRISPR-Cas9 technology in plants as compared to ZFNs and TALENs, and Basak and Nithin [Basak and Nithin, Front Plant Sci. (2015) 6: 1001] teach that CRISPR-Cas9 technology has been applied for knockdown of protein-coding genes in model plants such as Arabidopsis and tobacco and crops including wheat, maize, and rice.

In addition to disruption of miRNA activity or target binding sites, gene silencing using artificial miRNAs (amiRNAs) mediated gene silencing of endogenous and exogenous target genes has been achieved [Tiwari et al. Plant Mol Biol (2014) 86: 1]. Similar to miRNAs, amiRNAs are single-stranded, approximately 21 nucleotides (nt) long, and designed by replacing the mature miRNA sequences of the duplex within pre-miRNAs [Tiwari et al. (2014) supra]. These amiRNAs are introduced as a transgene within an artificial expression cassette (including a promoter, terminator etc.) [Carbonell et al., Plant Physiology (2014) pp.113.234989], are processed via small RNA biogenesis and silencing machinery and downregulate target expression. According to Schwab et al, [Schwab et al, The Plant Cell (2006) Vol. 18, 1121-1133], amiRNAs are active when expressed under tissue-specific or inducible promoters and can be used for specific gene silencing in plants, especially when several related, but not identical, target genes need to be downregulated.

Senis et al. [Senis et al., Nucleic Acids Research (2017) Vol. 45(1): e3] disclose engineering of a promoterless anti-viral RNAi hairpin into an endogenous miRNA locus. Specifically, Senis et al, insert an amiRNA precursor transgene (hairpin pri-amiRNA) adjacent to a naturally occurring miRNA gene (e.g. miR122) by homology-directed DNA recombination that is induced by sequence-specific nuclease such as Cas9 or TALEN nucleases. This approach uses promoter- and terminator-free amiRNAs by utilizing transcriptionally active DNA locus that expresses a natural miRNA (miR122), that is, the endogenous promoter and terminator drove and regulated the transcription of the inserted amiRNA transgene.

Various DNA-free methods of introducing RNA and/or proteins into cells have been previously described. For example, RNA transfection using electroporation and lipofection has been described in U.S. Patent Application No. 20160289675. Direct delivery of Cas9/sgRNA ribonucleoprotein (RNPs) complexes to cells by microinjection of the Cas9 protein and sgRNA complexes was described by Cho [Cho et al., “Heritable gene knockout in Caenorhabditis elegans by direct injection of Cas9-sgRNA ribonucleoproteins,” Genetics (2013) 195:1177-1180]. Delivery of Cas9 protein/sgRNA complexes via electroporation was described by Kim [Kim et al., “Highly efficient RNA-guided genome editing in human cells via delivery of purified Cas9 ribonucleoproteins” Genome Res. (2014) 24:1012-1019]. Delivery of Cas9 protein-associated sgRNA complexes via liposomes was reported by Zuris [Zuris et al., “Cationic lipid-mediated delivery of proteins enables efficient protein-based genome editing in vitro and in vivo” Nat Biotechnol. (2014) doi: 10. 1038/nbt.3081].

### SUMMARY OF THE INVENTION

According to an aspect of some embodiments of the present invention there is provided a method of producing a long dsRNA. molecule in a plant cell that is capable of silencing a pest gene, the method comprising: (a) selecting in a genome of a plant a nucleic acid sequence encoding a silencing molecule having a plant gene as a target, the silencing molecule capable of recruiting RNA-dependent RNA Polymerase (RdRp); and (b) modifying a nucleic acid sequence of the plant gene so as to impart a silencing specificity towards the pest gene, such that a transcript of the plant gene comprising the silencing specificity forms base complementation with the silencing molecule capable of recruiting the RdRp to produce the long dsRNA molecule capable of silencing the pest gene, thereby producing the long dsRNA molecule in the plant cell that is capable of silencing the pest gene.

According to an aspect of some embodiments of the present invention there is provided a method of producing a long dsRNA molecule in a plant cell that is capable of silencing a pest gene in a plant cell, the method comprising: (a) selecting in a genome of a plant a nucleic acid sequence encoding a silencing molecule having a plant gene as a target, the silencing molecule capable of recruiting RNA-dependent RNA Polymerase (RdRp); (b) modifying a nucleic acid sequence of the plant gene so as to impart a silencing specificity towards the pest gene, such that a transcript of the plant gene comprising the silencing specificity forms base complementation with the silencing molecule capable of recruiting the RdRp to produce the long dsRNA molecule capable of silencing the pest gene, thereby producing the long dsRNA molecule in the plant cell that is capable of silencing the pest gene in the plant cell.

According to an aspect of some embodiments of the present invention there is provided a method of producing a long dsRNA molecule in a plant cell that is capable of silencing a pest gene, the method comprising: (a) selecting a nucleic acid sequence of a plant gene exhibiting a predetermined sequence homology to a nucleic acid sequence of the pest gene; (b) modifying a plant endogenous nucleic acid sequence encoding an RNA molecule so as to impart silencing specificity towards the plant gene, such that small RNA molecules capable of recruiting RNA-dependent RNA Polymerase (RdRp) processed from the RNA molecule form base complementation with a transcript of the plant gene to produce the long dsRNA molecule capable of silencing the pest gene, thereby producing the long dsRNA molecule in the plant cell that is capable of silencing the pest gene.

According to an aspect of some embodiments of the present invention there is provided a method of generating a pest tolerant or resistant plant, the method comprising producing a long dsRNA molecule in a plant cell capable of silencing a pest gene according to some embodiments of the invention.

According to an aspect of some embodiments of the present invention there is provided a plant generated by the method of some embodiments of the invention.

According to an aspect of some embodiments of the present invention there is provided a cell of the plant of some embodiments of the invention.

According to an aspect of some embodiments of the present invention there is provided a seed of the plant of some embodiments of the invention.

According to an aspect of some embodiments of the present invention there is provided a method of producing a pest tolerant or resistant plant, the method comprising: (a) breeding the plant of some embodiments of the invention; and (b) selecting for progeny plants that express the long dsRNA molecule capable of suppressing the pest gene, and which do not comprise the DNA editing agent, thereby producing the pest tolerant or resistant plant.

According to an aspect of some embodiments of the present invention there is provided a method producing a plant or plant cell of some embodiments of the invention comprising growing the plant or plant cell under conditions which allow propagation.

According to some embodiments of the invention, the silencing molecule capable of recruiting the RdRp comprises 21-24 nucleotides.

According to some embodiments of the invention, the silencing molecule capable of recruiting the RdRp comprises 21 nucleotides.

According to some embodiments of the invention, the silencing molecule capable of recruiting the RdRp comprises 22 nucleotides.

According to some embodiments of the invention, the silencing molecule capable of recruiting the RdRp comprises 23 nucleotides.

According to some embodiments of the invention, the silencing molecule capable of recruiting the RdRp comprises 24 nucleotides.

According to some embodiments of the invention, the silencing molecule capable of recruiting the RdRp consists of 21 nucleotides.

According to some embodiments of the invention, the silencing molecule capable of recruiting the RdRp consists of 22 nucleotides.

According to some embodiments of the invention, the silencing molecule capable of recruiting the RdRp consists of 2.3 nucleotides.

According to some embodiments of the invention, the silencing molecule capable of recruiting the RdRp consists of 24 nucleotides.

According to some embodiments of the invention, the silencing molecule capable of recruiting the RdRp is selected from the group consisting of: trans-acting siRNA (tasiRNA), phased small interfering RNA (phasiRNA), microRNA (miRNA), small interfering RNA (siRNA), short hairpin RNA (shRNA), Piwi-interacting RNA (piRNA), transfer RNA (tRNA), small nuclear RNA (snRNA), ribosomal RNA (rRNA), small nucleolar RNA (snoRNA), extracellular RNA (exRNA), repeat-derived RNA, autonomous and non-autonomous transposable RNA.

According to some embodiments of the invention, the miRNA comprises a 22 nucleotides mature small RNA.

According to some embodiments of the invention, the miRNA is selected from the group consisting of: miR-156a, miR-156c, miR-162a, miR-162b, miR-167d, miR-169b, miR-173, miR-393a, miR-393b, miR-402, miR-447a, miR-447b, miR-447c, miR-472, miR-771, miR-777, miR-828, miR-830, miR-831, miR-831, miR-833a, miR-833a, miR-840, miR-845b, miR-848, miR-850, miR-853, miR-855, miR-856, miR-864, miR-2933a, miR-2933b, miR-2936, miR-4221, miR-5024, miR-5629, miR-5648, miR-5996, miR-8166, miR-8167a, miR-8167b, miR-8167c, miR-8167d, miR-8167e, miR-8167f, miR-8177 and miR-8182.

According to some embodiments of the invention, the plant gene is a non-protein coding gene.

According to some embodiments of the invention, the plant gene is a coding gene.

According to some embodiments of the invention, the plant gene does not encode for a molecule having an intrinsic silencing activity.

According to some embodiments of the invention, the method further comprises introducing into the plant cell a DNA editing agent conferring a silencing specificity of the plant gene towards the pest gene.

According to some embodiments of the invention, modifying of step (b) comprises introducing into the plant cell a DNA editing agent conferring the silencing specificity of the plant gene towards the pest gene.

According to some embodiments of the invention, the plant gene encodes for a molecule having an intrinsic silencing activity towards a native plant gene.

According to some embodiments of the invention, the method further comprises introducing into the plant cell a DNA editing agent which redirects a silencing specificity of the plant gene towards the pest gene, the pest gene and the native plant gene being distinct.

According to some embodiments of the invention, the method further comprises introducing into the plant cell a DNA editing agent which redirects a silencing specificity of the plant gene towards the pest gene, the pest gene and a native plant gene being distinct.

According to some embodiments of the invention, modifying of step (b) comprises introducing into the plant cell a DNA editing agent which redirects a silencing specificity of the plant gene towards the pest gene, the pest gene and a native plant gene being distinct.

According to some embodiments of the invention, the plant gene having the intrinsic silencing activity is selected from the group consisting of trans-acting siRNA (tasiRNA), phased small interfering RNA (phasiRNA), microRNA (miRNA), small interfering RNA (siRNA), short hairpin RNA (shRNA), Piwi-interacting RNA (piRNA), transfer RNA (tRNA), small nuclear RNA (snRNA), ribosomal RNA (rRNA), small nucleolar RNA (snoRNA), extracellular RNA (exRNA), autonomous and non-autonomous transposable RNA.

According to some embodiments of the invention, the plant gene having the intrinsic silencing activity encodes for a phased secondary siRNA-producing molecule.

According to some embodiments of the invention, the plant gene having the intrinsic silencing activity is a trans-acting-snRNA-producing (TAS) molecule.

According to some embodiments of the invention, the silencing specificity of the plant gene is determined by measuring a transcript level of the pest gene.

According to some embodiments of the invention, the silencing specificity of the plant gene is determined phenotypically.

According to some embodiments of the invention, determined phenotypically is effected by determination of pest resistance of the plant.

According to some embodiments of the invention, the silencing specificity of the plant gene is determined genotypically.

According to some embodiments of the invention, the plant phenotype is determined prior to a plant genotype.

According to some embodiments of the invention, the plant genotype is determined prior to a plant phenotype.

According to some embodiments of the invention, the silencing specificity of the plant gene is determined by measuring a transcript level of the pest gene.

According to some embodiments of the invention, the determined phenotypically is effected by determination of pest resistance of the plant.

According to some embodiments of the invention, the predetermined sequence homology comprises 75-100% identity.

According to some embodiments of the invention, the small RNA molecules capable of recruiting the RdRp comprise 21-24 nucleotides.

According to some embodiments of the invention, the small RNA molecules capable of recruiting the RdRp comprise 21 nucleotides.

According to some embodiments of the invention, the small RNA molecules capable of recruiting the RdRp comprise 22 nucleotides.

According to some embodiments of the invention, the small RNA molecules capable of recruiting the RdRp comprise 23 nucleotides.

According to some embodiments of the invention, the small RNA molecules capable of recruiting the RdRp comprise 24 nucleotides.

According to some embodiments of the invention, the small RNA molecules capable of recruiting the RdRp consist of 21 nucleotides.

According to some embodiments of the invention, the small RNA molecules capable of recruiting the RdRp consist of 22 nucleotides.

According to some embodiments of the invention, the small RNA molecules capable of recruiting the RdRp consist of 23 nucleotides.

According to some embodiments of the invention, the small RNA molecules capable of recruiting the RdRp consist of 24 nucleotides.

According to some embodiments of the invention, the small RNA molecules capable of recruiting the RdRp are selected from the group consisting of microRNA (miRNA), small interfering RNA (siRNA), short hairpin RNA (shRNA), Piwi-interacting RNA (piRNA), transacting siRNA (tasiRNA), phased small interfering RNA (phasiRNA), transfer RNA (tRNA), small nuclear RNA (snRNA), ribosomal RNA (rRNA), small nucleolar RNA (snoRNA), extracellular RNA (exRNA), repeat-derived RNA, autonomous and non-autonomous transposable RNA.

According to some embodiments of the invention, the RNA molecule does not have an intrinsic silencing activity.

According to some embodiments of the invention, the method further comprises introducing into the plant cell a DNA editing agent conferring a silencing specificity of the RNA molecule towards the plant gene.

According to some embodiments of the invention, the RNA molecule has an intrinsic silencing activity towards a native plant gene.

According to some embodiments of the invention, the method further comprises introducing into the plant cell a DNA editing agent which redirects a silencing specificity of the RNA molecule towards the plant gene, the plant gene and the native plant gene being distinct.

According to some embodiments of the invention, modifying of step (b) comprises introducing into the plant cell a DNA editing agent which redirects a silencing specificity of the RNA molecule towards the plant gene, the plant gene and a native plant gene being distinct.

According to some embodiments of the invention, the plant gene exhibiting the predetermined sequence homology to the nucleic acid sequence of the pest gene does not encode a silencing molecule.

According to some embodiments of the invention, the silencing specificity of the RNA molecule is determined by measuring a transcript level of the plant gene or the pest gene.

According to some embodiments of the invention, the silencing specificity of the RNA molecule is determined phenotypically.

According to some embodiments of the invention, the determined phenotypically is effected by determination of pest resistance of the plant.

According to some embodiments of the invention, the silencing specificity of the RNA molecule is determined genotypically.

According to some embodiments of the invention, the plant phenotype is determined prior to a plant genotype.

According to some embodiments of the invention, the plant genotype is determined prior to a plant phenotype.

According to some embodiments of the invention, the DNA editing agent comprises at least one sgRNA.

According to some embodiments of the invention, the DNA editing agent comprises at least one sgRNA operatively linked to a plant expressible promoter.

According to some embodiments of the invention, the DNA editing agent does not comprise an endonuclease.

According to some embodiments of the invention, the DNA editing agent comprises an endonuclease.

According to some embodiments of the invention, the DNA editing agent is of a DNA editing system selected from the group consisting of a meganuclease, a zinc finger nucleases (ZFN), a transcription-activator like effector nuclease (TALEN), CRISPR-endonuclease, dCRISPR-endonuclease and a homing endonuclease.

According to some embodiments of the invention, the endonuclease comprises Cas9.

According to some embodiments of the invention, the DNA editing agent is applied to the cell as DNA, RNA or RNP.

According to some embodiments of the invention, the DNA editing agent is linked to a reporter for monitoring expression in a plant cell.

According to some embodiments of the invention, the reporter is a fluorescent protein.

According to some embodiments of the invention, the plant cell is a protoplast.

According to some embodiments of the invention, the dsRNA molecule is processable by cellular RNAi processing machinery.

According to some embodiments of the invention, the dsRNA molecule is processed into secondary small RNAs.

According to some embodiments of the invention, the dsRNA and/or the secondary small RNAs comprise a silencing specificity towards a pest gene.

According to some embodiments of the invention, the pest is an invertebrate.

According to some embodiments of the invention, the pest is selected from the group consisting of a virus, an ant, a termite, a bee, a wasp, a caterpillar, a cricket, a locust, a beetle, a snail, a slug, a nematode, a bug, a fly, a fruitfly, a whitefly, a mosquito, a grasshopper, a planthopper, an earwig, an aphid, a scale, a thrip, a spider, a mite, a psyllid, a tick, a moth, a worm, a scorpion and a fungus.

According to some embodiments of the invention, the plant is selected from the group consisting of a crop, a flower, a weed, and a tree.

According to some embodiments of the invention, the plant is non-transgenic.

According to some embodiments of the invention, the plant is a transgenic plant.

According to some embodiments of the invention, the plant is non-genetically modified (non-GMO).

According to some embodiments of the invention, the plant is genetically modified (GMO).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a is a photograph illustrating the first proposed model (referred to as Model 1) for target gene amplification by Gene Editing induced Gene Silencing (GEiGS). According to this model (see the corresponding numbers in the figures):

1. The pest gene “X” is the target gene (when silenced, the pest is controlled)

2. A host-related gene-X is identified by homology search (plant gene “X”)

3. GEiGS is performed to redirect the silencing specificity of an amplifier small RNA (e.g. 22nt miRNAs) against the plant gene “X”.

4. The amplifier small GEiGS RNA forms a RISC complex that is associated with RdRp (the amplifying enzyme)

5. The RdRp synthesizes a complementary antisense RNA strand to the transcript of plant gene “X”, forming dsRNA.

6. The plant gene “X” dsRNA is processed into secondary sRNAs by dicer(s) or dicer-like proteins.

7. The plant gene “X” dsRNA is taken up by pests. Within the pest, the plant dsRNA-X is processed into small RNAs that down-regulate via RNAi the corresponding homologous pest gene “X”.

8. Possibly, secondary sRNAs are taken up by pests, and silence the target gene “X”

FIG. 2 is a photograph illustrating the second proposed model (referred to as Model 2) for target gene amplification by GEiGS. According to this model (see the corresponding numbers in the figures):

1. The pest gene “X” is the target gene (when silenced, the pest is controlled)

2. GEiGS is performed to redirect the silencing specificity of naturally occurring amplified RNAi precursor against the pest gene “X” (e.g. TAS; amplified and processed into tasiRNAs)

3. A wild type amplifier sRNA forms a RISC complex that is associated with RdRp (the amplifying enzyme)

4. The RdRp synthesizes a complementary antisense RNA strand to the transcript of the amplified GEiGS precursor, forming dsRNA

5. The amplified GEiGS dsRNA is processed into secondary sRNAs by dicer(s)

6. The GEiGS dsRNA is taken up by pests. Within the pest, the plant GEiGS-dsRNA is processed into small RNAs that down-regulate via RNAi the corresponding homologous pest gene “X”

7. Possibly, secondary sRNAs derived from the GEiGS-dsRNA (e.g. tasiRNAs in the case of TAS precursor) are taken up as well by the pest, and silence the target gene “X”

FIG. 3A illustrates identification of endogenous genes in the plant with regions homologous to the pest sequence (per model 1). Specifically, blast alignment of AF502391.1 (H. glycines, SEQ ID NO: 1) pest against NM_001037071.1 (A. thaliana, SEQ ID NO: 2) plant gene.

FIG. 3B illustrates miRNA based GEiGS oligo designed carrying siRNA sequences targeting a region downstream of the region of homology in the plant (described in FIG. 3A). Top: GEiGS oligo, SEQ ID NO: 3 (siRNA in red). Bottom: plant target gene carrying homology to pest (SEQ ID NO: 4). Homologous pest sequence in green (SEQ ID NO: 1). The sequence predicted to be targeted by the GEiGS-siRNA is in red.

FIG. 4A illustrates identification of endogenous genes in the plant with regions homologous to the pest sequence (per model 1). Specifically, blast alignment of AF500024.1 (H. glycines, SEQ ID NO: 5) pest against NM_116351.7 (A. thaliana, SEQ ID NO: 6) plant gene.

FIG. 4B illustrates miRNA based GEiGS oligo designed carrying siRNA sequences targeting a region downstream of the region of homology in the plant (described in FIG. 4A). Top: GEiGS oligo, SEQ ID NO: 7 (siRNA in red). Bottom: target gene carrying homology to pest (SEQ ID NO: 8). Homologous pest sequence in green (SEQ ID NO: 5). The sequence predicted to be targeted by the GEiGS-siRNA is in red.

FIG. 5A illustrates identification of endogenous genes in the plant with regions homologous to the pest sequence (per model 1). Specifically, blast alignment of AF469060.1 (H. glycines, SEQ ID NO: 9) pest against NM_001203752.2 (A. thaliana, SEQ ID NO: 10) plant gene.

FIG. 5B illustrates miRNA based GEiGS oligo designed carrying siRNA sequences targeting a region downstream of the region of homology in the plant (described in FIG. 5A). Top: GEiGS oligo, SEQ II) NO: 11 (siRNA in red). Bottom: target gene carrying homology to pest (SEQ ID NO: 12). Homologous pest sequence in green (SEQ ID NO: 9). The sequence predicted to be targeted by the GEiGS-siRNA is in red.

FIG. 6 is an embodiment flow chart of computational pipeline to generate GEiGS templates. The computational GeiGS pipeline applies biological metadata and enables an automatic generation of GeiGS DNA donor templates that are used to minimally edit endogenous non-coding RNA genes (e.g. miRNA genes), leading to a new gain of function, i.e. redirection of their silencing capacity to target gene expression of interest.

FIG. 7 is an embodiment flow chart of Genome Editing induced Gene Silencing (GEiGS) replacement of endogenous miRNA with siRNA targeting the PDS gene, hence inducing gene silencing of the endogenous PDS gene. To introduce the modification, a 2-component system is being used. First, a CRISPR/CAS9 system, in a GIP containing vector, generates a cleavage in the chosen loci, through designed specific guide RNAs to promote homologous DNA repair (HDR) in the site. Second, A DONOR sequence, with the desired modification of the miRNA sequence, to target the newly assigned genes, is introduced as a template for the HDR. This system is being used in protoplast transformation, enriched by FACS due to the GIP signal in the CRISPR/CAS9 vector, recovered, and regenerated to plants.

FIGS. 8A-C are photographs illustrating that silencing of the PDS gene causes photobleaching. Silencing of the PDS gene in Nicotiana (FIGS. 8A-B) and Arahidopsis (FIG. 8C) plants causes photobleaching in N. benthamiana (FIG. 8B) and Arahidopsis (FIG. 8C, right side). Photographs were taken 3½ weeks after PDS silencing.

FIG. 9A depicts a schematic representation of an example of HDR-mediated genomic swaps in Col-0 cells and primers used for PCR and genotyping of such swaps. The CRISPR/Cas9 and sgRNA targeted the swap region, generating a dsDNA break. The DONOR templates carried homologous arms for insertion by homology directed repair (HDR) into that genomic locus (AtTAS1b or AtTAS3a), introducing the desired swaps. Swap region: sequence that was modified to target nematode genes. Short arrows represent the swap-specific or wt-specific forward primer and unspecific reverse primer, common for all reactions, used for PCR to demonstrate genomic swaps. The reverse primer was designed to anneal further downstream the recombination site, to avoid amplification of the DONOR template. Swap-specific forward primers were designed in such a way that they only allowed amplification if a swap took place. An additional forward primer was designed for control PCR amplification on wild-type (WT) sequence only. The dotted line represents the PCR product. The oval indicates the reverse primer used for Sanger sequencing reactions.

FIGS. 9B-C depict micrographs of electrophoresis of PCR products generated with WT primers. The unspecific reverse primer and a WT specific primer were used for PCR on DNA extracted from all treatments described in Example 3. PCR products were run on 1.6% agarose gels. Small arrows and numbers indicate bands and sizes for the expected PCR products. (FIG. 9B) represents PCR reactions for AtTAS1b loci and (FIG. 9C) represents reactions for AtTAS3a loci. Y25: Y25, beta subunit of COPI complex; Splicing: Splicing factor; Ribo3a: Ribosomal protein 3a; Spliceo: Spliceosomal SR protein; WT: wild-type. H₂O: no template, water negative PCR controls. MW: 1 kb plus molecular weight ladder (NEB).

FIGS. 9D-E depict micrographs of electrophoresis of PCR products generated with swap specific primers. The unspecific reverse primer and a swap specific forward primer were used for PCR on DNA extracted from all swap treatments in Example 3. As a control for the specificity of the reaction WT DNA was also used as template. PCR products were run on 1.6% agarose gels. Small arrows and numbers indicate bands and sizes for the expected PCR products. (FIG. 9D) represents PCR reactions for swaps at AtTAS1b (Tas1b) loci and (FIG. 9E) represents reactions for swaps at AtTAS3a (Tas3a) loci. Y25: Y25, beta subunit of COPT complex; Splicing: Splicing factor; Ribo3a: Ribosomal protein 3a; Spliceo: Spliceosomal SR protein; WT: wild-type. H₂O: no template, water negative PCR controls. MW: 1 kb plus molecular weight ladder (NEB).

FIGS. 9F-G depict a scheme of a Sanger sequencing reaction of PCR products. The unspecific reverse primer from FIG. 9A was used for Sanger sequencing of each PCR product. Arrows represents the specific forward primers used for PCR. amplification. Additional nucleotide changes introduced following HDR event (not originating from the primer used in the reaction) are displayed highlighted and greyed out. Chromatograms show the sequences for the PCR products, which were aligned against the predicted sequences (upper line). (FIG. 9F) represents sequencing reactions for swaps at AtTAS1b (Tas1b) loci and (FIG. 9G) represents reactions for swaps at AtTAS3a (Tas3a) loci. Y25: Y25, beta subunit of COPT complex; Splicing: Splicing factor; Ribo3a: Ribosomal protein 3a; Spliceo: Spliceosomal SR protein; WT: wild-type.

FIGS. 10A-B depict schematic representations of a Sense (FIG. 10A) and Anti-sense (FIG. 10B) strand of dsRNA generated through HDR-mediated genomic swaps in Col-0 cells. Swap region: sequence that was modified to target nematode genes. Short arrows represent the unspecific primers used for reverse transcription PCR (RT-PCR) and for cDNA generation. Additional short arrows represent the swap-specific primer and unspecific primer, common for all reactions, used for PCR (PCR) on cDNA to prove swap expression. PCR reactions were designed in such a way that the length for all PCR products was lower than 200 nucleotides. Specific primers were designed in such a way that they only allowed amplification if a swap took place. The dotted lines represent the expected PCR products. The oval indicates the primers used for Sanger sequencing reactions. Direction is indicated for transcripts from 5′ to 3′.

FIGS. 10C-D depict micrographs of electrophoresis of PCR products to examine expression of AtTAS1b Sense and Anti-sense RNA strands to detect dsRNA containing swaps. RT-PCR reactions were carried out to generate cDNA and subsequent PCR reactions were carried out using the primers described in FIGS. 10A-B. PCR products were run on 1.6% agarose gels. Small arrows and numbers indicate bands and sizes for the expected PCR products. (FIG. 10C) represents PCR reactions for AtTAS1b Sense RNA transcript and (FIG. 10D) represents PCR reactions for AtTAS1b Anti-sense RNA transcripts. Y25: Y25, beta subunit of COPI complex; WT: wild-type; H₂O: no template, water negative PCR controls; MW: 1 kb plus molecular weight ladder (NEB), +RT: PCR reactions using cDNA amplified by reverse transcriptase as template. −RT: reverse transcription controls—No reverse transcriptase was used and no cDNA was generated.

FIGS. 10E-F depict micrographs of electrophoresis of PCR products to examine expression of AtTAS3a Sense and Anti-sense RNA strands to detect dsRNA containing swaps. RT-PCR reactions were carried out to generate cDNA and subsequent PCR reactions were carried out using the primers described in FIGS. 10A-B. PCR products were run on 1.6% agarose gels. Small arrows and numbers indicate bands and sizes for the expected PCR products. (FIG. 10E) represents PCR reactions for AtTAS3a Sense RNA transcript and (FIG. 10F) represents PCR reactions for AtTAS3a Anti-sense RNA transcripts. Ribo3a: Ribosomal protein 3a; WT: wild-type. H₂O: no template, water negative PCR controls. MW: 1 kb plus molecular weight ladder (NEB). +RT: PCR reactions using cDNA, amplified by reverse transcriptase, as template. −RT: reverse transcription controls—No reverse transcriptase was used and no cDNA was generated.

FIG. 10G depicts a scheme of a Sanger sequencing reaction of PCR products that amplified the Sense strand of RNA with introduced swaps. The unspecific forward primer from FIG. 10A was used for Sanger sequencing of each PCR product. Arrows represent the specific reverse primers used for PCR amplification. Additional nucleotide changes introduced by DONOR template are displayed highlighted and greyed out. Chromatograms show the sequences for the PCR products, which were aligned against the predicted sequences. Top panel represents sequencing reactions for expression proof for swap in the AtTAS1b (Tas1b) loci and bottom panel is represents reactions for expression proof for swap in the AtTAS3a (Tas3a;) loci. Y25: Y25, beta subunit of COPI complex; Ribo3a: Ribosomal protein 3a; WT: wild-type.

FIG. 10H depicts a scheme of a Sanger sequencing reaction of PCR products that amplified the Anti-Sense strand of RNA with introduced swaps. The unspecific reverse primer from FIG. 10B was used for Sanger sequencing of each PCR product. Arrows represent the specific forward primers used for PCR amplification. Additional nucleotide changes introduced by DONOR template are displayed highlighted and greyed out. Chromatograms show the sequences for the PCR products, which were aligned against the predicted sequences. Top row represents sequencing reactions for expression proof for swap at AtTAS1b (Tas1b) loci and bottom row represents reactions for expression proof for swap at AtTAS3a (Tas3a) loci. Y25: Y25, beta subunit of COPI complex; Ribo3a: Ribosomal protein 3a; WT: wild-type.

FIG. 10I depicts a scheme of a Sanger sequencing reaction of PCR products that amplified the Sense and Anti-Sense strands of wild-type RNA transcribed from Tas1b and Tas3a. For sense transcripts the unspecific forward primer from FIG. 10A was used for Sanger sequencing of each PCR product. For antisense transcripts the unspecific reverse primer from FIG. 10B was used for Sanger sequencing of each PCR product. Arrows represent the forward primers used for PCR amplification. Chromatograms show the sequences for the PCR products, which were aligned against the annotated WT sequences.

FIG. 11A provides in the lower panel a bar-graph depicting levels of TuMV infection in leaves of N. Benthamiana following inoculation with various treatments, as represented by measuring relative expression through quantification of TuMV transcript levels and GFP visualisation. Control and treatments were infiltrated side-by-side on the same leaf. Left to right—(1) Leaf was infiltrated with agrobacterium containing TuMV vector (n=3; left side of the leaf) or agrobacterium without any vector (n=3; right side of the leaf). (2) Leaf was infiltrated with agrobacterium containing a vector overexpressing miR173 (n=3; left side) or with agrobacterium containing no vector (n=3; right side). (3) Leaf was infiltrated with a vector overexpressing the GEiGS-dummy (n=3; left side) or GEiGS-TuMV (n=3; right side). (4) Leaf was infiltrated with agrobacterium containing a vector overexpressing the GEiGS-dummy (n=3; left side) or agrobacterium containing a vector endocing the GEiGS-TuMV (n=2; tight side), both co-infiltrated with agrobacterium containing a vector overexpressing miR173. The micrographs in the upper panel are representative pictures of the analysed samples. TuMV was monitored through GFP signal, visualised under UV light. Bars indicate average values; Error bars represent standard error; *-p-value<0.05; **-p-value<0.01 according to One-way ANOVA and post-hoc Tukey HSD test.

FIG. 11B provides photographs depicting whole N. benthamiana leaves which have been co-infiltrated with agrobacterium containing vectors overexpressing GEiGS-dummy and miR173 (centre) or overexpressing GEiGS-TuMV and miR173 (right). Control leaf was infiltrated with agrobacterium containing no vector (left). TuMV was monitored through GFP signal, visualised under UV light.

FIG. 12A is a bar graph providing relative expression of Ribosomal protein 3a in nematodes fed with total RNA extracted from N. benthamiana leaves which were co-infiltrated with vectors overexpressing miR390 and TAS3a which was modified to target Ribosomal protein 3a. Nematodes fed with RNA from explants overexpressing the TAS3a wt backbone and the miR390 amplifier were used as control. Analysis was carried out on nematodes fed during 3 days with the RNA extract, by qRT-PCR, using actin as endogenous normaliser gene, (Error bars represent standard error; ***-p-value<0.001).

FIG. 12B is a bar graph providing relative expression of Spliceosomal SR protein in nematodes fed with total RNA extracted from N. benthamiana leaves which were co-infiltrated with vectors overexpressing miR390 and TAS3a which was modified to target Spliceosomal SR protein. Nematodes fed with RNA from explants overexpressing the TAS3a wt backbone and the miR390 amplifier were used as control. Analysis was carried out on nematodes fed during 3 days with the RNA extract, by qRT-PCR, using actin as endogenous normaliser gene. (Error bars represent standard error; **-p-value<0.01).

FIGS. 13A-D depict RNA-seq analysis (FIGS. 13A and 13C) and small RNA-seq analysis (FIGS. 13B and 13D) of N. benthamiana leaves infiltrated with vectors expressing GEiGS designs against ribosomal protein 3a (FIGS. 13A and 13B) and Spliceosomal SR protein (FIGS. 13C and 13D), and miR390, aligned to the GEiGS design, 48 to 72 hours post infiltration. Light grey rectangles in each plot indicate the region of miR390 binding on the transcript. The black squares in each plot indicate the homology region to the target genes that give rise to the secondary siRNA that target the genes in nematodes. Top chromatograms in each plot indicate the sense strand while the bottom ones indicate the anti-sense.

### DESCRIPTION OF SPECIFIC EMBODIMENTS OF THE INVENTION

The present invention, in some embodiments thereof, relates to generation and amplification of dsRNA molecules in a host cell for silencing pest target genes.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways and in different organisms. Also, it is to be understood that the phraseology and terminology employed herein is for the purpose of description and should not be regarded as limiting.

Previous work on genome editing of RNA molecules in various organisms (e.g. murine, human, plants), focused on disruption of miRNA activity or target binding sites using transgenesis. Genome editing in plants has concentrated on the use of nucleases such as CRISPR-Cas9 technology, ZFNs and TALENs, for knockdown of genes or insertions in model plants. Furthermore, gene silencing in plants using artificial miRNA transgenes to silence endogenous and exogenous target genes has been described [Molnar A et al. Plant J. (2009) 58(1)165-74. doi: 10.1111/j.1365-313X.2008.03767.x. Epub 2009 Jan. 19; Borges and Martienssen, Nature Reviews Molecular Cell Biology| AOP, published online 4 Nov. 2015; doi:10.1038/nrm4085]. The artificial miRNA transgenes are introduced into plant cells within an artificial expression cassette (including a promoter, terminator, selection marker, etc.) and downregulate target expression.

Recent advances in genome editing techniques have made it possible to alter DNA sequences in living cells by editing one or more a few nucleotides in cells of human patients such as by genome editing (NHEJ and HR) following induction of site-specific double-strand breaks (DSBs) at desired locations in the genome. While NHEJ is mainly, if not exclusively, used for knockout purposes, HR is used for introducing precision editing of specific sites such as point mutations or correcting deleterious mutations that are naturally occurring or hereditarily transmitted. p Mature small RNAs (i.e. dicer products and non-dicer products) and dsRNA (i.e. dicer substrates, e.g. small RNA precursors) can mediate efficient cellular gene knockdown. The biogenesis of miRNAs involves the presence of dsRNA structures (e,g. hairpin precursors). However, the hairpin RNA may not be efficiently taken up by pests because: (i) quantity is low due to its instability (e.g. processed by dicer) and; (ii) the lack of RNA-RNA amplification stage by RNA-dependent RNA-polymerases (RdRp). Accordingly, pests are more susceptible to ingested small RNA precursors (e.g. dsRNA).

While reducing the present invention to practice, the present inventors have devised a gene editing technology directed to generation of long dsRNA molecules in plant cells and tissues for targeting of pest genes. Such dsRNA molecules can be mobile and transferred among cells and tissues; hence can occur outside cells once produced in cells. Furthermore, such dsRNA molecules can be transferred between organisms through ingestion of material derived from the dsRNA-expressing host (e.g. plant leaves and stems). Specifically, the present inventors have developed a GEiGS system that involves one of two models.

The below-described models are based in part on the Gene Editing induced Gene Silencing (GEiGS) technology as described in WO 2019/058255, which is hereby incorporated by reference in its entirety. As used herein, the phrase “GEiGS is performed” relates to use of the GEiGS technology in order to redirect silencing specificity of a silencing RNA, which essentially includes modifying a nucleic acid sequence encoding a silencing RNA, such that the encoded silencing RNA targets a target of choice. According to some embodiments, GEiGS is performed by inducing a double-strand break in the nucleic acid sequence encoding the silencing RNA in a cell (e.g. by expressing or introducing an endonuclease into the cell, such as, but not limited to Cas9), and providing a nucleic acid template which includes the desired nucleotide changes in the nucleic acid sequence encoding the silencing RNA. According to such embodiments, the nucleotide changes are then introduced into the nucleic acid sequence encoding the silencing RNA via Homology Dependent Recombination (HDR) as the relevant part of the nucleic acid template is introduced. According to some embodiments, the nucleic acid template introduces nucleotides changes in the nucleic acid sequence encoding the silencing RNA, such that the silencing RNA targets a target sequence of choice. Examples of using GEiGS to change nucleotides in a nucleic acid sequence encoding a miRNA or a tasiRNA are exemplified herein below in Examples 1B and 3.

In the first model, a plant gene is identified which is homologous to a pest target gene. GEiGS is performed to redirect the silencing specificity of a small RNA molecule against the plant gene (being homologous to the pest target gene). This small RNA molecule (also referred to as an amplifier or primer small RNA) forms a complex with RdRp, and RdRp synthesizes a complementary anti-sense RNA strand to the transcript of the plant gene, forming a dsRNA. The dsRNA is then further processed into secondary small RNAs (sRNAs). Importantly, the primary small RNAs, dsRNA, as well as the secondary small RNA molecules (i.e. the product of RNAi processing of the newly generated dsRNAs, e.g. by Dicer-like) are taken up by the pest and can mediate pest gene silencing. Essentially, by re-directing the targeting specificity of an amplifier small RNA molecule using GEiGS, the first model enables formation of a novel long-dsRNA from a sequence which did not previously form a long dsRNA, thus resulting in a phased-RNA producing locus. As this locus carries a natural similarity to a pest gene, a resulting long dsRNA harbors the capacity to silence the corresponding gene within the pest.

In the second model, GEiGS is performed on a plant gene, which is naturally converted into double stranded RNA form (a naturally amplified locus which produces a long dsRNA and phased-RNAs, e.g. a naturally occurring TAS), to redirect a silencing specificity towards a pest target gene. Initially, a native silencing RNA molecule (also referred to herein as an amplifier or primer small RNA; e.g. 22 nt miRNA such as miR-173) is selected which has the plant gene as a target and which is capable of forming a complex with RdRp. RdRp synthesizes a complementary anti-sense RNA strand to the transcript of the plant gene, forming a long dsRNA. The long dsRNA is then further processed into secondary sRNAs (i.e. the product of RNAi processing of the newly generated dsRNAs, e.g. by Dicer-like). According to this model, the long dsRNA as well as the secondary small RNA molecules are taken up by the pest and can mediate pest gene silencing.

Thus, the present invention provides formation of amplifiable dsRNA molecules in plant cells and tissues with projected larger quantity as well as larger small RNA population and hence with much higher silencing efficacy. Furthermore, the multiple secondary small RNAs generated from the dsRNA molecules increases the chances of efficient target knockdown. The dsRNA molecules produced by the present methods are taken up efficiently by pests enabling an efficient gene silencing and safe control of pest genes without harming the plants. Furthermore, the gene editing technology described herein does not implement the classical molecular genetic and transgenic tools comprising expression cassettes that have a promoter, terminator, selection marker.

Thus, according to one aspect of the present invention there is provided a method of producing a long dsRNA molecule in a plant cell that is capable of silencing a pest gene, the method comprising:

(a) selecting a nucleic acid sequence of a plant gene exhibiting a predetermined sequence homology to a nucleic acid sequence of the pest gene;

(b) modifying a plant endogenous nucleic acid sequence encoding an RNA molecule so as to impart silencing specificity towards the plant gene, such that small RNA molecules capable of recruiting RNA.-dependent RNA Polymerase (RdRp) processed from the RNA molecule form base complementation with a transcript of the plant gene to produce the long dsRNA molecule capable of silencing the pest gene,

thereby producing the long dsRNA molecule in the plant cell that is capable of silencing the pest gene.

The term “long dsRNA molecule” as used herein refers to double-stranded sequences of polyribonucleic acids having a first strand (sense strand) and a second strand that is a reverse is complement of the first strand (anti-sense strand), the polyribonucleic acids held together by base pairing (e.g., two sequences that are the reverse complement of each other in the region of base pairing), wherein the double stranded polyribonucleic acid can be a substrate for an enzyme from the Dicer family, typically wherein the long dsRNA molecule is at least 26 bp or longer. The two strands can be of identical length or of different lengths provided there is enough sequence homology between the two strands that a stable double stranded structure is formed with at least 80 %, 85%, 90%, 95%, 97%, 99% or 100% complementarity over the entire length.

By use of the term “complementation”, “complementarity” or “complementary” is meant that the RNA molecules (or at least a portion of it that is present in the processed small RNA form, or at least one strand of a double-stranded polynucleotide or portion thereof, or a portion of a single strand polynucleotide) hybridizes under physiological conditions to the target RNA (e.g, transcript of the plant gene), or a fragment thereof, to effect regulation or function of RdRp mediated synthesis of the target gene. For example, in some embodiments, a RNA molecule (e.g. small RNA molecule) has 100% sequence identity or at least about 30, 40, 45, 50, 55, 60, 65, 70, 75, 80, 83, 84, 85, 86, 87, 88, 89, 90, 91, 92, 93, 94, 95, 96, 97, 98, or 99% sequence identity when compared to a sequence of 10, 11, 12, 13. 14, 15, 16, 17, 18, 19, 20, 21. 22, 23, 24, 25. 26, 27, 28, 29. 30, 31, 32 33, 33, 34, 35, 36, 37, 38, 39, 40, 41, 42, 43, 44, 45, 46, 47, 48 49, 50, 51, 52, 53, 54, 55, 56, 57, 58, 59, 60, 70, 80, 90, 100, 150, 200, 300, 400, 500 or more contiguous nucleotides in the target RNA (or family members of a given target gene).

As used herein, a RNA molecule, or it's processed small RNA forms (discussed in further detail hereinbelow), are said to exhibit “complete complementarity” when every nucleotide of one of the sequences read 5′ to 3′ is complementary to every nucleotide of the other sequence when read 3′ to 5′. A nucleotide sequence that is completely complementary to a reference nucleotide sequence will exhibit a sequence identical to the reverse complement sequence of the reference nucleotide sequence.

Methods for determining sequence complementarity are well known in the art and include, but are not limited to, bioinformatics tools which are well known in the art (e.g. BLAST, multiple sequence alignment).

According to one embodiment, the long dsRNA molecule is longer than 20 bp.

According to one embodiment, the long dsRNA molecule is longer than 21 bp.

According to one embodiment, the long dsRNA molecule is longer than 22 bp.

According to one embodiment, the long dsRNA molecule is longer than 23 bp.

According to one embodiment, the long dsRNA molecule is longer than 24 bp.

According to one embodiment, the long dsRNA molecule comprises 20-100,000 bp.

According to one embodiment, the long dsRNA molecule comprises 20-10,000 bp.

According to one embodiment, the long dsRNA molecule comprises 20-1,000 bp.

According to one embodiment, the long dsRNA molecule comprises 20-500 bp.

According to one embodiment, the long dsRNA molecule comprises 20-50 bp.

According to one embodiment, the long dsRNA molecules comprise 200-5000 bp.

According to one embodiment, the long dsRNA molecules comprise 200-1000 bp.

According to one embodiment, the long dsRNA molecules comprise 200-500 bp.

According to one embodiment, the long dsRNA molecules comprise 2000-100,000 bp.

According to one embodiment, the long dsRNA molecules comprise 2000-10,000 bp.

According to one embodiment, the long dsRNA molecules comprise 2000-5000 bp.

According to one embodiment, the long dsRNA molecules comprise 10,000-100,000 bp.

According to one embodiment, the long dsRNA molecules comprise 1,000-10,000 bp.

According to one embodiment, the long dsRNA molecules comprise 100-10,000 bp.

According to one embodiment, the long dsRNA molecules comprise 100-1,000 bp.

According to one embodiment, the long dsRNA molecules comprise 10-1,000 bp.

According to one embodiment, the long dsRNA molecules comprise 10-100 bp.

According to one embodiment, the long dsRNA molecule comprises an overhang, i.e. a non-double stranded region of a dsRNA molecule (i.e., single stranded RNA).

According to one embodiment, the long dsRNA molecule does not comprise an overhang.

According to one embodiment, the long dsRNA molecule of the invention can be processed into small RNA molecules capable of engaging with RNA-induced silencing complex (RISC). Accordingly, the long dsRNA molecule of the invention may serve as a substrate for the intra-cellular RNAi processing machinery (i.e. may be a precursor RNA molecule) and may be processed by ribonucleases, including but not limited to, the DICER protein family (e.g. DCR1 and DCR2), DICER-LIKE protein family (e.g. DCL1, DCL2, DCL3, DCL4), ARGONAUTE protein family (e.g. AGO1, AGO2, AGO3, AGO4), tRNA cleavage enzymes (e.g. RNY1, ANGIOGENIN, RNase P, RNase P-like, SLFN3, ELAC1 and ELAC2), and Piwi-interacting RNA (piRNA) related proteins (e.g. AGO3, AUBERGINE, HIWI, HIWI2, HIWI3, PIWI, ALG1 and ALG2) into small RNA molecules, as discussed in detail hereinbelow.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that may be useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Cannabaceae, Cannabis indica, Cannabis, Cannabis saliva, Hemp, industrial Hemp, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Elealia vi/losa, Pagopyrum spp., Feijoa sellowlana, Fragaria spp., Fleminia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculents, Medicago saliva, Metasequoia glyptostroboides, Musa sapientum, banana, Nicotianum spp., Onobrychis spp., Ornithopus spp., Oryza spp., Peltophorum africanum, Pennisetum spp., Persia gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachytium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

According to a specific embodiment, the plant is a crop, a flower, a weed or a tree.

According to a specific embodiment, the plant is a woody plant species e.g., Actinidia chinensis (Actinidiaceae), Manihotesculenta (Euphorbiaceae), Firiodendron tulipifera (Magnoliaceae), Populus (Salicaceae), Santalum album (Santalaceae), Ulmus (Ulmaceae) and different species of the Rosaceae (Malus, Prunus, Pyrus) and the Rutaceae (Citrus, Microcitrus), Gymnospermae e.g., Picea glauca and Pinus taeda, forest trees (e.g., Betulaceae, Fagaceae, Gymnospermae and tropical tree species), fruit trees, shrubs or herbs, e.g., (banana, cocoa, coconut, coffee, date, grape and tea) and oil palm.

According to a specific embodiment, the plant is of a tropical crop e.g., coffee, macadamia, banana, pineapple, taro, papaya, mango, barley, beans, cassava, chickpea, cocoa (chocolate), cowpea, maize (corn), millet, rice, sorghum, sugarcane, sweet potato, tobacco, taro, tea, yam.

“Grain,” “seed,” or “bean,” refers to a flowering plant's unit of reproduction, capable of developing into another such plant. As used herein, the terms are used synonymously and interchangeably.

According to a specific embodiment, the plant is a plant cell e.g., plant cell in an embryonic cell suspension.

According to a specific embodiment, the plant cell is a protoplast.

The protoplasts are derived from any plant tissue e.g., fruit, flowers, roots, leaves, embryos, embryonic cell suspension, calli or seedling tissue.

According to a specific embodiment, the plant cell is an embryogenic cell.

According to a specific embodiment, the plant cell is a somatic embryogenic cell.

The term “plant gene” as used herein refers to any gene in the plant, e.g., endogenous, that can be modified as to impart silencing specificity towards a pest gene.

According to one embodiment, the plant gene is a non-coding gene (e.g. non-protein coding gene).

According to one embodiment, the plant gene is a coding gene (e.g. protein-coding gene).

According to one embodiment, the plant gene (i.e. exhibiting said predetermined sequence homology to the nucleic acid sequence of the pest gene) does not encode a silencing molecule.

According to one embodiment, the plant gene does not encode for a molecule having an intrinsic silencing activity (e.g. RNA molecule, e.g. non-coding RNA molecule, as discussed in detail below).

According to one embodiment, the plant gene encodes for a molecule having an intrinsic silencing activity (e.g. RNA molecule, e.g. non-coding RNA molecule, as discussed in detail below).

As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission.

According to some embodiments, a pest is an invertebrate pest, including an invertebrate pest which is susceptible to long dsRNA via methods such as, but not limited to, ingestion and/or soaking. Each possibility represents a separate embodiment of the present invention. According to some embodiment, an invertebrate pest which is susceptible to long dsRNA is susceptible to long dsRNA of 26 bp and above, possibly of about 26-50 bp. Each possibility represents a separate embodiment of the present invention.

According to one embodiment, the pest is an invertebrate organism.

Exemplary pests include, but are not limited to, insects, nematodes, snails, slugs, spiders, caterpillars, scorpions, mites, ticks, fungi, and the like.

Insect pests include, but are not limited to, insects selected from the orders Coleoptera (e.g. beetles), Diptera (e.g. flies, mosquitoes), Hymenoptera (e.g, sawflies, wasps, bees, and ants), Lepidoptera (e.g. butterflies and moths), Mallophaga (e.g. lice, e.g. chewing lice, biting lice and bird lice), Hemiptera (e.g. true bugs), Homoptera including suborders Sternorrhyncha (e.g. aphids, whiteflies, and scale insects), Auchenorrhyncha (e.g. cicadas, leafhoppers, treehoppers, planthoppers, and spittlebugs), and Coleorrhyncha (e.g. moss bugs and beetle bugs), Orthroptera (e.g. grasshoppers, locusts and crickets, including katydids and wetas), Thysanoptera (e.g. Thrips), Dermaptera Earwigs), Isoptera (e.g. Termites), Anoplura (e.g. Sucking lice), Siphonaptera (e.g. Flea), Trichoptera caddisflies), etc.

Insect pests of the invention include, but are not limited to, Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, surgarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn flea beetle; Sphenophorus maidis, maize bilibug; Rhopalosiphum maidis, corn leaf aphid; Anuraphis maidiradicis, corn root aphid; Blissus leucopterus leucopterus, chinch bug; Melanoplus femurrubrum, redlegged grasshopper; Melanoplus sanguinipes, migratory grasshopper; Hylemya platura, seedcorn maggot; Agromyza parvicornis, corn blot leafminer; Anaphothrips obscrurus, grass thrips; Solenopsis milesta, thief ant; Tetranychus urticae, twospotted spider mite; Sorghum: Chilo partellus,sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus leucopterus, chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignoseltus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stern maggot; Hylemya coarctate, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge; Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Franklinkiella fusca, tobacco thrips; Tetranychus cinnabarinus, carmine spider mite; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus leucopterus, chinch bug; Acrosternum hilare, green stink bug; Soybean: Pseudoplusia includens, soybean looper; Anticarsia gemmatalis, velvetbean caterpillar; Plathypena scabs, green cloverworm; Ostrinia nubilalis, European corn borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrosternum hilare, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus serous, brown stink bug; Delia platura, seedcorn maggot; Mayetiola destructor, Hessian fly; Petrobia latens, brown wheat mite; Oil Seed Rape: Brevicoryne brassicae, cabbage aphid; Phyllotreta cruciferae, Flea beetle; Mamestra configurata, Bertha armyworm; Plutella xylostella, Diamond-back moth; Delia ssp., Root maggots.

Exemplary nematodes include, but are not limited to, the burrowing nematode (Radopholus similis), Caenorhabditis elegans, Radopholus arabocoffeae, Pratylenchus cofffeae, root-knot nematode (Meloidogyne spp.), cyst nematode (Heterodera and Globodera spp.), root lesion nematode (Pratylenehus spp.), the stem nematode (Ditylenchus dipsaci), the pine wilt nematode (Bursaphelenchus xylophilus), the reniform nematode (Rotylenchulus reniformis), Xiphinema index, Nacobbus aberrans and Aphelenchoides besseyi.

Exemplary fungi include, but are not limited to, Fusarium oxysporum, Leptosphaeria maculans (Phoma lingam), Sclerotinia sclerotiorum, Pyricularia grisea, Gibberella funkuroi (Fusarium moniliforme), Magnaporthe oryzae, Botrvtis cinereal, Puccinia spp., Fusarium graminearum, Blumeria graminis, Mycosphaerella graminicola, Colletotrichum spp., Ustilago maydis, Melampsora lini, Phakopsora pachyrhizi and Rhizoctonia solani.

According to a specific embodiment, the pest is an ant, a termite, a bee, a wasp, a caterpillar, a cricket, a locust, a beetle, a snail, a slug, a nematode, a bug, a fly, a fruitfly, a whitefly, a mosquito, a grasshopper, a planthopper, an earwig, an aphid, a scale, a thrip, a spider, a mite, a psyllid, a tick, a moth, a worm, and a scorpion, in different stages of their lifecycle an ant, a bee, a wasp, a caterpillar, a beetle, a snail, a slug, a nematode, a bug, a fly, a whitefly, a mosquito, a grasshopper, an earwig, an aphid, a scale, a thrip, a spider, a mite, a psyllid, and a scorpion.

According to a specific embodiment, the pest is at any lifecycle stage of its life.

According to one embodiment, the pest is a virus.

The phrase “silencing a pest gene” refers to reducing the level of expression of a polynucleotide or the polypeptide encoded thereby, by at least about 10%, 20%, 30%, 40%, 50%, 60%, 70%, 80%, 90%, 95%, 99% or by 100%, as compared to a pest gene not targeted by the designed long dsRNA molecule of the invention.

Assays for measuring the expression level of a polynucleotide or the polypeptide encoded thereby, include but are not limited to, RT-PCR, Western blot, Immunohistochemistry and/or flow cytometry, sequencing or any other detection methods (as further discussed hereinbelow).

Preferably, silencing of the pest gene results in the suppression, control, and/or killing of the pest which results in limiting the damage that the pest causes to the plant. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant.

The term “pest gene” as used herein refers to any gene in the pest that is essential for growth, development, reproduction or infectivity. The gene may be expressed in any tissue of the pest, however, in specific embodiments, the genes targeted for suppression in the pest are expressed in cells of the gut tissue of the pest, cells in the midgut of the pest, cells lining the gut lumen or the midgut, cells of the pest gut microbiome and cells of the pest immune system. Such target genes can be involved in, for example, gut cell metabolism, growth, differentiation and immune system.

Exemplary pest genes which may be targeted by the present methods include, but are not limited to, the genes listed in Tables 1A-B, hereinbelow.

According to a specific embodiment, the nematode gene comprises the Radophalus similis genes Calreticulin13 (CRT) or collagen 5 (col-5).

According to a specific embodiment, the fungi gene comprises the Fusarium oxysporum genes FOW2, FRP1, and OPR.

According to one embodiment, silencing a pest gene reduces disease symptoms in a plant or reduces damage to the plant (resulting from the pest) by at least about 10%, 20%, 30%, 40%, 50%, 60%, 70%, 80%, 90%, 95%, 99% or by 100%, as compared to a plant harmed by the pest and not being subjected to the designed long dsRNA molecule of the invention.

Assays measuring the control of a pest are commonly known in the art, see, for example, U.S. Pat. No. 5,614,395, herein incorporated by reference. Such techniques include, measuring over time, the average lesion diameter, the pathogen biomass, and the overall percentage of decayed plant tissues. See, for example, Thomma et al. (1998) Plant Biology 95:15107-15111, herein incorporated by reference. See, also Baum et al. (2007) Nature Biotech 11:1322-1326 and WO 2007/035650 which provide both whole plant feeding assays and corn root feeding assays.

According to one embodiment, the method comprises selecting a nucleic acid sequence of a plant gene exhibiting a predetermined sequence homology to a nucleic acid sequence of the pest gene.

According to one embodiment, the sequence homology between the nucleic acid sequence of the plant gene and the nucleic acid sequence of the pest gene comprises 60%-100%, 70%-80%, 70%-90%, 70%-100%, 75%-100%, 80%-90%, 80%-100%, 85%-100%, 90%-100% or 95%-100% identity,

According to a specific embodiment, the sequence homology comprises 75%-100% identity between the nucleic acid sequence of the plant gene and the nucleic acid sequence of the pest gene.

According to a specific embodiment, the sequence homology comprises 85%-100% identity between the nucleic acid sequence of the plant gene and the nucleic acid sequence of the pest gene.

According to a specific embodiment, the sequence homology comprises 75%-100% identity between the nucleic acid sequence of the plant gene and the nucleic acid sequence of the pest gene.

According to one embodiment, the sequence homology comprises at least 50%, 60%, 70%, 75%, 80%, 85%, 90%, 91%, 92%, 93%, 94%, 95%, 96%, 97%, 98%, 99% or 100% identity between the nucleic acid sequence of the plant gene and the nucleic acid sequence of the pest gene.

Homology (e.g., percent homology, sequence identity +sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci, U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., a homology over the entire amino acid or nucleic acid sequences of the invention and not over portions thereof.

The degree of homology or identity between two or more sequences can be determined using various known sequence comparison tools. Following is a non-limiting description of such tools which can be used along with some embodiments of the invention.

When starting with a polynucleotide sequence and comparing to other polynucleotide sequences the EMBOSS-6.0.1 Needleman-Wunsch algorithm (available from emboss(dot)sourceforge(dot)net/apps/cvs/emboss/apps/needle(dot)html) can be used with the following default parameters: (EMBOSS-6.0.1) gapopen=10; gapextend=0.5; datafile=EDNAFULL; brief=YES.

According to some embodiments of the invention, the parameters used with the EMBOSS-6.0.1 Needleman-Wunsch algorithm are gapopen=10; gapextend=0.2; datafile=EDNAFULL; brief=YES.

According to some embodiments of the invention, the threshold used to determine homology using the EMBOSS-6.0.1 Needleman-Wunsch algorithm for comparison of polynucleotides with polynucleotides is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiment, determination of the degree of homology further requires employing the Smith-Waterman algorithm (for protein-protein comparison or nucleotide-nucleotide comparison).

Default parameters for GenCore 6.0 Smith-Waterman algorithm include: model=sw.model.

According to some embodiments of the invention, the threshold used to determine homology using the Smith-Waterman algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiments of the invention, the global homology is performed on sequences which are pre-selected by local homology to the polypeptide or polynucleotide of interest (e.g., 60% identity over 60% of the sequence length), prior to performing the global homology to the polypeptide or polynucleotide of interest (e.g., 80% global homology on the entire sequence). For example, homologous sequences are selected using the BLAST software with the Blastp and tBlastn algorithms as filters for the first stage, and the needle (EMBOSS package) or Frame+algorithm alignment for the second stage. Local identity (Blast alignments) is defined with a very permissive cutoff −60% Identity on a span of 60% of the sequences lengths because it is used only as a filter for the global alignment stage. In this specific embodiment (when the local identity is used), the default filtering of the Blast package is not utilized (by setting the parameter “-F F”).

In the second stage, homologs are defined based on a global identity of at least 80% to the core gene polypeptide sequence. According to some embodiments the homology is a local homology or a local identity.

Local alignments tools include, but are not limited to the BlastP, BlastN, BlastX or TBLASTN software of the National Center of Biotechnology Information (NCBI), ASIA, and the Smith-Waterman algorithm.

According to a specific embodiment, homology is determined using BlastN with parameters: max target sequences=1000, expect threshold=10, word size=11, match score=2, mismatch score=−3, gap existence cost=5, gap extension cost=2.

According to a specific embodiment, selecting a nucleic acid sequence of a plant gene exhibiting a predetermined sequence homology to a nucleic acid sequence of the pest gene is effected by identifying plant transcripts that have “homology stretches” to the pest transcript. According to a specific embodiment, the homology stretch is 20, 21, 22, 23, 24, 25, 26, 27, 28, 29, 30, 35, 40, 41, 42, 43, 44, 45, 46, 47. 48, 49, 50, 100, 150, 200, 250, 300, 400, 500, 600, 700, 800, 900, 1000, 1500, 2000, 2500, 3000, 3500, 4000, 4500, 5000, 5500, 6000, 7000, 8000, 9000, 10,000 or more nucleotides (e.g. 20-50 nucleotides, 20-25 nucleotides, e.g. 21 nucleotides) over the whole plant transcript. Within the 20-50 nucleotides (e.g. 21 nucleotides), the homology of the plant transcript to the pest transcript is preferably 75%, 80%, 85%, 90%, 95%, 99% or 100%.

According to a specific embodiment, when the pest is a nematode (Heterodera glycines), the pest gene is as set forth in accession no. AF469060.1 (Heterodera glycines ubiquitin extension protein), the plant gene is as set forth in NM_001203752.2 (Arabidopsis thaliana ubiquitin 11 (UBQ11)).

According to a specific embodiment, when the pest is a nematode (Heterodera glycines), the pest gene is as set forth in accession no. AF500024.1 (Heterodera glycines putative gland protein G8H07), the plant gene is as set forth in NM_116351.7 (Arabidopsis thaliana glycosyl transferase family 1 protein (AT4G01210)).

According to a specific embodiment, when the pest is a nematode (Heterodera glycines), the pest gene is as set forth in accession no. AF502391.1 (Heterodera glycines putative gland protein G10A06), the plant gene is as set forth in NM_001037071.1 (Arabidopsis thaliana bZIP transcription factor family protein (TGA1)).

According to one embodiment, the method comprises modifying a plant endogenous nucleic acid sequence encoding an RNA molecule so as to impart silencing specificity towards the plant gene, such that small RNA molecules capable of recruiting RNA-dependent RNA Polymerase (RdRp) processed from the RNA molecule form base complementation with a transcript of the plant gene to produce the long dsRNA molecule capable of silencing the pest gene.

According to one embodiment, the RNA molecule is a non-coding RNA molecule.

As used herein, the term “non-coding RNA molecule” refers to a RNA sequence that is not translated into an amino acid sequence and does not encode a protein.

According to one embodiment, the nucleic acid sequence encoding the RNA molecule is positioned in a non-coding gene (e.g. non-protein coding gene). Exemplary non-coding parts of the genome include, but are not limited to, introns, genes of non-coding RNAs, DNA methylation regions, enhancers and locus control regions, insulators, S/MAR sequences, non-protein-coding pseudogenes, transposons, non-autonomous transposable elements (e.g. Alu, SINES and mutated non-coding transposons and retrotransposons) and simple repeats of centromeric and telomeric regions of chromosomes.

According to one embodiment, the nucleic acid sequence encoding the RNA molecule is positioned in a non-coding gene that is ubiquitously expressed.

According to one embodiment, the nucleic acid sequence encoding the RNA molecule is positioned in a non-coding gene that is expressed in a tissue-specific manner (e.g. in a leaf, fruit or flower).

According to one embodiment, the nucleic acid sequence encoding the RNA molecule is positioned in a non-coding gene that it is expressed in an inducible manner.

According to one embodiment, the nucleic acid sequence encoding the RNA molecule is positioned in a non-coding gene that it is developmentally regulated.

According to one embodiment, the nucleic acid sequence encoding the RNA molecule is positioned between genes, i.e. intergenic region.

According to one embodiment, the nucleic acid sequence encoding the RNA molecule is positioned within an intron of a non-coding gene.

According to one embodiment, the nucleic acid sequence encoding the RNA molecule is positioned in a coding gene (e.g. protein-coding gene).

According to one embodiment, the nucleic acid sequence encoding the RNA molecule is positioned within an exon of a coding gene (e.g. protein-coding gene).

According to one embodiment, the nucleic acid sequence encoding the RNA molecule is positioned within an exon encoding an untranslated region (UTR) of a coding gene (e.g. protein-coding gene).

According to one embodiment, the nucleic acid sequence encoding the RNA molecule is positioned within a translated exon of a coding gene (e.g. protein-coding gene).

According to one embodiment, the nucleic acid sequence encoding the RNA molecule is positioned within an intron of a coding gene (e.g. protein-coding gene).

According to one embodiment, the nucleic acid sequence encoding the RNA molecule is positioned within a coding gene that is ubiquitously expressed.

According to one embodiment, the nucleic acid sequence encoding the RNA molecule is positioned within a coding gene that is expressed in a tissue-specific manner (e.g. in a leaf, fruit or flower).

According to one embodiment, the nucleic acid sequence encoding the RNA molecule is positioned within coding gene that it is expressed in an inducible manner.

According to one embodiment, the nucleic acid sequence encoding the RNA molecule is positioned in a coding gene that it is developmentally regulated.

According to one embodiment, the RNA molecule (e.g. non-coding RNA molecule) is typically subject to the RNA silencing processing mechanism or activity. However, also contemplated herein are a few changes in nucleotides (e.g, for miRNA up to 24 nucleotides) which may elicit a processing mechanism that results in recruitment of RdRP, in RNA interference or in translation inhibition.

According to a specific embodiment, the RNA molecule is endogenous (naturally occurring, e.g. native) to the plant cell. It will be appreciated that the RNA molecule can also be exogenous to the cell (i.e. externally added and which is not naturally occurring in the plant cell).

According to some embodiments, the RNA molecule (e.g. non-coding RNA molecule) comprises an intrinsic translational inhibition activity.

According to some embodiments, the RNA molecule (e.g. non-coding RNA molecule) comprises an intrinsic RNA interference (RNAi) activity.

According to some embodiments, the RNA molecule (e.g. non-coding RNA molecule) does not comprise an intrinsic translational inhibition activity or an intrinsic RNAi activity (i.e. the non-coding RNA molecule does not have an RNA silencing activity).

According to an embodiment of the invention, the RNA molecule (e.g, non-coding RNA molecule) is specific to a native plant RNA (e.g., a natural plant RNA) and does not cross inhibit or silence a pest RNA or plant RNA of interest (i.e. a transcript of the plant gene) unless designed to do so (as discussed below) exhibiting 100% or less global homology to the target gene, e.g., less than 99%, 98%, 97%, 96%, 95%, 94%, 93%, 92%, 91%, 90%, 89%, 88%, 87%, 86%, 85%, 84%, 83%, 82%, 81% global homology to the target gene; as determined at the RNA or protein level by RT-PCR, Western blot, immunohistochemistry and/or flow cytometry, sequencing or any other detection methods.

According to one embodiment, the RNA molecule (e.g. non-coding RNA molecule) is a RNA silencing or RNA interference (RNAi) molecule (also referred to as a “silencing molecule”).

The term “RNA silencing” or RNAi refers to a cellular regulatory mechanism in which non-coding RNA molecules (the “RNA silencing molecule”, “silencing molecule” or “RNAi molecule”) mediate, in a sequence specific manner, co- or post-transcriptional inhibition of gene expression or translation.

As used herein, a “silencing molecule capable of recruiting RNA-dependent RNA Polymerase (RdRp)” refers to a silencing molecule which is able to engage RdRp to the site of its interaction with the target transcript, thus enabling the formation of a long-dsRNA based on another RNA molecule as a template. In a non-limiting example, the silencing molecule capable of recruiting RdRp is a miRNA, such as, but not limited to, a miRNA of 22 nt length, and a TAS transcript serves as a template for the miRNA/RISC/RdRp complex, thus resulting in a long dsRNA based on the TAS transcript.

According to one embodiment, the RNA molecule (e.g. RNA silencing molecule) is capable of mediating RNA repression during transcription (co-transcriptional gene silencing).

According to a specific embodiment, co-transcriptional gene silencing includes epigenetic silencing (e.g. chromatic state that prevents functional gene expression).

According to one embodiment, the RNA molecule (e.g. RNA silencing molecule) is capable of mediating RNA repression after transcription (post-transcriptional gene silencing).

Post-transcriptional gene silencing (PIGS) typically refers to the process (typically occurring in the cell cytoplasm) of degradation or cleavage of messenger RNA (mRNA) molecules which decrease their activity by preventing translation. For example, and as discussed in detail below, a guide strand of a RNA silencing molecule pairs with a complementary sequence in a mRNA molecule and induces cleavage by e.g. Argonaute 2 (Ago2).

Co-transcriptional gene silencing typically refers to inactivation of gene activity (i.e. transcription repression) and typically occurs in the cell nucleus. Such gene activity repression is mediated by epigenetic-related factors, such as e.g. methyl-transferases, that methylate target DNA and histones. Thus, in co-transcriptional gene silencing, the association of a small RNA with a target RNA (small RNA-transcript interaction) destabilizes the target nascent transcript and recruits DNA- and histone-modifying enzymes (i.e. epigenetic factors) that induce chromatin remodeling into a structure that repress gene activity and transcription. Also, in co-transcriptional gene silencing, chromatin-associated long non-coding RNA scaffolds may recruit chromatin-modifying complexes independently of small RNAs. These co-transcriptional silencing mechanisms form RNA surveillance systems that detect and silence inappropriate transcription events, and provide a memory of these events via self-reinforcing epigenetic loops [as described in D. Hoch and. D. Moazed, RNA-mediated epigenetic regulation of gene expression, Nat Rev Genet. (2015) 16(2): 71-84].

According to an embodiment of the invention, the RNAi biogenesis/processing machinery generates the RNA silencing molecule.

According to an embodiment of the invention, the RNAi biogenesis/processing machinery generates the RNA silencing molecule, but no specific target has been identified.

According to one embodiment, the RNA molecule (e.g. non-coding RNA molecule) is a capable of inducing RNA interference (RNAi.).

According to one embodiment, the RNA molecule (e.g. non-coding RNA molecule or the RNA silencing molecule) is processed from a precursor.

According to one embodiment, the RNA molecule (e.g. non-coding RNA molecule or the RNA silencing molecule) is processed from a single stranded RNA (ssRNA) precursor.

According to one embodiment, the RNA molecule (e.g. non-coding RNA molecule or the RNA silencing molecule) is processed from a duplex-structured single-stranded RNA precursor.

According to one embodiment, the RNA molecule (e.g. non-coding RNA molecule or the RNA silencing molecule) is processed from a dsRNA precursor (e.g. comprising perfect and imperfect base pairing).

According to one embodiment, the RNA molecule (e.g. non-coding RNA molecule or the RNA silencing molecule) is processed from a non-structured RNA precursor.

According to one embodiment, the RNA molecule (e.g. non-coding RNA molecule or the RNA silencing molecule) is processed from a protein-coding RNA precursor.

According to one embodiment, the RNA molecule (e.g. non-coding RNA molecule or the RNA silencing molecule) is processed from a RNA precursor.

According to one embodiment, the RNA molecule (e.g. non-coding RNA molecule or the RNA silencing molecule) is processed and engaged with RNA-induced silencing complex (RISC).

According to one embodiment, the RNA molecule (e.g. non-coding RNA molecule or the RNA silencing molecule) is processed and engaged with RNAi processing machinery such as, for example, with ribonucleases, including but not limited to, Dicer, Ago2, the DICER protein family (e.g. DCR1 and DCR2), DICER-LIKE protein family (e.g. DCL1, DCL2, DCL3, DCL4), ARGONAUTE protein family (e.g. AGO1, AGO2, AGO3, AGO4), tRNA cleavage enzymes (e.g. RNY1,ANGIOGENIN, RNase P, RNase P-like, SLFN3, ELAC1 and ELAC2), and Piwi-interacting RNA (piRNA) related proteins (e.g. AGO3, AUBERGINE, HIWI, HIWI2, HIWI3, PIWI, ALG1 and ALG2) (as further discussed below).

According to one embodiment, the dsRNA can be derived from two different complementary RNAs, or from a single RNA that folds on itself to form dsRNA.

Following is a detailed description of RNA silencing molecules (e.g. non-coding RNA molecules) which are engaged with RNA-induced silencing complex (RISC) and comprise an intrinsic RNAi activity (e.g. are RNA silencing molecules) that can be used according to specific embodiments of the present invention.

Poled and imperfect based paired RNA (i.e. double stranded RNA; dsRNA), siRNA and shRNA—The presence of long dsRNAs in cells stimulates the activity of a ribonuclease III enzyme referred to as dicer. Dicer (also known as endoribonuclease Dicer or helicase with RNase motif) is an enzyme that in plants is typically referred to as Dicer-like (DCL) protein. Different plants have different numbers of DCL genes, thus for example, Arabidopsis genome typically has four DCL genes, rice has eight DCL genes, and maize genome has five DCL genes. Dicer is involved in the processing of the dsRNA into short pieces of dsRNA known as short interfering RNAs (siRNAs). siRNAs derived from dicer activity are typically about 21 to about 23 nucleotides in length and comprise about 19 base pair duplexes with two 3′ nucleotides overhangs.

According to one embodiment dsRNA precursors longer than 21 bp are used. Various studies demonstrate that long dsRNAs can be used to silence gene expression without inducing the stress response or causing significant off-target effects—see for example [Surat et al., Nucleic Acids Research, 2006, Vol. 34, No. 13 3803-3810; Bhargava. A et al. Brain Res. Protoc. 2004;13:115-125; Diallo M., et al., Oligonucleotides. 2003;13:381-392; Paddison P. J., et al., Proc. Natl Acad, Sci, USA. 2002;99:1443-1448; Tran N., et al., FEBS Lett, 2004;573:127-134].

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position, but not the composition, of the 3′-overhang influences potency of a siRNA and asymmetric duplexes having a 3′-overhang on the antisense strand are generally more potent than those with the 3′-overhang on the sense strand (Rose et al., 2005).

The strands of a double-stranded interfering RNA (e.g., a siRNA) may be connected to form a hairpin or stem-loop structure (e.g., a shRNA). Thus, as mentioned, the RNA silencing molecule of some embodiments of the invention may also be a short hairpin RNA (shRNA).

The term short hairpin RNA, “shRNA”, as used herein, refers to a RNA molecule having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

The RNA silencing molecule of some embodiments of the invention need not be limited to those molecules containing only RNA, but further encompasses chemically-modified nucleotides and non-nucleotides.

Various types of siRNAs are contemplated by the present invention, including trans-acting siRNAs (TasiRNAs), repeat-associated siRNAs (Ra-siRNAs) and natural-antisense transcript-derived siRNAs (Nat-siRNAs).

According to a specific embodiment, the RNA molecule (e.g. non-coding RNA molecule) is a phased small interfering RNA (phasRNA) “PhasiRNAs” are derived from an mRNA converted to dsRNA by RDR6 and processed by DCL4, exemplified by the category of Arabidopsis trans-acting siRNAs (tasiRNAs) (Vazquez et al., 2004). In an exceptional case, phasiRNAs may also be 24-nucleotide products of DCL5 (previously known as DCL3b) in grass reproductive tissues (Song et al., 2012). The trans-acting name (tasiRNAs) of some phasiRNAs comes from their ability to function like miRNAs in a homology-dependent manner, directing AGO1-dependent slicing of mRNAs from genes other than that of their source mRNA (see below).

According to a specific embodiment, the RNA molecule (e.g. non-coding RNA molecule) is a tasiRNA. “TasiRNA” are a class of secondary siRNAs generated from noncoding TAS transcripts by miRNA triggers in a phased pattern (Peragine et al., 2004; Vazquez et al., 2004; Allen et al., 2005; Yoshikawa et al., 2005). The term “phased” indicates simply that the small RNAs are generated precisely in a head-to-tail arrangement, starting from a specific nucleotide; this arrangement results from miRNA-triggered initiation followed by DCL4-catalyzed cleavage. The primary proteins that participate in tasiRNA biogenesis include, but are not limited to, RDR6, SUPPRESSOR OF GENE SILENCING3 (SGS3), AGO1, AGO7, and DOUBLE-STRANDED RNA BINDING FACTOR4 (Peragine et al., 2004; Vazquez et al., 2004; Xie et al., 2005; Adenot et al., 2006; Montgomery et al., 2008a; Fukudome et al., 2011). Most importantly, there are two mechanisms by which 21-nucleotide tasiRNAs are produced, known as the “one-hit” or “two-hit” pathways. In the one-hit mechanism, a single miRNA directs cleavage of the mRNA target triggering the production of phasiRNAs in the fragment 39 to (or downstream of) the target site (Allen et al., 2005). The one-hit miRNA trigger is typically 22 nucleotides in length (Chen et al., 2010; Cuperus et al., 2010). In the two-hit model, a pair of 21-nucleotide miRNA target sites is employed, of which cleavage occurs at only the 39 target site, triggering the production of phasiRNAs fragment (or upstream of) the target site (Axtell et al., 2006).

According to one embodiment, silencing RNA includes “piRNA” which is a class of Piwi-interacting RNAs of about 26 and 31 nucleotides in length. piRNAs typically form RNA-protein complexes through interactions with Piwi proteins, i.e. antisense piRNAs are typically loaded into Piwi proteins (e.g. Piwi, Ago3 and Aubergine (Aub)).

miRNA—According to another embodiment the RNA silencing molecule may be a miRNA.

The term “microRNA”, “miRNA”, and “miR” are synonymous and refer to a collection of non-coding single-stranded RNA molecules of about 19-24 nucleotides in length, which regulate gene expression, miRNAs are found in a wide range of organisms (e.g. insects, mammals, plants, nematodes) and have been shown to play a role in development, homeostasis, and disease etiology.

Initially the pre-miRNA is present as a long non-perfect double-stranded stern loop RNA that is further processed by Dicer into a siRNA-like duplex, comprising the mature guide strand (miRNA) and a similar-sized fragment known as the passenger strand (miRNA*). The miRNA and miRNA* may be derived from opposing arms of the pri-miRNA and pre-miRNA. miRNA* sequences may be found in libraries of cloned miRNAs but typically at lower frequency than the miRNAs as it is, in most cases, not functional and degraded in the cell.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-8 of the miRNA (referred as “seed sequence”).

A number of studies have looked at the base-pairing requirement between miRNA and its mRNA target for achieving efficient inhibition of translation (reviewed by Bartel 2004, Cell 116-281). Computational studies, analyzing miRNA binding on whole genomes have suggested a specific role for bases 2-8 at the 5′ of the miRNA (also referred to as “seed sequence”) in target binding but the role of the first nucleotide, found usually to be “A” was also recognized (Lewis et al. 2005 Cell 120-15). Similarly, nucleotides 1-7 or 2-8 were used to identify and validate targets by Krek et al. (2005, Nat Genet 37-495). The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stern strands may lead to a population of different hairpin structures. Variability in the stem structures may also lead to variability in the products of cleavage by Drosha and Dicer.

According to one embodiment, miRNAs can be processed independently of Dicer, e.g. by Argonaute 2.

It will be appreciated that the pre-miRNA sequence may comprise from 45-90, 60-80 or 60-70 nucleotides while the pri-miRNA sequence may comprise from 45-30,000, 50-25,000, 100-20,000, 1,000-1,500 or 80-100 nucleotides,

Antisense Antisense is a single stranded RNA designed to prevent or inhibit expression of a gene by specifically hybridizing to its mRNA. Downregulation of a target RNA can be effected using an antisense polynucleotide capable of specifically hybridizing with an mRNA transcript encoding the target RNA.

Transposable Element RNA

Transposable genetic elements (TEs) comprise a vast array of DNA sequences, all having the ability to move to new sites in genomes either directly by a cut-and-paste mechanism (transposons) or indirectly through an RNA intermediate (retrotransposons). TEs are divided into autonomous and non-autonomous classes depending on whether they have ORFs that encode proteins required for transposition. RNA-mediated gene silencing is one of the mechanisms in which the genome control TEs activity and deleterious effects derived from genome genetic and epigenetic instability.

As mentioned, the RNA molecule (e.g. non-coding RNA molecule) may not comprise a canonical (intrinsic) RNAi activity (e.g. is not a canonical RNA silencing molecule, or its target has not been identified). Such non-coding RNA molecules include the following:

According to one embodiment, the RNA molecule (e.g. non-coding RNA molecule) is a transfer RNA (tRNA). The term “tRNA” refers to a RNA molecule that serves as the physical link between nucleotide sequence of nucleic acids and the amino acid sequence of proteins, formerly referred to as soluble RNA or sRNA. tRNA is typically about 76 to 90 nucleotides in length.

According to one embodiment, the RNA molecule (e.g. non-coding RNA molecule) is a ribosomal RNA (rRNA). The term “rRNA” refers to the RNA component of the ribosome i.e. of either the small ribosomal subunit or the large ribosomal subunit.

According to one embodiment, the RNA molecule (e.g. non-coding RNA molecule) is a small nuclear RNA (snRNA or U-RNA). The terms “sRNA” or “U-RNA” refer to the small RNA molecules found within the splicing speckles and Cajal bodies of the cell nucleus in eukaryotic cells. snRNA is typically about 150 nucleotides in length.

According to one embodiment, the RNA molecule (e.g, non-coding RNA molecule) is a small nucleolar RNA (snoRNA). The term “snoRNA” refers to the class of small RNA molecules that primarily guide chemical modifications of other RNAs, e.g. rRNAs, tRNAs and snRNAs. snoRNA is typically classified into one of two classes: the C/D box snoRNAs are typically about 70-120 nucleotides in length and are associated with methylation, and the H/ACA box snoRNAs are typically about 100-200 nucleotides in length and are associated with pseudouridylation.

Similar to snoRNAs are the scaRNAs (i.e. Small Cajal body RNA genes) which perform a similar role in RNA maturation to snoRNAs, but their targets are spliceosomal snRNAs and they perform site-specific modifications of spliceosomal snRNA precursors (in the Cajal bodies of the nucleus).

According to one embodiment, the RNA molecule (e.g. non-coding RNA molecule) is an extracellular RNA (exRNA). The term “exRNA” refers to RNA species present outside of the cells from which they were transcribed (e.g. exosomal RNA).

According to one embodiment, the RNA molecule non-coding RNA molecule) is a repeat-derived RNA. The term “repeat-derived RNA” refers to an RNA encoded by DNA derived from inverted genomic repeats (such as, but not limited to, DNA generated by DNA recombination, genomic loci duplication, transposition events etc).

According to one embodiment, the RNA molecule (e.g. non-coding RNA molecule) is a long non-coding RNA (lncRNA). The term “lncRNA” or “long ncRNA” refers to non-protein coding transcripts typically longer than 200 nucleotides.

According to a specific embodiment, non-limiting examples of RNA molecules (e.g. non-coding RNA molecules) engaged with RISC include, but are not limited to, microRNA (miRNA), piwi-interacting RNA (piRNA), short interfering RNA (siRNA), short-hairpin RNA (shRNA), phased small interfering RNA (phasiRNA), trans-acting siRNA (tasiRNA), small nuclear RNA (snRNA or URNA), transposable element RNA (e.g. autonomous and non-autonomous transposable RNA), transfer RNA (tRNA), small nucleolar RNA (snoRNA), Small Cajal body RNA (scaRNA), ribosomal RNA (rRNA), extracellular RNA (exRNA), repeat-derived RNA, and long non-coding RNA (lncRNA).

According to a specific embodiment, non-limiting examples of RNAi molecules engaged with RISC include, but are not limited to, small interfering RNA (siRNA), short hairpin RNA (shRNA), microRNA (miRNA), Wiwi-interacting RNA (piRNA), phased small interfering RNA (phasiRNA), and trans-acting siRNA (tasiRNA).

According to one embodiment, small RNA molecules processed from the RNA molecule (e.g. non-coding RNA molecule) of some embodiments of the invention are capable of recruiting RNA-dependent RNA Polymerase (RdRp).

The terms “processed” refer to the biogenesis by which RNA molecules are cleaved into small RNA form capable of engaging with RNA-induced silencing complex (RISC). For example, pre-miRNA is processed into a mature miRNA e.g. by Dicer.

As used herein, the term “small RNA form” or “small RNAs” or “small RNA molecules” refers to the mature small RNA being capable of hybridizing with a target RNA e.g. transcript of the plant gene (or fragment thereof).

According to one embodiment, the small RNAs comprise no more than 250 nucleotides in length, e.g. comprise 20-250, 20-200, 20-150, 20-100, 20-50, 20-40, 20-30, 20-25, 20-26, 30-100, 30-80, 30-60, 30-50, 30-40, 50-150, 50-100, 50-80, 50-70, 100-250, 100-200, 100-150, 150-250, 150-200 nucleotides.

According to a specific embodiment, the small RNA molecules comprise 20-50 nucleotides.

According to a specific embodiment, the small RNA molecules comprise 20-30 nucleotides.

According to a specific embodiment, the small RNA molecules comprise 21-29 nucleotides.

According to a specific embodiment, the small RNA molecules comprise 21-24 nucleotides.

According to a specific embodiment, the small RNA molecules comprise 21 nucleotides.

According to a specific embodiment, the small RNA molecules comprise 22 nucleotides.

According to a specific embodiment, the small RNA molecules comprise 23 nucleotides.

According to a specific embodiment, the small RNA molecules comprise 24 nucleotides.

According to a specific embodiment, the small RNA molecules consist of 20-50 nucleotides.

According to a specific embodiment, the small RNA molecules consist of 20-30 nucleotides.

According to a specific embodiment, the small RNA molecules consist of 21-29 nucleotides.

According to a specific embodiment, the small RNA molecules consist of 21-24 nucleotides.

According to a specific embodiment, the small RNA molecules consist of 21 nucleotides.

According to a specific embodiment, the small RNA molecules consist of 22 nucleotides.

According to a specific embodiment, the small RNA molecules consist of 23 nucleotides.

According to a specific embodiment, the small RNA molecules consist of 24 nucleotides.

According to one embodiment, the small RNA molecules comprise a silencing activity (i.e. are silencing molecules).

As mentioned, silencing molecules (e.g. RNA silencing molecules) of some embodiments of the invention are capable of recruiting RNA-dependent RNA Polymerase (RdRp).

The term “RNA-dependent RNA Polymerase” or “RdRp” refers to the enzyme that catalyzes the replication of RNA from an RNA template.

According to one embodiment, the small RNA molecule comprises an amplifier or primer activity towards the RdRp.

According to a specific embodiment, the silencing molecule capable of recruiting the RdRp is selected from microRNA (miRNA), small interfering RNA (siRNA), short hairpin RNA (shRNA), Piwi-interacting RNA (piRNA), trans-acting siRNA (tasiRNA), phased small interfering RNA (phasiRNA), transfer RNA (tRNA), small nuclear RNA (snRNA), ribosomal RNA (rRNA), small nucleolar RNA (snoRNA), extracellular RNA (exRNA), a repeat-derived RNA, autonomous and non-autonomous transposable RNA,

According to some embodiments of the invention, the silencing molecule capable of recruiting the RdRp comprises 21-24 nucleotides.

According to some embodiments of the invention, the silencing molecule capable of recruiting the RdRp comprises 21 nucleotides.

According to some embodiments of the invention, the silencing molecule capable of recruiting the RdRp comprises 22 nucleotides.

According to some embodiments of the invention, the silencing molecule capable of recruiting the RdRp comprises 23 nucleotides.

According to some embodiments of the invention, the silencing molecule capable of recruiting the RdRp comprises 24 nucleotides.

According to some embodiments of the invention, the silencing molecule capably: of recruiting the RdRp consists of 21 nucleotides.

According to some embodiments of the invention, the silencing molecule capable of recruiting the RdRp consists of 22 nucleotides,

According to some embodiments of the invention, the silencing molecule capable of recruiting the RdRp consists of 23 nucleotides.

According to some embodiments of the invention, the silencing molecule capable of recruiting the RdRp consists of 24 nucleotides,

According to a specific embodiment, the silencing molecule capable of recruiting the RdRp is miRNA.

According to a specific embodiment, the miRNA comprises a 21-25 nucleotides mature small RNA

According to a specific embodiment, the miRNA comprises a 21 nucleotides mature small RNA.

According to a specific embodiment, the miRNA comprises a 22 nucleotides mature small RNA.

According to a specific embodiment, the miRNA comprises a 23 nucleotides mature small RNA.

According to a specific embodiment, the miRNA comprises a 24 nucleotides mature small RNA.

According to a specific embodiment, the miRNA comprises a 25 nucleotides mature small RNA.

According to a specific embodiment, the miRNA is a 21-25 nucleotides mature small RNA.

According to a specific embodiment, the miRNA is a 21 nucleotides mature small RNA.

According to a specific embodiment, the miRNA comprises a 22 nucleotides mature small RNA.

According to a specific embodiment, the miRNA is a 23 nucleotides mature small RNA.

According to a specific embodiment, the miRNA is a 24 nucleotides mature small RNA.

According to a specific embodiment, the miRNA is a 25 nucleotides mature small RNA.

Exemplary miRNA include, but are not limited to, miR-156a, miR-156c, miR-162a, miR-162b, miR-167d, miR-169b, miR-173, miR-393a, miR-393b, miR-402, miR-403, miR-447a, miR-447b, miR-447c, miR-472, miR-771, miR-777, miR-828, miR-830, miR-831, miR-831, miR-833a, miR-833a, miR-840, miR-845b, miR-848, miR-850, miR-853, miR-855, miR-856, miR-864, miR-2933a, miR-2933b, miR-2936, miR-4221, miR-5024, miR-5629, miR-5648, miR-5996, miR-8166, miR-8167a, miR-8167b, miR-8167c, miR-8167d, miR-8167e, miR-8167f, miR-8177, and miR-8182.

As mentioned above, the method of some embodiments of the invention comprises modifying a plant endogenous nucleic acid sequence encoding an RNA molecule so as to impart silencing specificity towards the plant gene.

According to one embodiment, when the RNA molecule does not have an intrinsic silencing activity the method further comprises introducing into the plant cell a DNA editing agent conferring a silencing specificity of the RNA molecule towards the plant gene.

According to one embodiment, when the RNA molecule has an intrinsic silencing activity towards a native plant gene, the method further comprises introducing into the plant cell a DNA editing agent which redirects a silencing specificity of the RNA molecule towards the plant gene, the plant gene and the native plant gene being distinct.

Methods of modifying nucleic acid sequences are discussed in detail hereinbelow.

According to some embodiments, e.g. the second model described herein, a nucleic acid sequence of a plant gene is modified so that is encodes a long dsRNA. molecule which imparts a silencing specificity towards a pest gene. According to some embodiments, this nucleic acid sequence encodes an RNA molecule which has an intrinsic silencing activity towards a native plant gene, such that this modification results with a silencing RNA having a novel silencing activity (e.g. towards a pest gene) in addition or instead to the intrinsic silencing activity. Each possibility represents a separate embodiment of the present invention.

Thus, according to another aspect of the present invention there is provided a method of producing a long dsRNA molecule in a plant cell that is capable of silencing a pest gene, the method comprising:

(a) selecting in a genome of a plant a nucleic acid sequence encoding a silencing molecule having a plant gene as a target, the silencing molecule capable of recruiting RNA-dependent RNA Polymerase (RdRp);

(b) modifying a nucleic acid sequence of the plant gene so as to impart a silencing specificity towards the pest gene, such that a transcript of the plant gene comprising the silencing specificity forms base complementation with the silencing molecule capable of recruiting the RdRp to produce the long dsRNA molecule capable of silencing the pest gene,

thereby producing the long dsRNA molecule in the plant cell that is capable of silencing the pest gene.

According to one embodiment, the plant gene does not encode for a molecule having an intrinsic silencing activity.

According to one embodiment, when the plant gene does not encode for a molecule having an intrinsic silencing activity, the method further comprises introducing into the plant cell a DNA editing agent conferring a silencing specificity of the plant gene towards the pest gene.

According to one embodiment, the plant gene encodes for a molecule having an intrinsic silencing activity towards a native plant gene.

According to one embodiment, the plant gene having an intrinsic silencing activity is selected from a microRNA (miRNA), a small interfering RNA (siRNA), a short hairpin RNA (shRNA), a Piwi-interacting RNA (piRNA), a trans-acting siRNA (tasiRNA), a phased small interfering RNA (phasiRNA), a transfer RNA (tRNA), a small nuclear RNA snRNA), a ribosomal RNA (rRNA), a small nucleolar RNA (snoRNA), an extracellular RNA (exRNA), a repeat-derived RNA, an autonomous and a non-autonomous transposable RNA.

According to some embodiments, the plant gene encoding for an RNA having the intrinsic silencing activity encodes for a phased secondary siRNA-producing molecules.

As used herein, the phrase “phased secondary siRNA-producing molecule” refers to an RNA transcript which is capable of forming base complementation with a primary silencing molecule (e.g a miRNA) which recruits an RNA dependent RNA polymerase (RdRp), thus being transcribed into a long dsRNA molecule that is, in turn, processed to secondary silencing RNA molecules (i.e. phased RNAs). According to some embodiments, the phased secondary siRNA-producing molecule is selected from the group consisting of a tasiRNA and a phasiRNA.

According to some embodiments, the phased secondary siRNA-producing molecule is capable of being processed to a plurality of secondary silencing RNA molecules, i.e. at least two secondary silencing RNA molecules. According to some embodiments, modifying the gene encoding the phased secondary siRNA-producing molecule comprises modifying only part of the secondary silencing RNA molecules formed by processing of this phased secondary siRNA-producing molecule. According to a particular embodiment, modifying the gene encoding the phased secondary siRNA-producing molecule comprises modifying only one secondary silencing RNA molecules formed by processing of this phased secondary siRNA-producing molecule. According to some embodiments, modifying the gene encoding the phased secondary siRNA-producing molecule comprises modifying at least one secondary silencing RNA molecules formed by processing of this phased secondary siRNA-producing molecule. According to other embodiments, modifying the gene encoding the phased secondary siRNA-producing molecule comprises modifying all the secondary silencing RNA molecules formed by processing of this phased secondary siRNA-producing molecule. Without wishing to be bound by theory or mechanism, modifying a gene encoding a phased secondary siRNA-producing molecule such that the silencing specificity of only one of the secondary silencing RNA molecules is directed towards a new target (e.g. a pest RNA) is sufficient to induce at least partial silencing of this new target.

According to some embodiments, the length of the secondary silencing RNA molecule sequence to be modified is the length of secondary silencing molecules within the pest of target (e.g. if a tasiRNA is processed within a pest such that 24 nt secondary sRNAs are formed, the sequence of the gene encoding the phased secondary siRNA-producing molecule in a plant cell is modified such that at least one 24 nt sequence targets the pest RNA of choice). According to some embodiments, modifying a nucleic acid sequence of the plant gene (e.g. a plant gene encoding a phased secondary siRNA-producing molecule) so as to impart a silencing specificity towards a pest gene comprises modifying a sequence of 21-30 nt, optionally 2.4 nt, possibly 30 nt in the plant gene, so that the encoded sequence is substantially complementary to an RNA encoded by the pest gene. Each possibility represents a separate embodiment of the present invention. Without wishing to be bound by theory or mechanism, modifying a gene encoding a phased secondary siRNA-producing molecule such that 30 nt of the encoded sequence are complementary to the pest gene ensures that processing of the long dsRNA (which might be different than the processing within the plant gene) results in secondary RNA molecules with a functional silencing activity in the pest.

According to a specific embodiment, the plant gene having the intrinsic silencing activity is a trans-acting-siRNA-producing (TAS) molecule.

According to a specific embodiment, the plant gene comprises a binding site for the silencing molecule.

According to a specific embodiment, the plant gene comprises a binding site for the miRNA molecule.

According to a specific embodiment, the miRNA includes, but is not limited to, miR-156a, miR-156c, miR-162a, miR-162b, miR-167d, miR-169b, miR-393a, miR-393b, miR-402, miR-403, miR-447a, miR-447b, miR-447c, miR-472, miR-771, miR-777, miR-828, miR-830, miR-831, miR-831, miR-833a, miR-833a, miR-840, miR-845b, miR-848, miR-850 miR-853, miR-855, miR-856, miR-864, miR-2933a, miR-2933b, miR-2936, miR-4221, miR-5024, miR-5629, miR-5648, miR-5996, miR-8166, miR-8167a, miR-8167b, miR-8167c, miR-8167d, miR-8167e, miR-8167f, miR-8177, and miR-8182.

According to one embodiment, when the plant gene encodes for a molecule having an intrinsic silencing activity, the method further comprises introducing into the plant cell a DNA editing agent which redirects a silencing specificity of the plant gene towards the pest gene, the pest gene and the native plant gene being distinct.

As used herein, the term “redirects a silencing specificity” refers to reprogramming the original specificity of the RNA molecule or the transcript of the plant gene towards a non-natural target of the RNA molecule or the transcript of the plant gene. Accordingly, the original specificity of the RNA molecule or the transcript of the plant gene is abolished (i.e. loss of function) and the new specificity is towards a target distinct of the natural target (i.e. RNA of a plant or a pest, respectively), i.e., gain of function. It will be appreciated that only gain of function occurs in cases that the RNA molecule or the transcript of the plant gene has no intrinsic silencing activity.

As used herein, the term “native plant RNA” refers to a RNA sequence naturally bound by a RNA molecule (e.g. non-coding RNA molecule, e.g. silencing molecule). Thus, the native plant RNA (i.e. transcript of a native plant gene) is considered by the skilled artisan as a natural substrate (i.e. target) for the RNA molecule (e.g. non-coding RNA, e.g. silencing molecule).

As used herein, the term “plant RNA” or “plant target RNA” refers to a RNA sequence (coding or non-coding) not naturally bound by a RNA molecule (e.g. non-coding RNA, e.g. silencing molecule). Thus, the plant RNA (i.e. transcript of a plant gene) is not a natural substrate (i.e. target) of the RNA molecule (e.g. non-coding RNA, e.g. silencing molecule).

As used herein, the term “pest RNA” or “pest target RNA” refers to a RNA sequence to be silenced by the designed plant RNA and/or by the generated dsRNA molecules and secondary small RNAs (generated by processing of the dsRNA). Thus, the pest RNA (i.e. transcript of a pest gene) is not a natural substrate (i.e. target) of the plant RNA or the dsRNA or the secondary small molecules.

As used herein, the phrase “silencing a gene” refers to the absence or observable reduction in the level of mRNA and/or protein products from the target gene (e.g. due to co- and/or post-transcriptional gene silencing). Thus, silencing of a target gene can be by 5%, 10%, 20%, 30%, 40%, 50%, 60%, 70%, 80%, 90%, 95% or 100% as compared to a gene not targeted by the designed RNA molecules of the invention.

The consequences of silencing can be confirmed by examination of the outward properties of a plant cell or whole plant or other organism (e.g. pest) that take up the designed RNA from the plant or by biochemical techniques (as further discussed herein).

It will be appreciated that the designed RNA molecule of some embodiments of the invention can have some off-target specificity effect's provided that it does not affect an agriculturally valuable trait (e.g., biomass, yield, growth, etc. of the plant).

The specific binding of an RNA molecule (e.g. silencing molecule) with a target RNA can be determined by computational algorithms (such as BLAST) and verified by methods including e.g. Northern blot, In Situ hybridization, QuantiGene Plex Assay etc.

According to one embodiment, if the RNA molecule is or processed into a siRNA., the complementarily is in the range of 90-100% (e.g. 100%) to its target sequence.

According to one embodiment, if the RNA molecule is or processed into a miRNA or piRNA the complementarity is in the range of 33-100% to its target sequence.

According to one embodiment, if the RNA molecule is a miRNA, the seed sequence complementarity (i.e. nucleotides 2-8 from the 5′) is in the range of 85-100% (e.g. 100%) to its target sequence.

According to one embodiment, the complementarity to the target sequence is at least about 33% of the processed small RNA form (e.g. 33% of the 21-28 nt). Thus, for example, if the RNA molecule is a miRNA, 33% of the mature miRNA sequence (e.g. 21 nt) comprises seed complementation (e.g. 7 nt out of the 21 nt).

According to one embodiment, the complementarity to the target sequence is at least about 45% of the processed small RNA form (e.g. 45% of the 21-28 nt). Thus, for example, if the RNA molecule is a miRNA, 45% of the mature miRNA sequence (e.g. 21 nt) comprises seed complementation (e.g. 9-10 nt out of the 21 nt).

According to one embodiment, the RNA molecule or plant RNA (i.e, prior to modification) is typically selected as one having about 10%, 20%, 30%, 33%, 40%, 50%, 60%, 70%, 80%, 85%, 90%, 95%, 96%, 97%, 98% or up to 99% complementarity towards the sequence of the plant RNA or pest RNA, respectively.

According to a specific embodiment, the RNA molecule or plant RNA (i.e. prior to modification) is typically selected as one having no more than 99% complementarity towards the sequence of the plant RNA or pest RNA, respectively.

According to a specific embodiment, the RNA molecule or plant RNA (i.e. prior to modification) is typically selected as one having no more than 98% complementarity towards the sequence of the plant RNA or pest RNA, respectively.

According to a specific embodiment, the RNA molecule or plant RNA (i.e. prior to modification) is typically selected as one having no more than 97% complementarity towards the sequence of the plant RNA or pest RNA, respectively.

According to a specific embodiment, the RNA molecule or plant RNA (i.e. prior to modification) is typically selected as one having no more than 96% complementarity towards the sequence of the plant RNA or pest RNA, respectively.

According to a specific embodiment, the RNA molecule or plant RNA (i.e. prior to modification) is typically selected as one having no more than 95% complementarity towards the sequence of the plant RNA or pest RNA, respectively.

According to a specific embodiment, the RNA molecule or plant RNA (i.e. prior to modification) is typically selected as one having no more than 94% complementarity towards the sequence of the plant RNA or pest RNA, respectively.

According to a specific embodiment, the RNA molecule or plant RNA (i.e. prior to modification) is typically selected as one having no more than 93% complementarity towards the sequence of the plant RNA or pest RNA, respectively.

According to a specific embodiment, the RNA molecule or plant RNA (i.e. prior to modification) is typically selected as one having no more than 92% complementarity towards the sequence of the plant RNA or pest RNA, respectively.

According to a specific embodiment, the RNA molecule or plant RNA (i.e. prior to modification) is typically selected as one having no more than 91% complementarity towards the sequence of the plant RNA or pest RNA, respectively.

According to a specific embodiment, the RNA molecule or plant RNA (i.e. prior to modification) is typically selected as one having no more than 90% complementarity towards the sequence of the plant RNA or pest RNA, respectively.

According to a specific embodiment, the RNA molecule or plant RNA (i.e. prior to modification) is typically selected as one having no more than 85% complementarity towards the sequence of the plant RNA or pest RNA, respectively.

According to a specific embodiment, the RNA molecule or plant RNA (i.e. prior to modification) is typically selected as one having no more than 50% complementarity towards the sequence of the plant RNA or pest RNA, respectively.

According to a specific embodiment, the RNA molecule or plant RNA (i.e. prior to modification) is typically selected as one having no more than 33% complementarity towards the sequence of the plant RNA or pest RNA, respectively.

According to one embodiment, the RNA molecule (e.g. RNA silencing molecule) or plant RNA is designed so as to comprise at least about 33%, 40%, 45%, 50%, 55%, 60%, 70%, 80%, 85%, 90%, 91%, 92%, 93%, 94%, 95%, 96%, 97%, 98%, 99% or even 100% complementarity towards the sequence of the plant RNA or pest RNA, respectively.

According to a specific embodiment, the RNA molecule (e.g. RNA silencing molecule) or plant RNA is designed so as to comprise a minimum of 33% complementarity towards the plant RNA or pest RNA, respectively (e.g. 85-100% seed match).

According to a specific embodiment, the RNA molecule (e.g. RNA silencing molecule) or plant RNA is designed so as to comprise a minimum of 40% complementarity towards the plant RNA or pest RNA, respectively.

According to a specific embodiment, the RNA molecule (e.g. RNA silencing molecule) or plant RNA is designed so as to comprise a minimum of 45% complementarity towards the plant RNA or pest RNA, respectively.

According to a specific embodiment, the RNA molecule (e.g. RNA silencing molecule) or plant RNA is designed so as to comprise a minimum of 50% complementarity towards the plant RNA or pest RNA, respectively.

According to a specific embodiment, the RNA molecule (e.g. RNA silencing molecule) or plant RNA is designed so as to comprise a minimum of 55% complementarity towards the plant RNA or pest RNA, respectively.

According to a specific embodiment, the RNA molecule (e.g. RNA silencing molecule) or plant RNA is designed so as to comprise a minimum of 60% complementarity towards the plant RNA or pest RNA, respectively.

According to a specific embodiment, the RNA molecule (e.g. RNA silencing molecule) or plant RNA is designed so as to comprise a minimum of 70% complementarity towards the plant RNA or pest RNA, respectively.

According to a specific embodiment, the RNA molecule (e.g, RNA silencing molecule) or plant RNA is designed so as to comprise a minimum of 80% complementarity towards the plant RNA or pest RNA, respectively.

According to a specific embodiment, the RNA molecule (e.g. RNA silencing molecule) or plant RNA is designed so as to comprise a minimum of 85% complementarity towards the plant RNA or pest RNA, respectively.

According to a specific embodiment, the RNA molecule (e.g. RNA silencing molecule) or plant RNA is designed so as to comprise a minimum of 90% complementarity towards the plant RNA or pest RNA, respectively.

According to a specific embodiment, the RNA molecule (e.g, RNA silencing molecule) or plant RNA is designed so as to comprise a minimum of 91% complementarity towards the plant RNA or pest RNA, respectively.

According to a specific embodiment, the RNA molecule (e.g. RNA silencing molecule) or plant RNA is designed so as to comprise a minimum of 92% complementarity towards the plant RNA or pest RNA, respectively.

According to a specific embodiment, the RNA molecule (e.g. RNA silencing molecule) or plant RNA is designed so as to comprise a minimum of 93% complementarity towards the plant RNA or pest RNA, respectively.

According to a specific embodiment, the RNA molecule (e.g, RNA silencing molecule) or plant RNA is designed so as to comprise a minimum of 94% complementarity towards the plant RNA or pest RNA, respectively.

According to a specific embodiment, the RNA molecule (e.g. RNA silencing molecule) or plant RNA is designed so as to comprise a minimum of 95% complementarity towards the plant RNA or pest RNA, respectively.

According to a specific embodiment, the RNA molecule (e.g. RNA silencing molecule) or plant RNA is designed so as to comprise a minimum of 96% complementarity towards the plant RNA or pest RNA, respectively.

According to a specific embodiment, the RNA molecule (e.g. RNA silencing molecule) or plant RNA is designed so as to comprise a minimum of 97% complementarity towards the plant RNA or pest RNA, respectively.

According to a specific embodiment, the RNA molecule (e.g. RNA silencing molecule) or plant RNA is designed so as to comprise a minimum of 98% complementarity towards the plant RNA or pest RNA, respectively.

According to a specific embodiment, the RNA molecule (e.g, RNA silencing molecule) or plant RNA is designed so as to comprise a minimum of 99% complementarity towards the plant RNA or pest RNA, respectively.

According to a specific embodiment, the RNA molecule (e.g. RNA silencing molecule) or plant RNA is designed so as to comprise 100% complementarity towards the plant RNA or pest RNA, respectively.

According to a specific embodiment, the anti-sense strand of the RNA molecule or plant RNA the product synthesized by RdRp) is designed so as to comprise at least about 33%, 40%, 45%, 50%, 55%, 60%, 70%, 80%, 85%, 90%, 91%, 92%, 93%, 94%, 95%, 96%, 97%, 98%, 99% or even 100% complementarity towards the sequence of the pest RNA.

According to a specific embodiment, the anti-sense strand of the RNA molecule or plant RNA (e.g, the product synthesized by RdRp) is designed so as to comprise a minimum of 33% complementarity towards the sequence of the pest RNA. (e.g. 85-100% seed match).

According to a specific embodiment, the anti-sense strand of the RNA molecule or plant

RNA (e.g. the product synthesized by RdRp) is designed so as to comprise a minimum of 40% complementarity towards the sequence of the pest RNA.

According to a specific embodiment, the anti-sense strand of the RNA molecule or plant RNA (e.g. the product synthesized by RdRp) is designed so as to comprise a minimum of 45% complementarily towards the sequence of the pest RNA.

According to a specific embodiment, the anti-sense strand of the RNA molecule or plant RNA (e.g, the product synthesized by RdRp) is designed so as to comprise a minimum of 50% complementarity towards the sequence of the pest RNA.

According to a specific embodiment, the anti-sense strand of the RNA molecule or plant RNA (e.g. the product synthesized by RdRp) is designed so as to comprise a minimum of 55% complementarity towards the sequence of the pest RNA.

According to a specific embodiment, the anti-sense strand of the RNA molecule or plant RNA (e.g. the product synthesized by RdRp) is designed so as to comprise a minimum of 60% complementarily towards the sequence of the pest RNA.

According to a specific embodiment, the anti-sense strand of the RNA molecule or plant RNA (e.g. the product synthesized by RdRp) is designed so as to comprise a minimum of 70% complementarity towards the sequence of the pest RNA.

According to a specific embodiment, the anti-sense strand of the RNA molecule or plant RNA (e.g. the product synthesized by RdRp) is designed so as to comprise a minimum of 80% complementarity towards the sequence of the pest RNA.

According to a specific embodiment, the anti-sense strand of the RNA molecule or plant RNA (e.g. the product synthesized by RdRp) is designed so as to comprise a minimum of 85% complementarily towards the sequence of the pest RNA.

According to a specific embodiment, the anti-sense strand of the RNA molecule or plant RNA (e.g. the product synthesized by RdRp) is designed so as to comprise a minimum of 90% complementarity towards the sequence of the pest RNA.

According to a specific embodiment, the anti-sense strand of the RNA molecule or plant RNA (e.g. the product synthesized by RdRp) is designed so as to comprise a minimum of 91% complementarity towards the sequence of the pest RNA.

According to a specific embodiment, the anti-sense strand of the RNA molecule or plant RNA (e.g. the product synthesized by RdRp) is designed so as to comprise a minimum of 92% complementarily towards the sequence of the pest RNA.

According to a specific embodiment, the anti-sense strand of the RNA molecule or plant RNA (e.g. the product synthesized by RdRp) is designed so as to comprise a minimum of 93% complementarity towards the sequence of the pest RNA.

According to a specific embodiment, the anti-sense strand of the RNA molecule or plant RNA (e.g. the product synthesized by RdRp) is designed so as to comprise a minimum of 94% complementarity towards the sequence of the pest RNA.

According to a specific embodiment, the anti-sense strand of the RNA molecule or plant

RNA (e.g. the product synthesized by RdRp) is designed so as to comprise a minimum of 95% complementarily towards the sequence of the pest RNA.

According to a specific embodiment, the anti-sense strand of the RNA molecule or plant RNA (e.g. the product synthesized by RdRp) is designed so as to comprise a minimum of 96% complementarity towards the sequence of the pest RNA.

According to a specific embodiment, the anti-sense strand of the RNA molecule or plant RNA (e.g. the product synthesized by RdRp) is designed so as to comprise a minimum of 97% complementarity towards the sequence of the pest RNA.

According to a specific embodiment, the anti-sense strand of the RNA molecule or plant RNA (e.g. the product synthesized by RdRp) is designed so as to comprise a minimum of 98% complementarity towards the sequence of the pest RNA.

According to a specific embodiment, the anti-sense strand of the RNA molecule or plant RNA (e.g. the product synthesized by RdRp) is designed so as to comprise a minimum of 99% complementarity towards the sequence of the pest RNA.

According to a specific embodiment, the anti-sense strand of the RNA molecule or plant RNA (e.g. the product synthesized by RdRp) is designed so as to comprise 100% complementarity towards the sequence of the pest RNA.

In order to induce silencing activity and/or specificity of a RNA moleculeor a plant RNA or redirect a silencing activity and/or specificity of a RNA molecule or a plant RNA (e.g. RNA silencing molecule) towards a plant RNA or pest RNA, the gene encoding a RNA molecule or the plant RNA (e.g. RNA silencing molecule) is modified using a DNA editing agent.

Following is a description of various non-limiting examples of methods and DNA editing agents used to introduce nucleic acid alterations to a gene and agents for implementing same that can be used according to specific embodiments of the present disclosure.

Genome Editing using engineered endonucleases—this approach refers to a reverse genetics method using artificially engineered or modified naturally occurring nucleases to typically cut and create specific double-stranded breaks (DSB) at a desired location(s) in the genome, which are then repaired by cellular endogenous processes such as, homologous recombination (HR) or non-homologous end-joining (NHEJ). NHEJ directly joins the DNA ends in a double-stranded break (DSB) with or without minimal ends trimming, while HR utilizes a homologous donor sequence as a template (i.e. the sister chromatid formed during S-phase) for regenerating/copying the missing DNA sequence at the break site. In order to introduce specific nucleotide modifications to the genomic DNA, a donor DNA repair template containing the desired sequence must be present during HR (exogenously provided single stranded or double stranded DNA).

Genome editing cannot be performed using traditional restriction endonucleases since most restriction enzymes recognize a few base pairs on the DNA as their target and these sequences often will be found in many locations across the genome resulting in multiple cuts which are not limited to a desired location. To overcome this challenge and create site-specific single- or double-stranded breaks (DSBs), several distinct classes of nucleases have been discovered and bioengineered to date. These include the meganucleases, Zinc finger nucleases (ZFNs), transcription-activator like effector nucleases (TALENs) and CRISPR/Cas9 system.

Meganucleases—Meganucleases (also known as homing endonucleases) are commonly grouped into at least five four families: the LAGLIDADG family, the GIY-YIG family, the His-Cys box family and the HNH family and PD-(D/E)xK, which are related to EDxHD enzymes and are considered by some as a separate family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location.

This can be exploited to make site-specific double-stranded breaks (DSBs) in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence.

Alternatively, DNA interacting amino acids of the meganuclease can be altered to design sequence specific meganucleases e.g., U.S. Pat. No. 8,021,867). Meganucleases can be designed using the methods described in Certo, MT et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (DSBs) (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break (DSB).

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break (DSB). Repair of these double-stranded breaks (DSBs) through the non-homologous end-joining (NHEJ) pathway often results in small deletions or small sequence insertions (Indels). Since each repair made by NHEJ is unique, the use of a single nuclease pair can produce an allelic series with a range of different insertions or deletions at the target site.

In general NHEJ is relatively accurate (about 85% of DSBs in human cells are repaired by NHEJ within about 30 min from detection) in gene editing erroneous NHEJ is relied upon as when the repair is accurate the nuclease will keep cutting until the repair product is mutagenic and the recognition/cut site/PAM motif is gone/mutated or that the transiently introduced nuclease is no longer present.

The deletions typically range anywhere from a few base pairs to a few hundred base pairs in length, but larger deletions have been successfully generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break (DSB) can be repaired via homologous recombination (HR) to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and. TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5)460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

T-GEE system (TargetGene's Genome Editing Engine)—A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence. Nucleoprotein composition comprises (a) polynucleotide molecule encoding a chimeric polypeptide and comprising (i) a functional domain capable of modifying the target site, and (ii) a linking domain that is capable of interacting with a specificity conferring nucleic acid, and (b) specificity conferring nucleic acid (SCNA) comprising (i) a nucleotide sequence complementary to a region of the target nucleic acid flanking the target site, and (ii) a recognition region capable of specifically attaching to the linking domain of the polypeptide. The composition enables modifying a predetermined nucleic acid sequence target precisely, reliably and cost-effectively with high specificity and binding capabilities of molecular complex to the target nucleic acid through base-pairing of specificity-conferring nucleic acid and a target nucleic acid. The composition is less genotoxic, modular in their assembly, utilize single platform without customization, practical for independent use outside of specialized core-facilities, and has shorter development time frame and reduced costs.

CRISPR-Cas system and all its variants (also referred to herein as “CRISPR”)—Many bacteria and archea contain endogenous RNA-based adaptive immune systems that can degrade nucleic acids of invading phages and plasmids. These systems consist of clustered regularly interspaced short palindromic repeat (CRISPR) nucleotide sequences that produce RNA components and CRISPR associated (Cas) genes that encode protein components. The CRISPR RNAs (crRNAs) contain short stretches of homology to the DNA of specific viruses and plasmids and act as guides to direct Cas nucleases to degrade the complementary nucleic acids of the corresponding pathogen. Studies of the type II CIUSPR/Cas system of Streptococcus pyogenes have shown that three components form a RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821).

It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded breaks (DSBs) in a variety of different species (Cho et al, 2013; Cong et al., 2013; DiCarlo et al., 2013; Hwang et al., 2013a,b; Jinek et al, 2013; Mali et al., 2013).

The CRISPR/Cas system for genome editing contains two distinct components: a sgRNA and an endonuclease e.g. Cas9.

The gRNA (also referred to herein as short guide RNA (sgRNA)) is typically a 20-nucleotide sequence encoding a combination of the target homologous sequence (crRNA) and the endogenous bacterial RNA that links the crRNA to the Cas9 nuclease (tracrRNA) in a single chimeric transcript. The sgRNA/Cas9 complex is recruited to the target sequence by the base-pairing between the sgRNA sequence and the complement genomic DNA. For successful binding of Cas9, the genomic target sequence must also contain the correct Protospacer Adjacent Motif (PAM) sequence immediately following the target sequence. The binding of the sgRNA/Cas9 complex localizes the Cas9 to the genomic target sequence so that the Cas9 can cut both strands of the DNA causing a double-strand break (DSB). Just as with ZFNs and TALENs, the double-stranded breaks (DSBs) produced by CRISPR/Cas can undergo homologous recombination or NHEJ and are susceptible to specific sequence modification during DNA repair.

The Cas9 nuclease has two functional domains: RuvC and HNH, each cutting a different DNA strand. When both of these domains are active, the Cas9 causes double strand breaks (DSBs) in the genomic DNA.

A significant advantage of CRISPR/Cas is that the high efficiency of this system is coupled with the ability to easily create synthetic gRNAs. This creates a system that can be readily modified to target modifications at different genomic sites and/or to target different modifications at the same site. Additionally, protocols have been established which enable simultaneous targeting of multiple genes. The majority of cells carrying the mutation present biallelic mutations in the targeted genes.

However, apparent flexibility in the base-pairing interactions between the sgRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is mostly repaired by single strand break repair mechanism involving proteins such as but not only, PARP (sensor) and XRCC1/LIG III complex (ligation). If a single strand break (SSB) is generated by topoisomerase I poisons or by drugs that trap PARP1 on naturally occurring SSBs then these could persist and when the cell enters into S-phase and the replication fork encounter such SSBs they will become single ended DSBs which can only be repaired by HR. However, two proximal, opposite strand nicks introduced by a Cas9 nickase are treated as a double-strand break, in what is often referred to as a ‘double nick’ CRISPR system. A double-nick, which is basically non-parallel DSB, can be repaired like other DSBs by HR or NHEJ depending on the desired effect on the gene target and the presence of a donor sequence and the cell cycle stage (HR is of much lower abundance and can only occur in S and G2 stages of the cell cycle). Thus, if specificity and reduced off-target effects are crucial, using the Cas9 nickase to create a double-nick by designing two sgRNAs with target sequences in close proximity and on opposite strands of the genomic DNA would decrease off-target effect as either sgRNA alone will result in nicks that are not likely to change the genomic DNA, even though these events are not impossible.

Modified versions of the Cas9 enzyme containing two inactive catalytic domains (dead Cas9, or dCas9) have no nuclease activity while still able to bind to DNA based on sgRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

Additional variants of Cas9 which may be used by some embodiments of the invention include, but are not limited to, CasX and Cpf1. CasX enzymes comprise a distinct family of RNA-guided genome editors which are smaller in size compared to Cas9 and are found in bacteria (which is typically not found in humans), hence, are less likely to provoke the immune system/response in a human. Also, CasX utilizes a different PAM motif compared to Cas9 and therefore can be used to target sequences in which Cas9 PAM motifs are not found [see Liu J J et al., Nature. (2019) 566(7743):218-223.].Cpf1, also referred to as Cas12a, is especially advantageous for editing AT rich regions in which Cas9 PAMs (NGG) are much less abundant [see Li T et al., Biotechnol Adv. (2019) 37(0:21-27; Murugan K et al., Mol Cell (2017) 68(1):15-25].

According to another embodiment, the CRISPR system may be fused with various effector domains, such as DNA cleavage domains. The DNA cleavage domain can be obtained from any endonuclease or exonuclease. Non-limiting examples of endonucleases from which a DNA cleavage domain can be derived include, but are not limited to, restriction endonucleases and homing endonucleases (see, for example, New England Biolabs Catalog or Belfort et al. (1997) Nucleic Acids Res.). In exemplary embodiments, the cleavage domain of the CRISPR system is a Fokl endonuclease domain or a modified Fokl endonuclease domain. In addition, the use of Homing Endonucleases (HE) is another alternative. HEs are small proteins (<300 amino acids) found in bacteria, archaea, and in unicellular eukaryotes. A distinguishing characteristic of HEs is that they recognize relatively long sequences (14-40 bp) compared to other site-specific endonucleases such as restriction enzymes (4-8 bp). HEs have been historically categorized by small conserved amino acid motifs. At least five such families have been identified: LAGLIDADG; GIY-YIG; HNH; His-Cys Box and PD-(D/E)xK, which are related to EDxHD enzymes and are considered by some as a separate family. At a structural level, the HNH and His-Cys Box share a common fold (designated ββα-metal) as do the PD-(D/E)xK and EDxHD enzymes. The catalytic and DNA recognition strategies for each of the families vary and lend themselves to different degrees to engineering for a variety of applications. See e.g. Methods Mol Biol (2014) 1123:1-26. Exemplary Homing Endonucleases which may be used according to some embodiments of the invention include, without being limited to, I-CreI, I-TevI, I-HmuI, I-PpoI and I-Ssp68031.

Modified versions of CRISPR, e.g. dead CRISPR (dCRISPR-endonuclease), may also be utilized for CRISPR transcription inhibition (CRISPRi) or CRISPR transcription activation (CRISPRa) see e.g. Kampmann M., ACS Chem Biol. (2018) 13(2):406-416; La Russa M F and Qi L S., Mol Cell Biol (2015) 35(22):3800-9].

Other versions of CRISPR which may be used according to some embodiments of the invention include genome editing using components from CRISPR systems together with other enzymes to directly install point mutations into cellular DNA or RNA.

Thus, according to one embodiment, the editing agent is DNA or RNA editing agent.

According to one embodiment, the DNA or RNA editing agent elicits base editing.

The term “base editing” as used herein refers to installing point mutations into cellular DNA or RNA without making double-stranded or single-stranded DNA breaks.

In base editing, DNA base editors typically comprise fusions between a catalytically impaired Cas nuclease and a base modification enzyme that operates on single-stranded DNA (ssDNA). Upon binding to its target DNA locus, base pairing between the gRNA and the target DNA strand leads to displacement of a small segment of single-stranded DNA in an ‘R loop’. DNA bases within this ssDNA bubble are modified by the base-editing enzyme (e.g. deaminase enzyme). To improve efficiency in eukaryotic cells, the catalytically disabled nuclease also generates a nick in the non-edited DNA strand, inducing cells to repair the non-edited strand using the edited strand as a template.

Two classes of DNA base editor have been described: cytosine base editors (CBEs) convert a C-G base pair into a T-A base pair, and adenine base editors (ABEs) convert an A-T base pair into a G-C base pair. Collectively, CBEs and ABEs can mediate all four possible transition mutations (C to T, A to G, T to C and G to A). Similarly in RNA, targeted adenosine conversion to inosine utilizes both antisense and Cas13-guided RNA—targeting methods.

According to one embodiment, the DNA or RNA editing agent comprises a catalytically inactive endonuclease (e.g. CRISPR-dCas).

According to one embodiment, the catalytically inactive endonuclease is an inactive Cas9 (e.g. dCas9).

According to one embodiment, the catalytically inactive endonuclease is an inactive Cas13 (e.g. dCas13).

According to one embodiment, the DNA or RNA editing agent comprises an enzyme which is capable of epigenetic editing (i.e. providing chemical changes to the DNA, the RNA or the histone proteins).

Exemplary enzymes include, but are not limited to, DNA methyltransferases, methylases, acetyltransferases. More specifically, exemplary enzymes include e.g. DNA (cytosine-5)-methyltransferase 3A (DNMT3a), Histone acetyltransferase p300, Ten-eleven translocation methylcytosine dioxygenase 1 (TET 1), Lysine (K)-specific demethylase 1A (LSD1) and Calcium and integrin binding protein 1 (CIB1).

In addition to the catalytically disabled nuclease, the DNA or RNA editing agents of the invention may also comprise a nucleobase deaminase enzyme and/or a DNA glycosylase inhibitor.

According to a specific embodiment, the DNA or RNA editing agents comprise BE1 (APOBEC1-XTEN-dCas9), BE2 (APOBEC1-XTEN-dCas9-UGI) or BE3 (APOBEC-XTEN-dCas9(A840H)-UGI), along with sgRNA. APOBEC1 is a deaminase full length or catalytically active fragment, XTEN is a protein linker, UGI is uracil DNA glycosylase inhibitor to prevent the subsequent U:G mismatch from being repaired back to a C:G base pair and dCas9 (A840H) is a nickase in which the dCas9 was reverted to restore the catalytic activity of the HNH domain which nicks only the non-edited strand, simulating newly synthesized DNA and leading to the desired U:A product.

Additional enzymes which can be used for base editing according to some embodiments of the invention are specified in Rees and Liu, Nature Reviews Genetics (2018) 19:770-788, incorporated herein by reference in its entirety.

There are a number of publicly available tools available to help choose and/or design target sequences as well as lists of bioinformatically determined unique sgRNAs for different genes in different species such as, but not limited to, the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both sgRNA and a Cas endonuclease (e.g. Cas9) should be expressed or present (e.g., as a ribonucleoprotein complex) in a target cell. The insertion vector can contain both cassettes on a single plasmid or the cassettes are expressed from two separate plasmids. CRISPR plasmids are commercially available such as the px330 plasmid from Addgene (75 Sidney St, Suite 550A. Cambridge, Mass. 02139). Use of clustered regularly interspaced short palindromic repeats (CRISPR)-associated (Cas)-guide RNA technology and a Cas endonuclease for modifying plant genomes are also at least disclosed by Svitashev et al., 2015, Plant Physiology, 169 (2): 931-945; Kumar and. Jain, 2015, J Exp Bot 66: 47-57; and in U.S. Patent Application Publication No. 20150082478, which is specifically incorporated herein by reference in its entirety. Cas endonucleases that can be used to effect DNA editing with sgRNA include, but are not limited to, Cas9, Cpf1 (Zetsche et al., 2015, Cell. 163(3):759-71), C2c1, C2c2, and C2c3 (Shmakov et al., Mol Cell. 2015 Nov. 5;60(3):385-97).

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, introduced into the cells, and positive selection is performed to isolate homologous recombination mediated events. The DNA carrying the homologous sequence can be provided as a plasmid, single or double stranded oligo. These homologous recombinants contain a local duplication that is separated by intervening vector sequence, including the selection cassette. In the second step, targeted clones are subjected to negative selection to identify cells that have lost the selection cassette via intra-chromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to he introduced. After the system components have been introduced to the cell and positive selection applied, HR mediated events could be identified. Next, a second targeting vector that contains a region of homology with the desired mutation is introduced into targeted clones, and negative selection is applied to remove the selection cassette and introduce the mutation. The final allele contains the desired mutation while eliminating unwanted exogenous sequences.

According to a specific embodiment, the DNA editing agent comprises a DNA targeting module (e.g., sgRNA).

According to a specific embodiment, the DNA editing agent does not comprise an endonuclease.

According to a specific embodiment, the DNA editing agent comprises a nuclease (e.g. an endonuclease) and a DNA targeting module (e.g., sgRNA).

According to a specific embodiment, the DNA editing agent is CRISPR/Cas, e.g. sgRNA and Cas9.

According to a specific embodiment, the DNA editing agent is TALEN.

According to a specific embodiment, the DNA editing agent is ZFN.

According to a specific embodiment, the DNA editing agent is meganuclease.

According to a specific embodiment, the DNA editing agent comprises a CRISPR. endonuclease and an sgRNA directed at cutting the plant gene.

According to a specific embodiment, an oligonucleotide serving as a template for Homology Dependent Recombination (HDR) is introduced to the cell together with the DNA editing agent, wherein the oligonucleotide comprises a sequence of the plant gene with nucleotide changes which enable modifying the nucleic acid sequence of the plant gene so as to impart a silencing specificity towards the pest gene.

According to one embodiment, the DNA editing agent is linked to a reporter for monitoring expression in a plant cell.

According to one embodiment, the reporter is a fluorescent reporter protein.

The term “a fluorescent protein” refers to a polypeptide that emits fluorescence and is typically detectable by flow cytometry, microscopy or any fluorescent imaging system, therefore can be used as a basis for selection of cells expressing such a protein.

Examples of fluorescent proteins that can be used as reporters are, without being limited to, the Green Fluorescent Protein (GFP), the Blue Fluorescent Protein (BFP) and the red fluorescent proteins (e.g. dsRed, mCherry, RFP). A non-limiting list of fluorescent or other reporters includes proteins detectable by luminescence (e.g. luciferase) or colorimetric assay (e.g. GUS). According to a specific embodiment, the fluorescent reporter is a red fluorescent protein (e.g. dsRed, mCherry, RFP) or GFP.

A review of new classes of fluorescent proteins and applications can be found in Trends in Biochemical Sciences [Rodriguez, Erik A.; Campbell, Robert E; Lin, John Y.; Lin, Michael Z.; Miyawaki, Atsushi; Palmer, Amy E.; Shu, Xiaokun; Zhang, Jim; Tsien, Roger Y. “The Growing and Glowing Toolbox of Fluorescent and Photoactive Proteins”. Trends in Biochemical Sciences. doi:10.10.16/j.tibs.2016.09.010].

According to another embodiment, the reporter is an endogenous gene of a plant. An exemplary reporter is the phytoene desaturase gene (PDS3) which encodes one of the important enzymes in the carotenoid biosynthesis pathway. Its silencing produces an albino/bleached phenotype. Accordingly, plants with reduced expression of PDS3 exhibit reduced chlorophyll levels, up to complete albino and dwarfism. Additional genes which can be used in accordance with the present teachings include, but are not limited to, genes which take part in crop protection.

According to another embodiment, the reporter is an antibiotic selection marker. Examples of antibiotic selection markers that can be used as reporters are, without being limited to, neomycin phosphotransferase II (nptII) and hygromycin phosphotransferase (hpt). Additional marker genes which can be used in accordance with the present teachings include, but are not limited to, gentamycin acetyltransferase (accC3) resistance and bleomycin and phleomycin resistance genes.

It will be appreciated that the enzyme NPTII inactivates by phosphorylation a number of aminoglycoside antibiotics such as kanamycin, neomycin, geneticin (or G418) and paromomycin. Of these, kanamycin, neomycin and paromomycin are used in a diverse range of plant species.

According to another embodiment, the reporter is a toxic selection marker. An exemplary toxic selection marker that can be used as a reporter is, without being limited to, allyl alcohol selection using the Alcohol dehydrogenase (ADH1) gene. ADH1, comprising a group of dehydrogenase enzymes which catalyse the interconversion between alcohols and aldehydes or ketones with the concomitant reduction of NAD+ or NADP+, breaks down alcoholic toxic substances within tissues. Plants harbouring reduced ADH1 expression exhibit increase tolerance to allyl alcohol. Accordingly, plants with reduced ADH1 are resistant to the toxic effect of allyl alcohol.

Regardless of the DNA editing agent used, the method of the invention is employed such that the gene encoding the RNA molecule or the plant gene (e.g. RNA silencing molecule) is modified by at least one of a deletion, an insertion or a point mutation.

According to one embodiment, the modification is in a structured region of a non-coding RNA molecule (e.g. RNA silencing molecule).

According to one embodiment, the modification is in a stem region of a non-coding RNA molecule (e.g. RNA silencing molecule).

According to one embodiment, the modification is in a loop region of a non-coding RNA molecule (e.g. RNA silencing molecule).

According to one embodiment, the modification is in a stem region and a loop region of a non-coding RNA molecule (e.g. RNA silencing molecule).

According to one embodiment, the modification is in a non-structured region of a non-coding RNA molecule (e.g. RNA silencing molecule).

According to one embodiment, the modification is in a stem region and a loop region and in non-structured region of a non-coding RNA molecule (e.g. RNA silencing molecule).

According to a specific embodiment, the modification comprises a modification of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native plant RNA or native RNA molecule, e.g. RNA silencing molecule).

According to one embodiment, the modification comprises a modification of at most 1, 2, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native plant RNA or native RNA molecule, e.g. RNA silencing molecule).

According to one embodiment, the modification can be in a consecutive nucleic acid sequence (e.g. at least 5, 10, 20, 30, 40, 50, 100, 150, 200 bases)

According to one embodiment, the modification can be in a non-consecutive manner, e.g. throughout a 20, 50, 100, 150, 200, 500, 1000 nucleic acid sequence.

According to a specific embodiment, the modification comprises a modification of at most 200 nucleotides.

According to a specific embodiment, the modification comprises a modification of at most 150 nucleotides.

According to a specific embodiment, the modification comprises a modification of at most 100 nucleotides.

According to a specific embodiment, the modification comprises a modification of at most 50 nucleotides.

According to a specific embodiment, the modification comprises a modification of at most 25 nucleotides.

According to a specific embodiment, the modification comprises a modification of at most 20 nucleotides.

According to a specific embodiment, the modification comprises a modification of at most 15 nucleotides.

According to a specific embodiment, the modification comprises a modification of at most 10 nucleotides.

According to a specific embodiment, the modification comprises a modification of at most 5 nucleotides.

According to one embodiment, the modification depends on the structure of the RNA molecule (e.g. silencing molecule).

Accordingly, when the RNA molecule contains a non-essential structure (i.e. a secondary structure of a RNA silencing molecule which does not play a role in its proper biogenesis and/or function) or is purely dsRNA (i.e. the RNA silencing molecule having a perfect or almost perfect dsRNA), a few modifications (e.g. 20-30 nucleotides, e.g. 1-10 nucleotides, e.g. 5 nucleotides) are introduced in order to redirect the silence specificity of the RNA molecule.

According to another embodiment, when the RNA molecule has an essential structure (i.e. the proper biogenesis and/or activity of the RNA silencing molecule is dependent on its secondary structure), larger modifications (e.g. 10-200 nucleotides, e.g. 50-150 nucleotides, e.g., more than 30 nucleotides and not exceeding 200 nucleotides, 30-200 nucleotides, 35-200 nucleotides, 35-150 nucleotides, 35-100 nucleotides) are introduced in order to redirect the silence specificity of the RNA molecule.

According to one embodiment, the modification is such that the recognition/cut site/PAM motif of the RNA silencing molecule is modified to abolish the original PAM recognition site.

According to a specific embodiment, the modification is in at least 1, 2., 4, 5, 6, 7, 8, 9, 10 or more nucleic acids in a PAM motif.

According to one embodiment, the modification comprises an insertion.

According to a specific embodiment, the insertion comprises an insertion of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native plant RNA or native RNA molecule, e.g. RNA silencing molecule).

According to one embodiment, the insertion comprises an insertion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native plant RNA or native RNA molecule, e.g. RNA silencing molecule).

According to a specific embodiment, the insertion comprises an insertion of at most 200 nucleotides.

According to a specific embodiment, the insertion comprises an insertion of at most 150 nucleotides.

According to a specific embodiment, the insertion comprises an insertion of at most 100 nucleotides.

According to a specific embodiment, the insertion comprises an insertion of at most 50 nucleotides.

According to a specific embodiment, the insertion comprises an insertion of at most 25 nucleotides.

According to a specific embodiment, the insertion comprises an insertion of at most 20 nucleotides.

According to a specific embodiment, the insertion comprises an insertion of at most 15 nucleotides.

According to a specific embodiment, the insertion comprises an insertion of at most 10 nucleotides.

According to a specific embodiment, the insertion comprises an insertion of at most 5 nucleotides.

According to one embodiment, the modification comprises a deletion.

According to a specific embodiment, the deletion comprises a deletion of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native plant RNA or native RNA molecule, e.g. RNA silencing molecule).

According to one embodiment, the deletion comprises a deletion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native plant RNA or native RNA molecule, e.g. RNA silencing molecule).

According to a specific embodiment, the deletion comprises a deletion of at most 200 nucleotides.

According to a specific embodiment, the deletion comprises a deletion of at most 150 nucleotides.

According to a specific embodiment, the deletion comprises a deletion of at most 100 nucleotides.

According to a specific embodiment, the deletion comprises a deletion of at most 50 nucleotides.

According to a specific embodiment, the deletion comprises a deletion of at most 25 nucleotides.

According to a specific embodiment, the deletion comprises a deletion of at most 20 nucleotides.

According to a specific embodiment, the deletion comprises a deletion of at most 15 nucleotides.

According to a specific embodiment, the deletion comprises a deletion of at most 10 nucleotides.

According to a specific embodiment, the deletion comprises a deletion of at most 5 nucleotides.

According to one embodiment, the modification comprises a point mutation,

According to a specific embodiment, the point mutation comprises a point mutation of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native plant RNA or native RNA molecule, e.g, RNA silencing molecule),

According to one embodiment, the point mutation comprises a point mutation in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native plant RNA or native RNA molecule, e.g. RNA silencing molecule).

According to a specific embodiment, the point mutation comprises a point mutation in at most 200 nucleotides.

According to a specific embodiment, the point mutation comprises a point mutation in at most 150 nucleotides.

According to a specific embodiment, the point mutation comprises a point mutation in at most 100 nucleotides.

According to a specific embodiment, the point mutation comprises a point mutation in at most 50 nucleotides.

According to a specific embodiment, the point mutation comprises a point mutation in at most 25 nucleotides.

According to a specific embodiment, the point mutation comprises a point mutation in at most 20 nucleotides.

According to a specific embodiment, the point mutation comprises a point mutation in at most 15 nucleotides.

According to a specific embodiment, the point mutation comprises a point mutation in at most 10 nucleotides.

According to a specific embodiment, the point mutation comprises a point mutation in at most 5 nucleotides.

According to one embodiment, the modification comprises a combination of any of a deletion, an insertion and/or a point mutation.

According to one embodiment, the modification comprises nucleotide replacement (e.g. nucleotide swapping).

According to a specific embodiment, the swapping comprises swapping of about 10-250 nucleotides, about 10-200 nucleotides, about 10-150 nucleotides, about 10-100 nucleotides, about 10-50 nucleotides, about 1-50 nucleotides, about 1-10 nucleotides, about 50-150 nucleotides, about 50-100 nucleotides or about 100-200 nucleotides (as compared to the native plant RNA or native RNA molecule, e.g. RNA silencing molecule).

According to one embodiment, the nucleotide swap comprises a nucleotide replacement in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 110, 120, 130, 140, 150, 160, 170, 180, 190, 200 or at most 250 nucleotides (as compared to the native plant RNA or native RNA molecule, e.g. RNA silencing molecule).

According to a specific embodiment, the nucleotide swapping comprises a nucleotide replacement in at most 200 nucleotides.

According to a specific embodiment, the nucleotide swapping comprises a nucleotide replacement in at most 150 nucleotides.

According to a specific embodiment, the nucleotide swapping comprises a nucleotide replacement in at most 100 nucleotides.

According to a specific embodiment, the nucleotide swapping comprises a nucleotide replacement in at most 50 nucleotides.

According to a specific embodiment, the nucleotide swapping comprises a nucleotide replacement in at most 25 nucleotides.

According to a specific embodiment, the nucleotide swapping comprises a nucleotide replacement in at most 20 nucleotides.

According to a specific embodiment, the nucleotide swapping comprises a nucleotide replacement in at most 15 nucleotides.

According to a specific embodiment, the nucleotide swapping comprises a nucleotide replacement in at most 10 nucleotides.

According to a specific embodiment, the nucleotide swapping comprises a nucleotide replacement in at most 5 nucleotides.

According to one embodiment, the gene encoding the plant RNA or RNA molecule (e.g. RNA silencing molecule) is modified by swapping a sequence of an endogenous RNA silencing molecule (e.g. miRNA) with a RNA silencing sequence of choice (e.g. siRNA).

According to one embodiment, the guide strand of the RNA molecule (e.g. RNA silencing molecule such as miRNA precursors (pri/pre-miRNAs) or siRNA precursors (dsRNA)) is modified to preserve originality of structure and keep the same base pairing profile.

According to one embodiment, the passenger strand of the RNA molecule (e.g. RNA silencing molecule such as miRNA precursors (pri/pre-miRNAs) or siRNA precursors (dsRNA)) is modified to preserve originality of structure and keep the same base pairing profile.

As used herein, the term “originality of structure” refers to the secondary RNA structure (i.e. base pairing profile). Keeping the originality of structure is important for correct and efficient biogenesis/processing of the non-coding RNA (e.g. RNA silencing molecule such as siRNA or miRNA) that is structure—and not purely sequence-dependent.

According to one embodiment, the RNA (e.g. RNA silencing molecule) is modified in the guide strand (silencing strand) as to comprise about 50-100% complementarity to the target RNA (as discussed above) while the passenger strand is modified to preserve the original (unmodified) RNA (e.g. non-coding RNA) structure.

According to one embodiment, the RNA sequence (e.g. RNA silencing molecule) is modified such that the seed sequence (e.g. for miRNA nucleotides 2-8 from the 5′ terminal) is complimentary to the target sequence.

According to a specific embodiment, the RNA silencing molecule (i.e. RNAi molecule) is designed such that a sequence of the RNAi molecule is modified to preserve originality of structure and to be recognized by cellular RNAi processing and executing factors.

According to a specific embodiment, the RNA molecule e.g. non-coding RNA molecule (i.e. rRNA, tRNA, lncRNA, snoRNA, etc.) is designed such that a sequence of the RNAi molecule is modified to be recognized by cellular RNAi processing and executing factors.

It will be appreciated that additional mutations can be introduced by additional events of editing (i.e., concomitantly or sequentially).

The DNA editing agent of the invention may be introduced into plant cells using DNA delivery methods (e.g. by expression vectors) or using DNA-free methods.

According to one embodiment, the sgRNA (or any other DNA recognition module used, dependent on the I)NA editing system that is used) can be provided as RNA to the cell.

Thus, it will be appreciated that the present techniques relate to introducing the DNA editing agent using transient DNA or DNA-free methods such as RNA transfection (e.g. mRNA+sgRNA transfection), or Ribonucleoprotein (RNP) transfection (e.g. protein-RNA complex transfection, e.g. Cas9/sgRNA ribonucleoprotein (RNP) complex transfection).

For example, Cas9 can be introduced as a DNA expression plasmid, in vitro transcript (i.e. RNA), or as a recombinant protein bound to the RNA portion in a ribonucleoprotein particle (RNP). sgRNA., for example, can be delivered either as a DNA plasmid or as an in vitro transcript (i.e. RNA).

Any method known in the art for RNA or RNP transfection can be used in accordance with the present teachings, such as, but not limited to microinjection [as described by Cho et al., “Heritable gene knockout in Caenorhabditis elegans by direct injection of Cas9-sgRNA ribonucleoproteins,” Genetics (2013) 195:1177-1180, incorporated herein by reference], electroporation [as described by Kim et al., “Highly efficient RNA-guided genome editing in human cells via delivery of purified Cas9 ribonucleoproteins” Genome Res. (2014) 24:1012-1019, incorporated herein by reference], or lipid-mediated transfection e.g. using liposomes [as described by Zuris et al., “Cationic lipid-mediated delivery of proteins enables efficient protein-based genome editing in vitro and in vivo” Nat Bietechnol. (2014) doi: 10.1038/nbt.3081, incorporated herein by reference]. Additional methods of RNA transfection are described in U.S. Patent Application No. 20160289675, incorporated herein by reference in its entirety.

One advantage of RNA transfection methods of the invention is that RNA transfection is essentially transient and vector-free. A RNA transgene can be delivered to a cell and expressed therein, as a minimal expressing cassette without the need for any additional sequences (e.g. viral sequences).

According to one embodiment, the DNA editing agent of the invention is introduced into the plant cell using expression vectors.

The “expression vector” (also referred to herein as “a nucleic acid construct”, “vector” or “construct”) of some embodiments of the invention includes additional sequences which render this vector suitable for replication in prokaryotes, eukaryotes, or preferably both (e.g., shuttle vectors).

Constructs useful in the methods according to some embodiments of the invention may be constructed using recombinant DNA technology well known to persons skilled in the art. The nucleic acid sequences may be inserted into vectors, which may be commercially available, suitable for transforming into plants and suitable for transient expression of the gene of interest in the transformed cells. The genetic construct can be an expression vector wherein the nucleic acid sequence is operably linked to one or more regulatory sequences allowing expression in the plant cells.

According to one embodiment, in order to express a functional DNA editing agent, in cases where the cleaving module (nuclease) is not an integral part of the DNA recognition unit, the expression vector may encode the cleaving module as well as the DNA recognition unit (e.g. sgRNA in the case of CRISPR/Cas).

Alternatively, the cleaving module (nuclease) and the DNA recognition unit (e.g. sgRNA) may be cloned into separate expression vectors. In such a case, at least two different expression vectors must be transformed into the same plant cell.

Alternatively, when a nuclease is not utilized (i.e. not administered from an exogenous source to the cell), the DNA recognition unit (e.g. sgRNA) may be cloned and expressed using a single expression vector.

Typical expression vectors may also contain a transcription and translation initiation sequence, transcription and translation terminator and optionally a polyadenylation signal.

According to one embodiment, the DNA editing agent comprises a nucleic acid agent encoding at least one DNA recognition unit (e.g. sgRNA) operatively linked to a cis-acting regulatory element active in plant cells (e.g., promoter).

According to one embodiment, the nuclease (e.g. endonuclease) and the DNA recognition unit (e.g. sgRNA) are encoded from the same expression vector. Such a vector may comprise a single cis-acting regulatory element active in plant cells (e.g., promoter) for expression of both the nuclease and the DNA recognition unit. Alternatively, the nuclease and the DNA recognition unit may each be operably linked to a cis-acting regulatory element active in plant cells (e.g., promoter).

According to one embodiment, the nuclease (e.g. endonuclease) and the DNA recognition unit (e.g. sgRNA) are encoded from different expression vectors whereby each is operably linked to a cis-acting regulatory element active in plant cells (e.g., promoter).

As used herein the phrase “plant-expressible” or “active in plant cells” refers to a promoter sequence, including any additional regulatory elements added thereto or contained therein, that is at least capable of inducing, conferring, activating or enhancing expression in a plant cell, tissue or organ, preferably a monocotyledonous or dicotyledonous plant cell, tissue, or organ.

The plant promoter employed can be a constitutive promoter, a tissue specific promoter, an inducible promoter, a chimeric promoter or a developmentally regulated promoter.

Examples of preferred promoters useful for the methods of some embodiments of the invention are presented in Table I, II, III and IV.

TABLE I
Exemplary constitutive promoters for use in the performance of some embodiments of the invention

| Gene Source | Expression Pattern | Reference |
|---|---|---|
| Actin | constitutive | McElroy et al, Plant Cell, 2: 163-171, 1990 |
| CAMV 35S | constitutive | Odell et al, Nature, 313: 810-812, 1985 |
| CaMV 19S | constitutive | Nilsson et al., Physiol. Plant 100: 456-462, 1997 |
| GOS2 | constitutive | de Pater et al, Plant J Nov; 2(6): 837-44, 1992 |
| ubiquitin | constitutive | Christensen et al, Plant Mol. Biol. 18: 675-689, 1992 |
| Rice cyclophilin | constitutive | Bucholz et al, Plant Mol Biol. 25(5): 837-43, 1994 |
| Maize H3 histone | constitutive | Lepetit et al, Mol. Gen. Genet. 231: 276-285, 1992 |
| Actin 2 | constitutive | An et al, Plant J. 10(1); 107121, 1996 |
| CVMV (Cassava Vein Mosaic Virus | constitutive | Lawrenson et al, Gen Biol 16: 258, 2015 |
| U6 (AtU626; TaU6) | constitutive | Lawrenson et al, Gen Biol 16: 258, 2015 |

TABLE II
Exemplary seed-preferred promoters for use in the performance of some embodiments of the invention

| Gene Source | Expression Pattern | Reference |
|---|---|---|
| Seed specific genes | seed | Simon, et al., Plant Mol. Biol. 5. 191, 1985; |
| | | Scofield, et al., J. Biol. Chem. 262: 12202, |
| | | 1987; Baszczynski, et al., Plant Mol. Biol. |
| | | 14: 633, 1990. |
| Brazil Nut albumin | seed | Pearson' et al., Plant Mol. Biol. 18: |
| | | 235-245, 1992. |
| legumin | seed | Ellis, et al. Plant Mol. Biol. 10: 203-214, 1988 |
| Glutelin (rice) | seed | Takaiwa, et al., Mol. Gen. Genet. 208: |
| | | 15-22, 1986; Takaiwa, et al., FEBS Letts. 221: |
| | | 43-47, 1987 |
| Zein | seed | Matzke et al Plant Mol Biol, 143). 323-32 1990 |
| napA | seed | Stalberg, et al, Planta 199: 515-519, 1996 |
| wheat LMW and HMW | endosperm | Mol Gen Genet 216: 81-90, 1989; NAR 17: 461-2, |
| glutenin-1 | | |
| Wheat SPA | seed | Albanietal, Plant Cell, 9: 171-184, 1997 |
| wheat a, b and g gliadins | endosperm | EMBO3: 1409-15, 1984 |
| Barley ltrl promoter | endosperm | |
| barley B1, C, D hordein | endosperm | Theor Appl Gen 98: 1253-62, 1999; |
| | | Plant J 4: 343-55, 1993; Mol Gen Genet |
| | | 250: 750-60, 1996 |
| Barley DOF | endosperm | Mena et al, The Plant Journal, 116(1): 53-62, 1998 |
| Biz2 | endosperm | EP99106056.7 |
| Synthetic promoter | endosperm | Vicente-Carbajosa et al., Plant J. 13: 629-640, 1998 |
| rice prolamin NRP33 | endosperm | Wu et al, Plant Cell Physiology 39(8) 885-889, 1998 |
| rice -globulin Glb-1 | endosperm | Wu et al, Plant Cell Physiology 398) 885-889, 1998 |
| rice OSH1 | emryo | Sato et al, Proc. Nati. Acad. Sci. USA, 93: 8117-8122 |
| rice alpha-globulin | endosperm | Nakase et al. Plant Mol. Biol. 33: 513-S22, 1997 |
| REB/OHP-1 | | |
| rice ADP-glucose PP | endosperm | Trans Res 6: 157-68, 1997 |
| maize ESR gene family | endosperm | Plant J 12: 235-46, 1997 |
| sorgum gamma- kafirin | endosperm | PMB 32: 1029-35, 1996 |
| KNOX | emryo | Postma-Haarsma ef al, Plant Mol. Biol. 39: 257-71, 1999 |
| rice oleosin | Embryo and aleuton | Wu et at, J. Biochem., 123: 386, 1998 |
| sunflower oleosin | Seed (embryo and dry seed) | Cummins, et al., Plant Mol. Biol. 19: 873-876, 1992 |

TABLE III
Exemplary flower-specific promoters for
use in the performance of the invention

| Gene Source | Expression Pattern | Reference |
|---|---|---|
| AtPRP4 | flowers | www(dot)salus(dot) |
| | | medium(dot)edu/m |
| | | mg/tierney/html |
| chalene | flowers | Van der Meer, et al., |
| synthase (chsA) | | Plant Mol. Biol. |
| | | 15, 95-109, 1990. |
| LAT52 | anther | Twell et al Mol. Gen |
| | | Genet. 217: 240-245 (1989) |
| apetala- 3 | flowers | |

TABLE IV
Alternative rice promoters for use in the performance of the invention

| PRO # | Gene | Expression |
|---|---|---|
| PR00001 | Metallothionein Mte | transfer layer of embryo + calli |
| PR00005 | putative beta-amylase | transfer layer of embryo |
| PR00009 | Putative cellulose synthase | Weak in roots |
| PR00012 | lipase (putative) | |
| PR00014 | Transferase (putative) | |
| PR00016 | peptidyl prolyl cis-trans | |
| | isomerase (putative) | |
| PR00019 | unknown | |
| PR00020 | prp protein (putative) | |
| PR00029 | noduline (putative) | |
| PR00058 | Proteinase inhibitor Rgpi9 | seed |
| PR00061 | beta expansine EXPB9 | Weak in young flowers |
| PR00063 | Structural protein | young tissues + calli + |
| | | embryo |
| PR00069 | xylosidase (putative) | |
| PR00075 | Prolamine 10 Kda | strong in endosperm |
| PR00076 | allergen RA2 | strong in endosperm |
| PR00077 | prolamine RP7 | strong in endosperm |
| PR00078 | CBP80 | |
| PR00079 | starch branching enzyme I | |
| PR00080 | Metallothioneine-like ML2 | transfer layer of embryo + |
| | | calli |
| PR00081 | putative caffeoyl- CoA | shoot |
| | 3-0 methyltransferase | |
| PR00087 | prolamine RM9 | strong in endosperm |
| PR00090 | prolamine RP6 | strong in endosperm |
| PR00091 | prolamine RP5 | strong in endosperm |
| PR00092 | allergen RA5 | |
| PR00095 | putative | embryo |
| | methionine aminopeptidase | |
| PR00098 | ras-related GTP binding | |
| | protein | |
| PR00104 | beta expansine EXPB1 | |
| PR00105 | Glycine rich protein | |
| PR00108 | metallothionein like | |
| | protein (putative) | |
| PR00110 | RCc3 strong root | |
| PR00111 | uclacyanin 3-like protein | weak discrimination center/ |
| | | shoot meristem |
| PR00116 | 26S proteasome | very weak meristem |
| | regulatory particle | specific |
| | non-ATPase subunit 11 | |
| PR00117 | putative 40S ribosomal protein | weak in endosperm |
| PR00122 | chlorophyll a/lo-binding | very weak in shoot |
| | protein precursor (Cab27) | |
| PR00123 | putative | Strong leaves |
| | protochlorophyllide reductase | |
| PR00126 | metallothionein RiCMT | strong discrimination |
| | | center shoot meristem |
| PR00129 | GOS2 | Strong constitutive |
| PR00131 | GOS9 | |
| PR00133 | chitinase Cht-3 | very weak meristem |
| | | specific |
| PR00135 | alpha- globulin | Strong in endosperm |
| PR00136 | alanine aminotransferase | Weak in endosperm |
| PR00138 | Cyclin A2 | |
| PR00139 | Cyclin D2 | |
| PR00140 | Cyclin D3 | |
| PR00141 | Cyclophyllin 2 | Shoot and seed |
| PR00146 | sucrose synthase SS1 (barley) | medium constitutive |
| PR00147 | trypsin inhibitor ITR1 (barley) | weak in endosperm |
| PR00149 | ubiquitine 2 with intron | strong constitutive |
| PR00151 | WSI18 | Embryo and stress |
| PR00156 | HVA22 homologue (putative) | |
| PR00157 | EL2 | |
| PR00169 | aquaporine | medium constitutive in |
| | | young plants |
| PR00170 | High mobility group protein | Strong constitutive |
| PR00171 | reversibly glycosylated | weak constitutive |
| | protein RGP1 | |
| PR00173 | cytosolic MDH | shoot |
| PR00175 | RAB21 | Embryo and stress |
| PR00176 | CDPK7 | |
| PR00177 | Cdc2-1 | very weak in meristem |
| PR00197 | sucrose synthase 3 | |
| PRO0198 | OsVP1 | |
| PRO0200 | OSHI | very weak in young |
| | | plant meristem |
| PRO0208 | putative chlorophyllase | |
| PRO0210 | OsNRT1 | |
| PRO0211 | EXP3 | |
| PRO0216 | phosphate transporter OjPT1 | |
| PRO0218 | oleosin 18 kd | aleurone + embryo |
| PRO0219 | ubiquitine 2 without intron | |
| PRO0220 | RFL | |
| PRO0221 | maize UBI delta intron | not detected |
| PRO0223 | glutelin-1 | |
| PRO0224 | fragment of prolamin | |
| | RP6 promoter | |
| PRO0225 | 4xABRE | |
| PRO0226 | glutelin OSGLUA3 | |
| PRO0227 | BLZ-2_short (barley) | |
| PR00228 | BLZ-2_long (barley) | |

The inducible promoter is a promoter induced in a specific plant tissue, by a developmental stage or by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC, and MYB active in drought; the promoters INT, INPS, prxEa, Ha hsp17.7G4 and RD21 active in high salinity and osmotic stress, and the promoters hsr203J and str246C active in pathogenic stress.

According to one embodiment the promoter is a pathogen-inducible promoter. These promoters direct the expression of genes in plants following infection with a pathogen such as bacteria, fungi, viruses, nematodes and insects. Such promoters include those from pathogenesis-related proteins (PR proteins), which are induced following infection by a pathogen; e.g., PR proteins, SAR proteins, beta-1,3-glucanase, chitinase, etc. See, for example. Redolfi et al. (1983) Neth. J. Plant Pathol 89:245-254; Uknes et al. (1992) Plant Cell 4:645-656; and Van Loon (1985) Plant Mol. Virol. 4:111-116.

According to one embodiment, when more than one promoter is used in the expression vector, the promoters are identical (e.g., all identical, at least two identical).

According to one embodiment, when more than one promoter is used in the expression vector, the promoters are different (e.g., at least two are different, all are different).

According to one embodiment, the promoter in the expression vector includes, but is not limited to, CaMV 35S, 2x CaMV 35S, CaMV 19S, ubiquitin, AtU626 or TaU6.

According to a specific embodiment, the promoter in the expression vector comprises a 35S promoter.

According to a specific embodiment, the promoter in the expression vector comprises a U6 promoter.

Expression vectors may also comprise transcription and translation initiation sequences, transcription and translation terminator sequences and optionally a polyadenylation signal.

According to a specific embodiment, the expression vector comprises a termination sequence, such as but not limited to, a G7 termination sequence, an AtuNos termination sequence or a CaMV-35S terminator sequence.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et at., Theor. App. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA. (0986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

According to one embodiment, an agrobacterium-free expression method is used to introduce foreign genes into plant cells. According to one embodiment, the agrobacterium-free expression method is transient. According to a specific embodiment, a bombardment method is used to introduce foreign genes into plant cells. According to another specific embodiment, bombardment of a plant root is used to introduce foreign genes into plant cells. An exemplary bombardment method which can be used in accordance with some embodiments of the invention is discussed in the examples section which follows.

Furthermore, various cloning kits or gene synthesis can be used according to the teachings of some embodiments of the invention.

According to one embodiment the nucleic acid construct is a binary vector. Examples for binary vectors are pBIN19, pBI101, pBinAR, pGPTV, pCAMBIA, pBIB-HYG, pBecks, pGreen or pPZP (Hajukiewicz, P. et Plant Mol. Biol. 25, 989 (1994), and Hellens et al, Trends in Plant Science 5, 446 (2000)).

Examples of other vectors to be used in other methods of I)NA delivery (e.g. transfection, electroporation, bombardment, viral inoculation as discussed below) are: pGE-sgRNA (Zhang et al. Nat. Comms. 2016 7:12697), pJIT163-Ubi-Cas9 (Wang et al. Nat. Biotechnol 2004 32, 947-951), pICH47742:2x35S-5′UTR-hCas9(STOP)-NOST (Belhan et al. Plant Methods 2013 11;9(1):39), pAHC25 (Christensen, A. H. & P. H. Quail, 1996. Ubiquitin promoter-based vectors for high-level expression of selectable and/or screenable marker genes in monocotyledonous plants. Transgenic Research 5: 213-218), pHBT-sGFP(S65T)-NOS (Sheen et al. Protein phosphatase activity is required for light-inducible gene expression in maize, EMBO J. 12 (9), 3497-3505 (1993).

According to one embodiment, the method of some embodiments of the invention further comprises introducing into the plant cell donor oligonucleotides.

According to one embodiment, when the modification is an insertion, the method further comprises introducing into the plant cell donor oligonucleotides.

According to one embodiment, when the modification is a deletion, the method further comprises introducing into the plant cell donor oligonucleotides.

According to one embodiment, when the modification is a deletion and insertion (e.g. swapping), the method further comprises introducing into the plant cell donor oligonucleotides.

According to one embodiment, when the modification is a point mutation, the method further comprises introducing into the plant cell donor oligonucleotides.

As used herein, the term “donor oligonucleotides” or “donor oligos” refers to exogenous nucleotides, i.e. externally introduced into the plant cell to generate a precise change in the genome. According to one embodiment, the donor oligonucleotides are synthetic.

According to one embodiment, the donor oligos are RNA oligos.

According to one embodiment, the donor oligos are DNA oligos.

According to one embodiment, the donor oligos are synthetic oligos.

According to one embodiment, the donor oligonucleotides comprise single-stranded donor oligonucleotides (ssODN).

According to one embodiment, the donor oligonucleotides comprise double-stranded donor oligonucleotides (dsODN).

According to one embodiment, the donor oligonucleotides comprise double-stranded DNA (dsDNA).

According to one embodiment, the donor oligonucleotides comprise double-stranded DNA-RNA duplex (DNA-RNA duplex).

According to one embodiment, the donor oligonucleotides comprise double-stranded DNA-RNA hybrid.

According to one embodiment, the donor oligonucleotides comprise single-stranded DNA-RNA hybrid.

According to one embodiment, the donor oligonucleotides comprise single-stranded DNA (ssDNA).

According to one embodiment, the donor oligonucleotides comprise double-stranded RNA (dsRNA).

According to one embodiment, the donor oligonucleotides comprise single-stranded RNA (ssRNA).

According to one embodiment, the donor oligonucleotides comprise the DNA or RNA sequence for swapping (as discussed above).

According to one embodiment, the donor oligonucleotides are provided in a non-expressed vector format or oligo.

According to one embodiment, the donor oligonucleotides comprise a DNA donor plasmid (e.g. circular or linearized plasmid).

According to one embodiment, the donor oligonucleotides comprise about 50-5000, about 100-5000, about 250-5000, about 500-5000, about 750-5000, about 1000-5000, about 1500-5000, about 2000-5000, about 2500-5000, about 3000-5000, about 4000-5000, about 50-4000, about 100-4000, about 250-4000, about 500-4000, about 750-4000, about 1000-4000, about 1500-4000, about 2000-4000, about 2500-4000, about 3000-4000, about 50-3000, about 100-3000, about 250-3000, about 500-3000, about 750-3000, about 1000-3000, about 1500-3000, about 2000-3000, about 50-2000, about 100-2000, about 250-2000, about 500-2000, about 750-2000, about 1000-2000, about 1500-2000, about 50-1000, about 100-1000, about 250-1000, about 500-1000, about 750-1000, about 50-750, about 150-750, about 250-750, about 500-750, about 50-500, about 150-500, about 200-500, about 250-500, about 350-500, about 50-250, about 150-250, or about 200-250 nucleotides.

According to a specific embodiment, the donor oligonucleotides comprising the ssODN (e.g. ssDNA or ssRNA) comprise about 200-500 nucleotides.

According to a specific embodiment, the donor oligonucleotides comprising the dsODN (e.g. dsDNA or dsRNA) comprise about 250-5000 nucleotides.

According to one embodiment, for gene swapping of an endogenous RNA silencing molecule (e.g. miRNA) with a RNA silencing sequence of choice (e,g. siRNA), the expression vector, ssODN (e.g. ssDNAA or ssRNA) or dsODN (e.g. dsDNA or dsRNA) does not have to be expressed in a plant cell and could serve as a non-expressing template. According to a specific embodiment, in such a case only the DNA editing agent (e.g. Cas9/sgRNA modules) need to be expressed if provided in a DNA form.

According to some embodiments, for gene editing of an endogenous RNA molecule (e.g., RNA silencing molecule) without the use of a nuclease, the DNA editing agent (e.g., sgRNA) may be introduced into the eukaryotic cell with orour without (e.g. oligonucleotide donor DNA or RNA, as discussed herein).

According to one embodiment, introducing into the plant cell donor oligonucleotides is effected using any of the methods described above (e.g. using the expression vectors or RNP transfection).

According to one embodiment, the sgRNA and the DNA donor oligonucleotides are co-introduced into the plant cell (e.g. via bombardment). It will be appreciated that any additional factors (e.g. nuclease) may be co-introduced therewith.

According to one embodiment, the sgRNA is introduced into the plant cell prior to the DNA donor oligonucleotides (e.g. within a few minutes or a few hours). It will be appreciated that any additional factors (e.g. nuclease) may be introduced prior to, concomitantly with, or following the sgRNA or the DNA donor oligonucleotides.

According to one embodiment, the sgRNA is introduced into the plant cell subsequent to the DNA donor oligonucleotides within a few minutes or a few hours). It will be appreciated that any additional factors (e.g. nuclease) may be introduced prior to, concomitantly with, or following the sgRNA or the DNA donor oligonucleotides.

According to one embodiment, there is provided a composition comprising at least one sgRNA and DNA donor oligonucleotides for genome editing.

According to one embodiment, there is provided a composition comprising at least one sgRNA, a nuclease (e.g. endonuclease) and. DNA donor oligonucleotides for genome editing.

There are various methods of direct DNA transfer into plant cells and the skilled artisan will know which to select. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or gold or tungsten particles, and the microprojectiles are physically accelerated into protoplasts, cells or plant tissues.

Thus, the delivery of nucleic acids may be introduced into a plant cell in embodiments of the invention by any method known to those of skill in the art, including, for example and without limitation: by transformation of protoplasts (See, e.g., U.S. Pat. No. 5,508,184); by desiccation/inhibition-mediated DNA uptake (See, e.g., Potrykus et al. (1985) Mol. Gen. Genet, 199:183-8); by electroporation (See, e.g., U.S. Pat. No. 5,384,253); by agitation with silicon carbide fibers (See, e.g., U.S. Pat. Nos. 5,302,523 and 5,464,765); by Agrobacterium-mediated transformation (See, e.g., U.S. Pat. Nos. 5,563,055, 5,591,616, 5,693,512, 5,824,877, 5,981,840, and 6,384,301); by acceleration of DNA-coated particles (See, e.g., U.S. Pat. Nos. 5,015,580, 5,550,318, 5,538,880, 6,160,208, 6,399,861, and 6,403,865) and by Nanoparticles, nanocarriers and cell penetrating peptides (WO201126644A2, WO2009046384A1; WO2008148223A1) in the methods to deliver DNA, RNA, Peptides and/or proteins or combinations of nucleic acids and peptides into plant cells.

Other methods of transfection include the use of transfection reagents (e.g. Lipofectin, ThermoFisher), dendrimers (Kukowska-Latallo, J. F. et al., 1996, Proc. Natl. Acad. Sci. USA93, 4897-902), cell penetrating peptides (Mäe et al., 2005, Internalisation of cell-penetrating peptides into tobacco protoplasts, Biochimica et Biophysica Acta 1669(2):101-7) or polyamines (Zhang and Vinogradov, 2010, Short biodegradable polyamines for gene delivery and transfection of brain capillary endothelial cells, J Control Release, 143(3):359-366).

According to a specific embodiment, for introducing DNA into plant cells (e.g. protoplasts) the method comprises polyethylene glycol (PEG)-mediated DNA uptake. For further details see Karesch et al. (1991) Plant Cell Rep. 9:575-578; Mathur et al. (1995) Plant Cell Rep. 14:221-226; Negrutiu et al. (1987) Plant Cell Mol. Biol. 8:363-373. Plant cells (e.g. protoplasts) are then cultured under conditions that allowed them to grow cell walls, start dividing to form a callus, develop shoots and roots, and regenerate whole plants.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the genetically identical transformed plants.

Micropropagation is a process of growing new generation plants from a single piece of tissue that has been excised from a selected parent plant or cultivar. This process permits the mass reproduction of plants having the desired trait. The new generated plants are genetically identical to, and have all of the characteristics of, the original plant. Micropropagation (or cloning) allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV, TRV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (B V); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is a RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleicacid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that the sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Regardless of the transformation/infection method employed, the present teachings further select transformed cells comprising a genome editing event.

According to a specific embodiment, selection is carried out such that only cells comprising a successful accurate modification (e.g. swapping, insertion, deletion, point mutation) in the specific locus are selected. Accordingly, cells comprising any event that includes a modification (e.g. an insertion, deletion, point mutation) in an unintended locus are not selected.

According to one embodiment, selection of modified cells can be performed at the phenotypic level, by detection of a molecular event, by detection of a fluorescent reporter, or by growth in the presence of selection (e.g., antibiotic).

According to one embodiment, selection of modified cells is performed by analyzing the biogenesis and occurrence of the newly generated dsRNA molecule.

According to one embodiment, selection of modified cells is performed by analyzing the biogenesis and occurrence of secondary small RNAs (generated by further processing of the dsRNA).

According to one embodiment, selection of modified cells is performed by analyzing the biogenesis and occurrence of the newly edited RNA molecule (e.g. the presence of new miRNA version, the presence of novel edited siRNAs, piRNAs, tasiRNAs etc).

According to one embodiment, selection of modified cells is performed by analyzing the biogenesis and occurrence of the newly edited plant RNA transcripts (i.e. of the modified plant gene).

According to one embodiment, selection of modified cells is performed by analyzing the silencing activity and/or specificity of the modified RNA molecule (e.g. RNA silencing molecule) or of the modified plant RNA towards a plant RNA or pest RNA, respectively, or the silencing activity and/or specificity of the dsRNA molecule or secondary small RNAs processed therefrom towards a pest RNA, by validating at least one phenotype in the plant (e.g. plant leaf coloring, e.g. partial or complete loss of chlorophyll in leaves and other organs (bleaching), presence/absence of necrotic patterns, flower coloring, fruit traits (such as shelf life, firmness and flavor), growth rate, plant size (e.g. dwarfism), crop yield, biotic stress resistance) or in the pest (e.g. nematode mortality, beetle's egg laying rate, or other resistant phenotypes associated with any of bacteria, viruses, fungi, parasites, insects, weeds, and cultivated or native plants).

According to one embodiment, the silencing specificity of the RNA molecule, the plant RNA, the dsRNA, or the secondary small RNAs processed therefrom, is determined genotypically, e.g. by expression of a gene or lack of expression.

According to one embodiment, the silencing specificity of the RNA molecule, the plant RNA, the dsRNA or secondary small RNAs processed therefrom, is determined phenotypically.

According to one embodiment, a phenotype of the plant is determined prior to a genotype.

According to one embodiment, a genotype of the plant is determined prior to a phenotype.

According to one embodiment, selection of modified cells is performed by analyzing the silencing activity and/or specificity of the RNA molecule (e.g. RNA silencing molecule), the plant RNA, the dsRNA or the secondary small RNAs processed therefrom, towards a plant RNA or pest RNA by measuring a RNA level of the plant RNA or pest RNA. This can be performed using any method known in the art, e.g. by Northern blotting, Nuclease Protection Assays, In Situ hybridization, or quantitative RT-PCR.

According to one embodiment, selection of modified cells is performed by analyzing plant cells or clones comprising the DNA editing event also referred to herein as “mutation” or “edit”, dependent on the type of editing sought e.g., insertion, deletion, insertion-deletion andel), inversion, substitution and combinations thereof.

Methods for detecting sequence alteration are well known in the art and include, but not limited to, DNA and RNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Heteroduplex and Sanger sequencing, or PCR followed by restriction digest to detect appearance or disappearance of unique restriction site/s.

Another method of validating the presence of a DNA editing event e.g., Indels comprises a mismatch cleavage assay that makes use of a structure selective enzyme (e.g. endonuclease) that recognizes and cleaves mismatched DNA.

According to one embodiment, selection of transformed cells is effected by flow cytometry (FACS) selecting transformed cells exhibiting fluorescence emitted by the fluorescent reporter. Following FACS sorting, positively selected pools of transformed plant cells, displaying the fluorescent marker are collected and an aliquot can be used for testing the DNA editing event as discussed above.

In cases where antibiotic selection marker was used, following transformation plant cell clones are cultivated in the presence of selection (e.g., antibiotic) until they develop into colonies i.e., clones and micro-calli. A portion of the cells of the calli are then analyzed (validated) for the DNA editing event, as discussed above.

Thus, according to one embodiment of the invention, the method further comprises validating in the transformed cells complementarity of the RNA molecule (e.g. RNA silencing molecule), the plant RNA, the dsRNA or the secondary small RNAs processed therefrom, towards the plant RNA or pest RNA.

As mentioned above, following modification, the RNA molecule (e.g. RNA silencing molecule) the plant RNA, the dsRNA (e.g. sense or anti-sense strand thereof) or secondary small RNAs processed therefrom, comprises at least about 30%, 33%, 40%, 50%, 60%, 70%, 80%, 85%, 90%, 91%, 92%, 93%, 94%, 95%, 96%, 97%, 98%, 99% or even 100% complementarity towards the target sequence of the plant RNA or pest RNA.

The specific binding of designed RNA molecule with a target plant RNA or pest RNA can be determined by any method known in the art, such as by computational algorithms (e.g. BLAST) and verified by methods including e,g. Northern blot, In Situ hybridization, QuantiGene Plex Assay etc.

It will be appreciated that positive clones can be homozygous or heterozygous for the DNA editing event. In case of a heterozygous cell, the cell (e.g., when diploid) may comprise a copy of a modified gene and a copy of a non-modified gene. The skilled artisan will select the clone for further culturing/regeneration according to the intended use.

According to one embodiment, when a transient method is desired, clones exhibiting the presence of a DNA editing event as desired are further analyzed and selected for the absence of the DNA editing agent, namely, loss of DNA sequences encoding for the DNA editing agent. This can be done, for example, by analyzing the loss of expression of the DNA editing agent (e.g., at the mRNA, protein) e.g., by fluorescent detection of CiFP or q-PCR, HPLC.

According to one embodiment, when a transient method is desired, the cells may be analyzed for the absence of the nucleic acid construct as described herein or portions thereof e.g., nucleic acid sequence encoding the DNA editing agent. This can be affirmed by fluorescent microscopy, q-PCR, FACS, and or any other method such as Southern blot, PCR, sequencing, HPLC).

According to one embodiment, the plant is crossed in order to obtain a plant devoid of the DNA editing agent (e.g. of the endonuclease), as discussed below.

Positive clones may be stored (e.g., cryopreserved).

Alternatively, plant cells (e.g., protoplasts) may be regenerated into whole plants first by growing into a group of plant cells that develops into a callus and then by regeneration of shoots (callogenesis) from the callus using plant tissue culture methods. Growth of protoplasts into callus and regeneration of shoots requires the proper balance of plant growth regulators in the tissue culture medium that must be customized for each species of plant.

Protoplasts may also be used for plant breeding, using a technique called protoplast fusion. Protoplasts from different species are induced to fuse by using an electric field or a solution of polyethylene glycol. This technique may be used to generate somatic hybrids in tissue culture.

Methods of protoplast regeneration are well known in the art. Several factors affect the isolation, culture, and regeneration of protoplasts, namely the genotype, the donor tissue and its pre-treatment, the enzyme treatment for protoplast isolation, the method of protoplast culture, the culture, the culture medium, and the physical environment. For a thorough review see Maheshwari et al. 1986 Differentiation of Protoplasts and of Transformed Plant Cells: 3-36. Springer-Verlag, Berlin.

The regenerated plants can be subjected to further breeding and selection as the skilled artisan sees fit.

Thus, embodiments of the invention further relate to plants, plant cells and processed product of plants comprising the dsRNA molecule capable of silencing a pest gene according to the present teachings.

According to one aspect of the invention, there is provided a method of generating a pest tolerant or resistant plant, the method comprising producing a long dsRNA molecule capable of silencing a pest gene in a plant cell according to the method of some embodiments of the invention.

According to one aspect of the invention, there is provided a method of producing a pest tolerant or resistant plant, the method comprising:

(a) breeding the plant some embodiments of the invention; and

(b) selecting for progeny plants that express the long dsRNA molecule capable of suppressing the pest gene, and which do not comprise the DNA editing agent,

thereby producing the pest tolerant or resistant plant.

According to one aspect of the invention, there is provided a method producing a plant or plant cell of some embodiments of the invention comprising growing the plant or plant cell under conditions which allow propagation.

According to one embodiment, breeding comprises crossing or selfing.

The term “crossing” as used herein refers to the fertilization of female plants (or gametes) by male plants (or gametes). The term “gamete” refers to the haploid reproductive cell (egg or sperm) produced in plants by mitosis from a gametophyte and involved in sexual reproduction, during which two gametes of opposite sex fuse to form a diploid zygote. The term generally includes reference to a pollen (including the sperm cell) and an ovule (including the ovum). “crossing” therefore generally refers to the fertilization of ovules of one individual with pollen from another individual, whereas “selfing” refers to the fertilization of ovules of an individual with pollen from the same individual. Crossing is widely used in plant breeding and results in a mix of genomic information between the two plants crossed one chromosome from the mother and one chromosome from the father. This will result in a new combination of genetically inherited traits.

As mentioned above, the plant may be crossed in order to obtain a plant devoid of undesired factors e.g. DNA editing agent (e.g. endonuclease).

According to some embodiments of the invention, the plant is non-transgenic.

According to some embodiments of the invention, the plant is a transgenic plant.

According to one embodiment, the plant is non-genetically modified (non-GMO).

According to one embodiment, the plant is genetically modified (GMO).

According to one aspect of the invention, there is provided a cell of the plant of some embodiments of the invention.

According to one aspect of the invention, there is provided a seed of the plant of some embodiments of the invention.

According to one embodiment, the plants generated by the present method are more resistant or tolerant to pests by at least about 10%, 20%, 30%, 40%, 50%, 60%, 70%, 80%, 90%, 95% or 100% as compared to plants not generated by the present methods (i.e. as compared to wild type plants).

Any method known in the art for assessing tolerance or resistance to pests of a plant may be used in accordance with the present invention. Exemplary methods include, but are not limited to, reducing MYB46 expression in Arabidopsis which results in enhanced resistance to Botrytis cinereal as described in Ramirez V1, Garcia-Andrade J, Vera P., Plant Signal Behay. 2011 Jun.;6(6):911-3, Epub 2011 Jun. 1; or downregulation of HCT in alfalfa promotes activation of defense response in the plant as described in Gallego-Giraldo L. et al. New Phytologist (2011) 190: 627-639 doi: 10.1111/j.1469-8137.2010.03621.x), both incorporated herein by reference.

According to further embodiments, there is provided a method of producing a long dsRNA molecule in a plant cell, wherein the long dsRNA is capable of silencing a target gene of interest, the method comprising: (a) selecting a first nucleic acid sequence of a plant gene exhibiting a predetermined sequence homology to a nucleic acid sequence of the target gene of interest; and (b) modifying a second plant endogenous nucleic acid sequence encoding an RNA molecule so as to impart silencing specificity towards the first plant gene, such that small RNA molecules capable of recruiting RNA-dependent RNA Polymerase (RdRp) processed from the RNA molecule form base complementation with a transcript of the first plant gene to produce the long dsRNA molecule capable of silencing the target gene of interest.

According to some embodiments, the first nucleic acid sequence does not encode for a silencing RNA prior to use of the above method. According to some embodiments, the long dsRNA is not naturally produced from the first nucleic acid sequence prior to use of the above method. Without wishing to be bound by theory or mechanism, while the first nucleic acid sequence in the above method does not necessarily produce long dsRNA naturally (or any silencing RNA), modification of the second plant endogenous nucleic acid sequence results in an RNA molecule (e.g. a miRNA) which acts as an amplifier and engages RdRp to generate long dsRNA from an RNA transcript of the first nucleic acid sequence. Thus, in effect, the above method is able, according to some embodiments, to generate a long dsRNA from a gene which previously did not produce one.

According to some embodiments, the target gene of interest is an endogenous gene of the plant cell. According to other embodiments, the target gene of interest is an exogenous gene to the plant cell (e.g. a gene of a pest, e.g. invertebrate pest).

According to some embodiments, the RNA molecule encoded by the second plant endogenous nucleic acid sequence is a miRNA.

According to some embodiments, the predetermined sequence homology to a nucleic acid sequence of the target gene of interest comprises homology of at least two stretches of at least 28 nt each, each having at least 90% homology to the sequence of the target gene of interest.

According to some embodiments, modifying a nucleic acid sequence comprises using a DNA editing agent, such as, but not limited to, a CRISPR-endonuclease (e.g. Cas9). According to some embodiments, the DNA editing agent comprises a CRIPSR-endonuclease and a guide RNA directed at cutting a nucleic acid sequence of interest (e.g. the sequence of the second plant endogenous nucleic acid). According to some embodiments, modifying a nucleic acid sequence of interest comprises using a DNA editing agent (possibly with a guide RNA directed at cutting the nucleic acid of interest) and further introducing into the plant cell an additional nucleic acid sequence which is similar to the nucleic acid sequence to be modified but includes the desired nucleotide changes. Without wishing to be bound by theory or mechanism, the DNA editing agent cuts the nucleic acid sequence of interest and part of the additional nucleic acid sequence (which includes the desired nucleotide changes) is introduced into the nucleic acid sequence of interest via. Homology Dependent Recombination (HDR).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence that corresponds to a nucleic acid sequence, or the RNA sequence of an RNA molecule nucleic acid sequence. Similarly, though some sequences are expressed in a RNA sequence format (e.g, reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological, microscopy and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected. Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

General Materials and Experimental Procedures

Computational Pipeline to Generate GEiGS Templates

The computational GEiGS pipeline applies biological metadata and enables an automatic generation of GEiGS DNA templates that are used to minimally edit non-coding RNA genes (e.g. miRNA genes), leading to a new gain of function. i.e. redirection of their silencing capacity to target sequence of interest.

As illustrated in FIG. 6, the pipeline starts with filling and submitting input: a) target sequence to be silenced by GEiGS; b) the host organism to be gene edited and to express the GEiGS; c) one can choose whether the GEiGS would be expressed ubiquitously or not. If specific GEiGS expression is required, one can choose from a few options (expression specific to a certain tissue, developmental stage, stress, heat/cold shock etc).

When all the required input is submitted, the computational process begins with searching among miRNA datasets (e.g. small RNA sequencing, microarray etc.) and filtering only relevant miRNAs that match the input criteria. Next, the selected mature miRNA sequences are aligned against the target sequence and miRNA with the highest complementary levels are filtered. These naturally target-complementary mature miRNA sequences are then modified to perfectly match the target's sequence. Then, the modified mature miRNA sequences are run through an algorithm that predicts siRNA potency and the top 20 with the highest silencing score are filtered. These final modified miRNA genes are then used to generate 200-500 nt ssDNA or 250-5000 nt dsDNA sequences as follows:

200-500 nt ssDNA oligos and 250-5000 nt dsDNA fragments are designed based on the genomic DNA sequence that flanks the modified miRNA. The pre-miRNA sequence is located in the center of the oligo. The modified miRNA's guide strand (silencing) sequence is 100% complementary to the target. However, the sequence of the modified passenger miRNA strand is further modified to preserve the original (unmodified) miRNA. structure, keeping the same base pairing profile.

Next, differential sgRNAs are designed to specifically target the original unmodified miRNA gene, and not the modified swapping version. Finally, comparative restriction enzyme site analysis is performed between the modified and the original miRNA gene and differential restriction sites are summarized.

Therefore, the pipeline output includes:

a) 200-500 nt ssDNA oligo or 250-5000 nt dsDNA fragment sequence with minimally modified miRNA

b) 2-3 differential sgRNAs that target specifically the original miRNA gene and not the modified

c) List of differential restriction enzyme sites among the modified and original miRNA gene.

Design of dsRNA by GEiGS

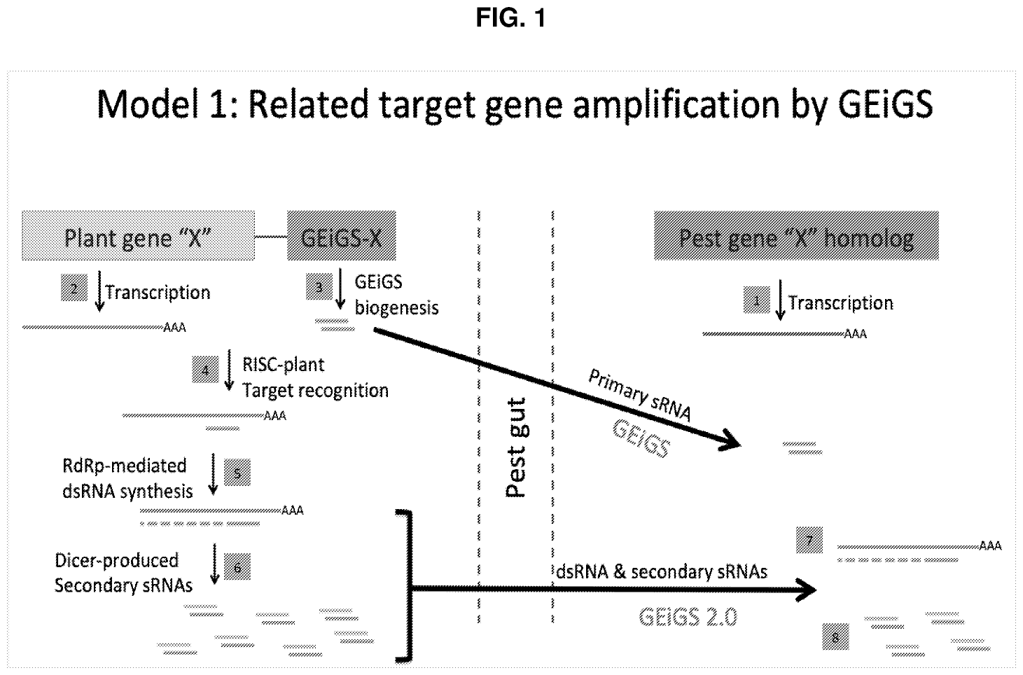

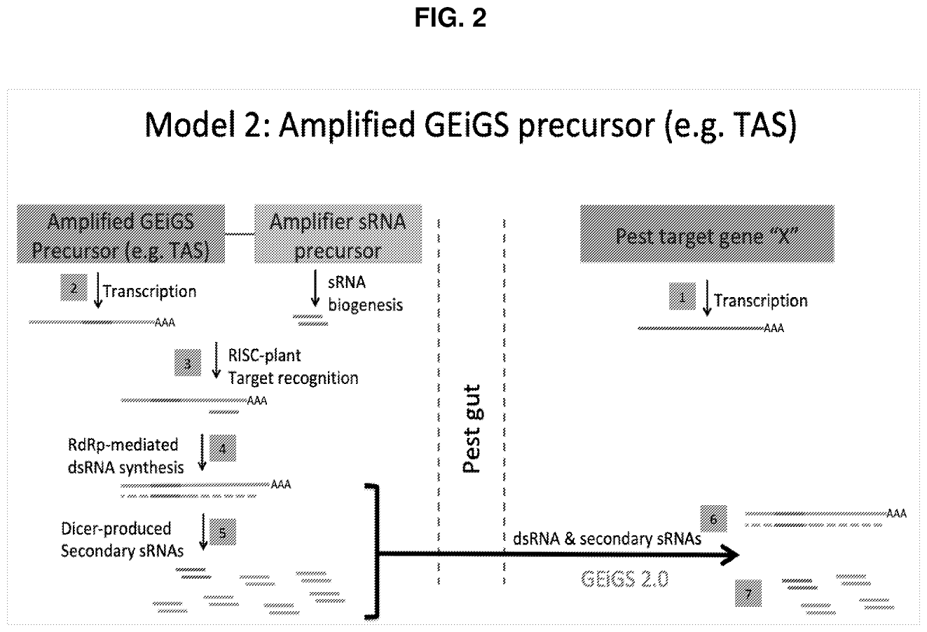

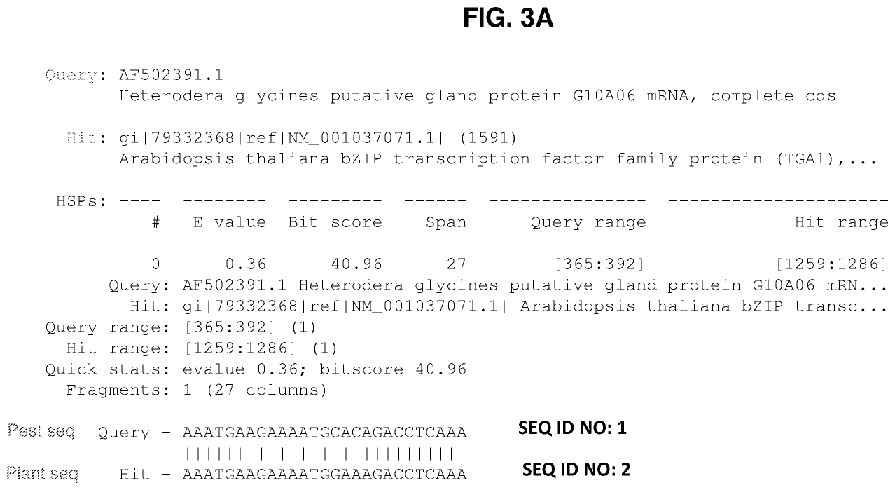

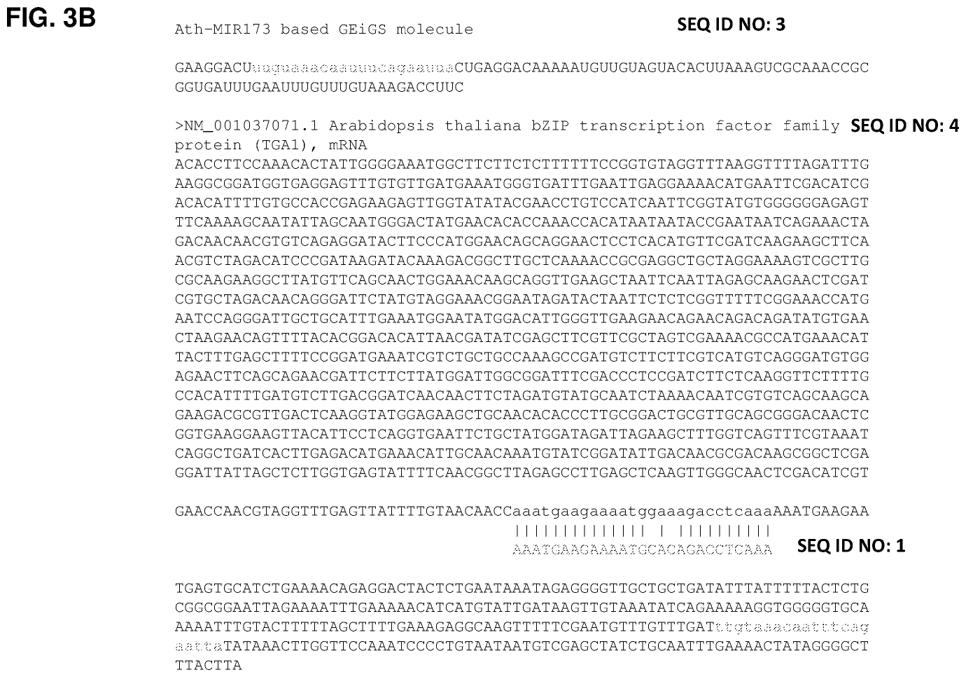

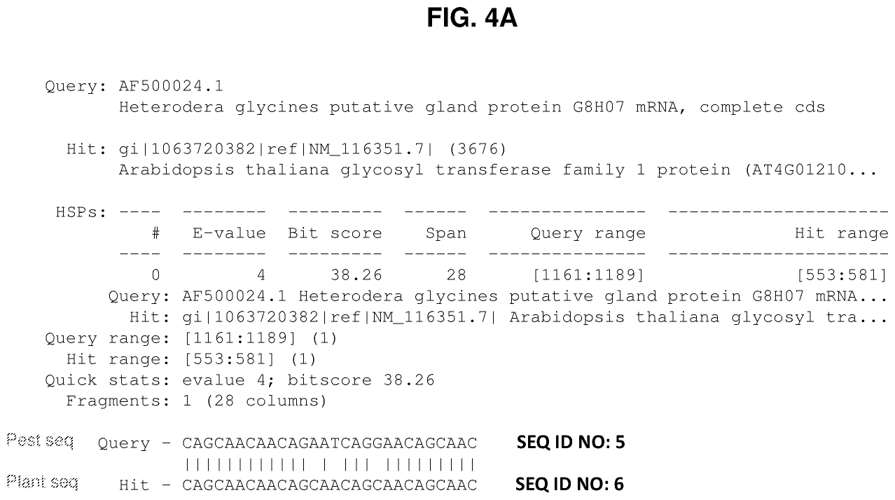

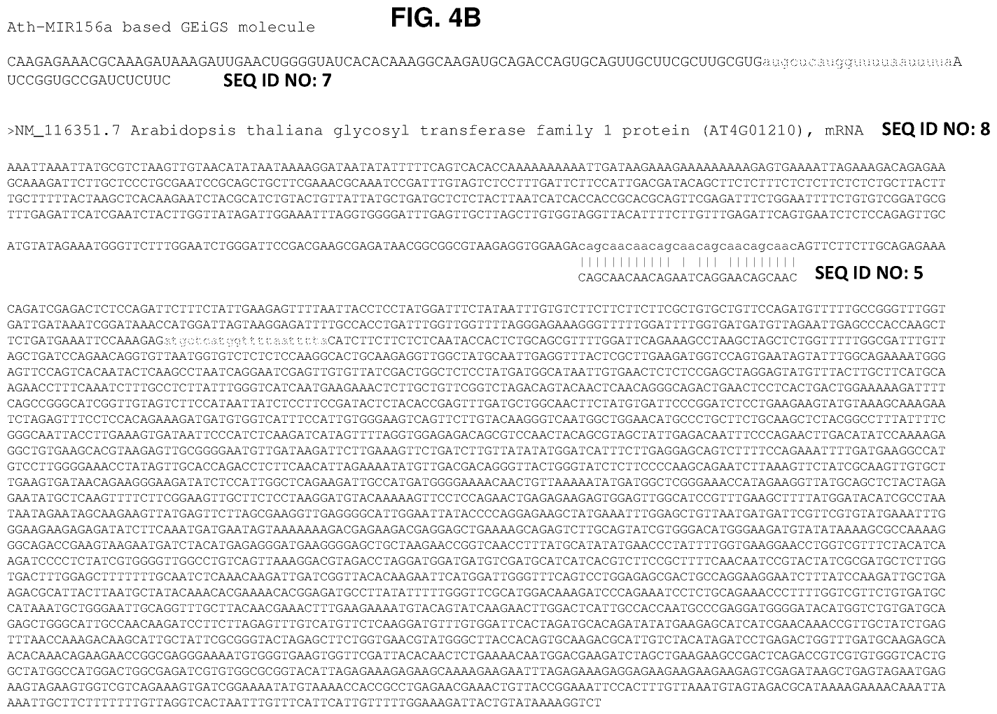

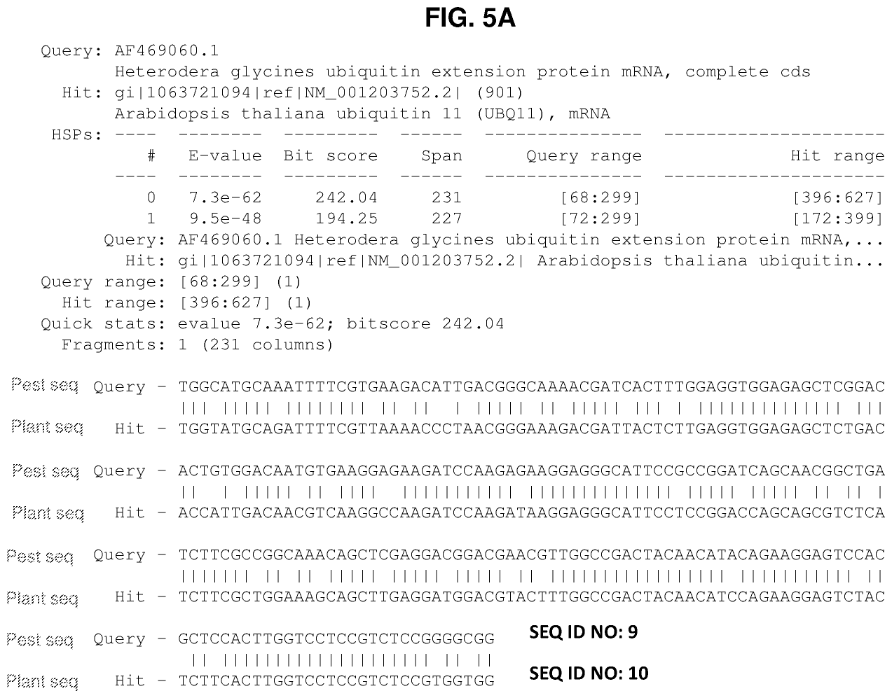

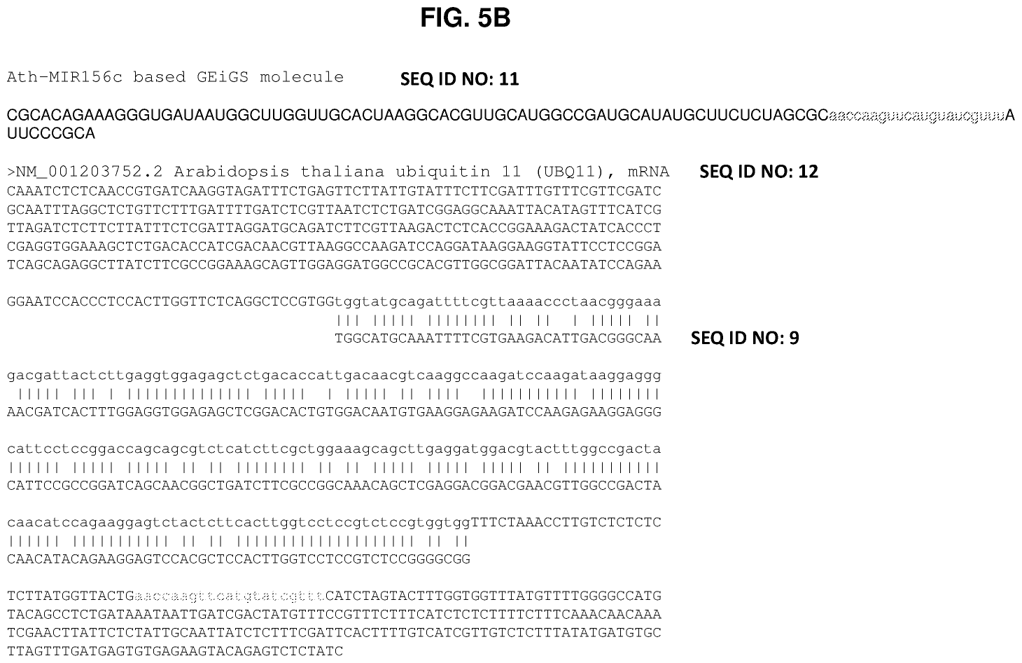

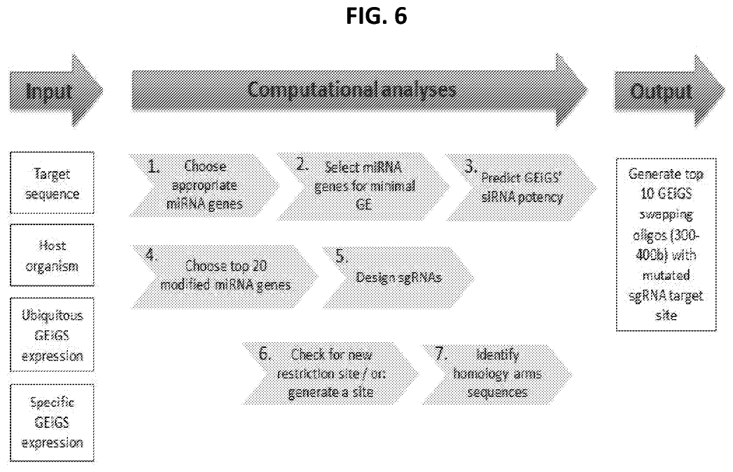

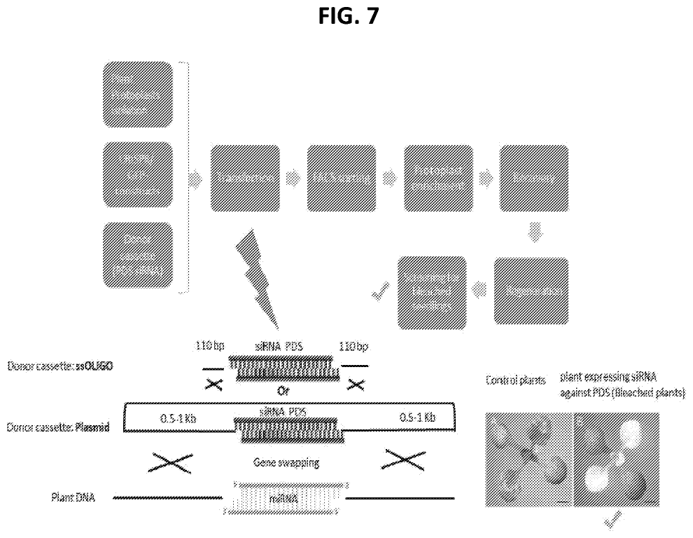

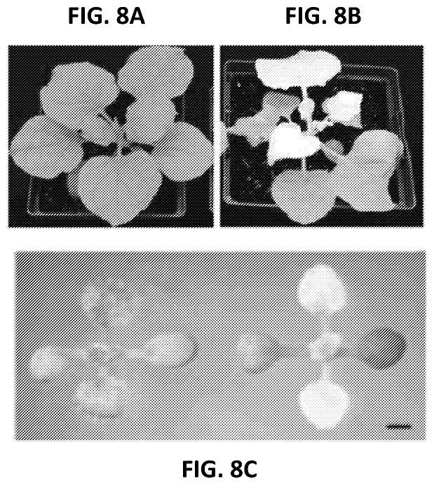

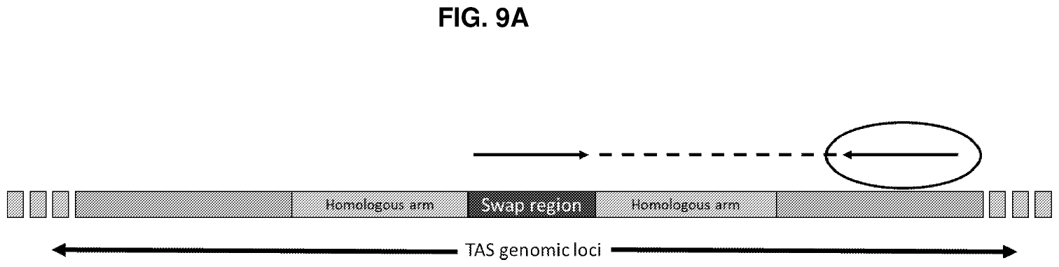

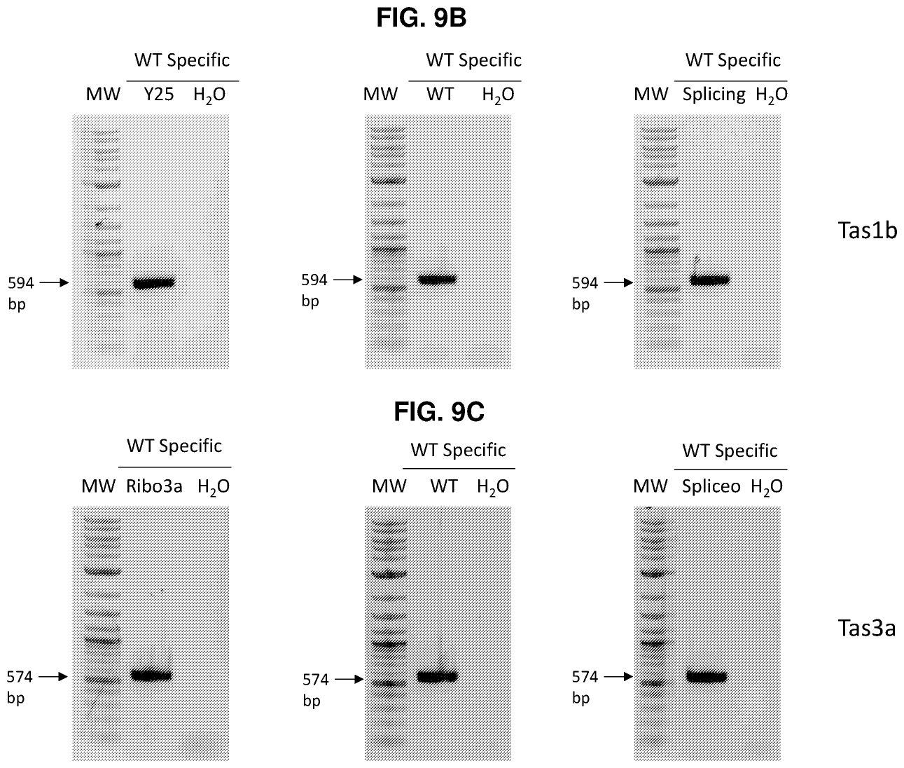

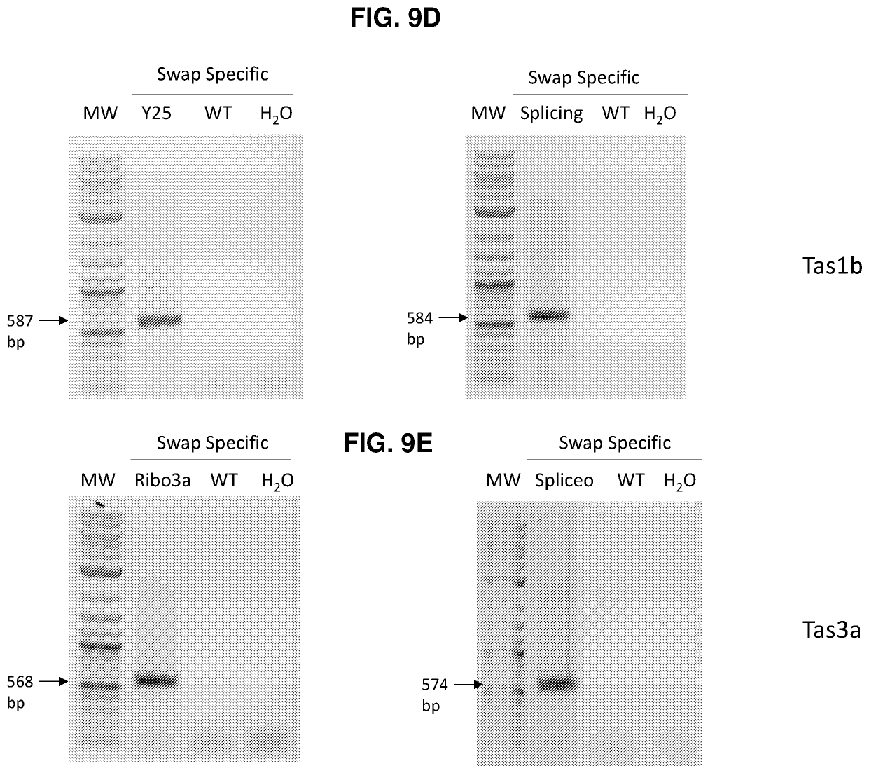

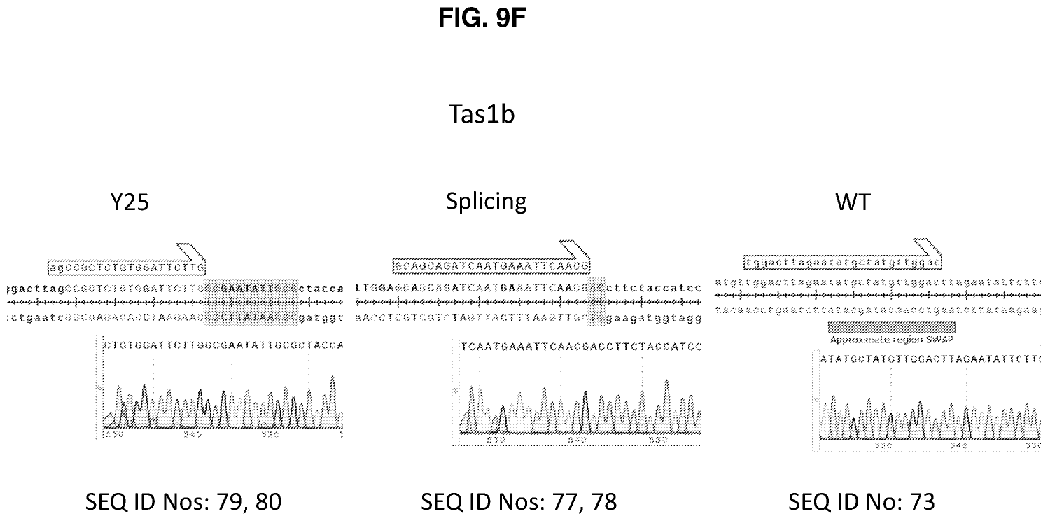

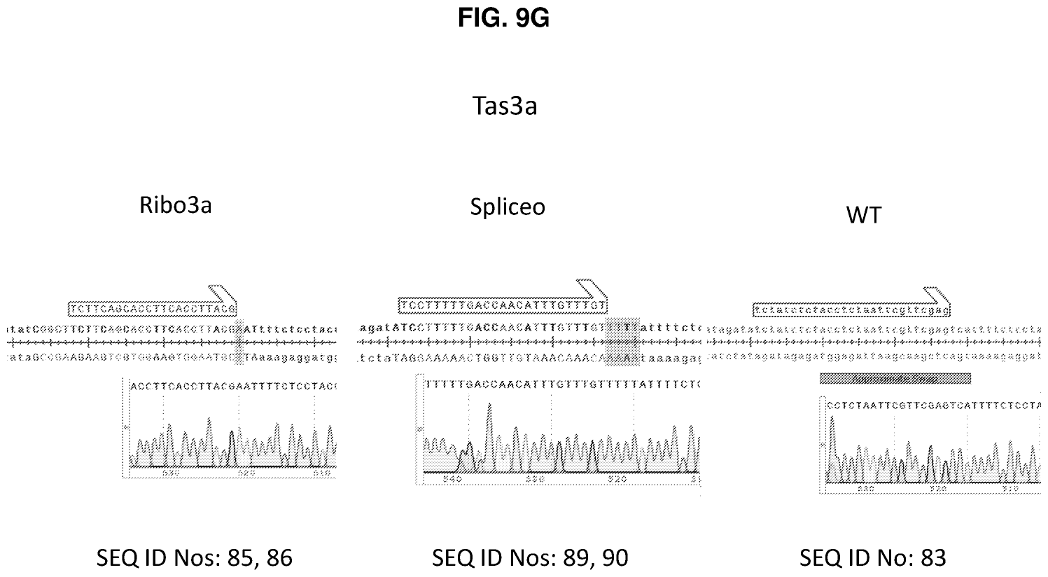

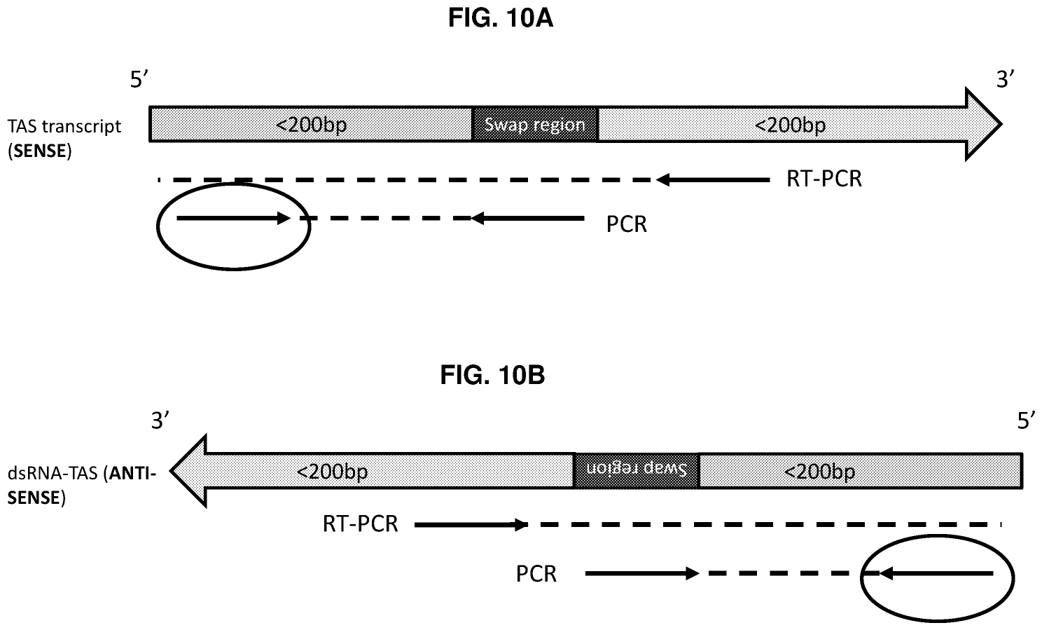

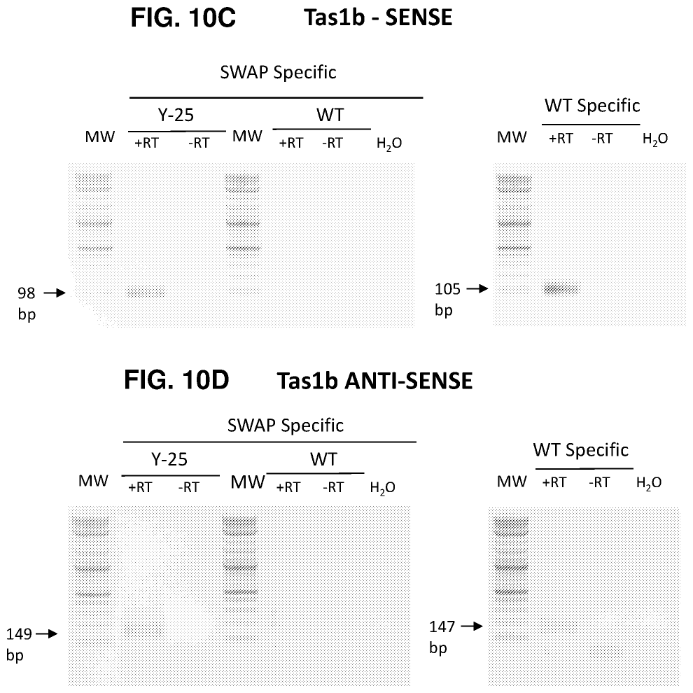

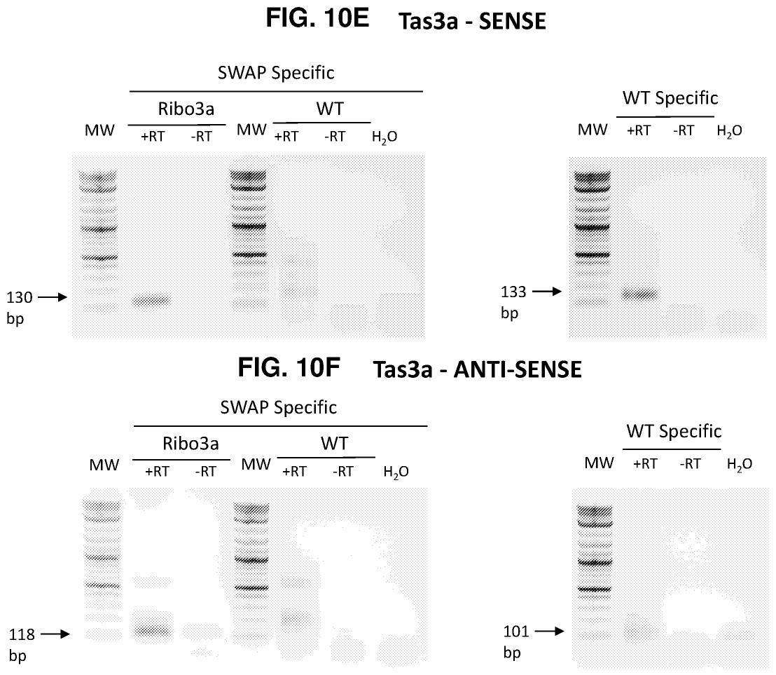

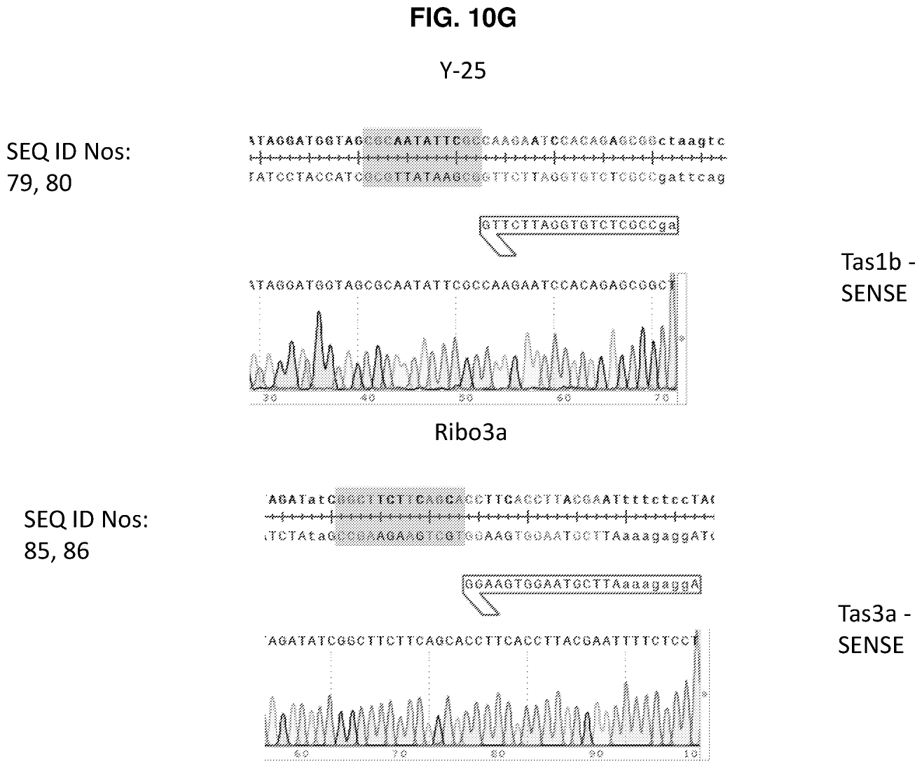

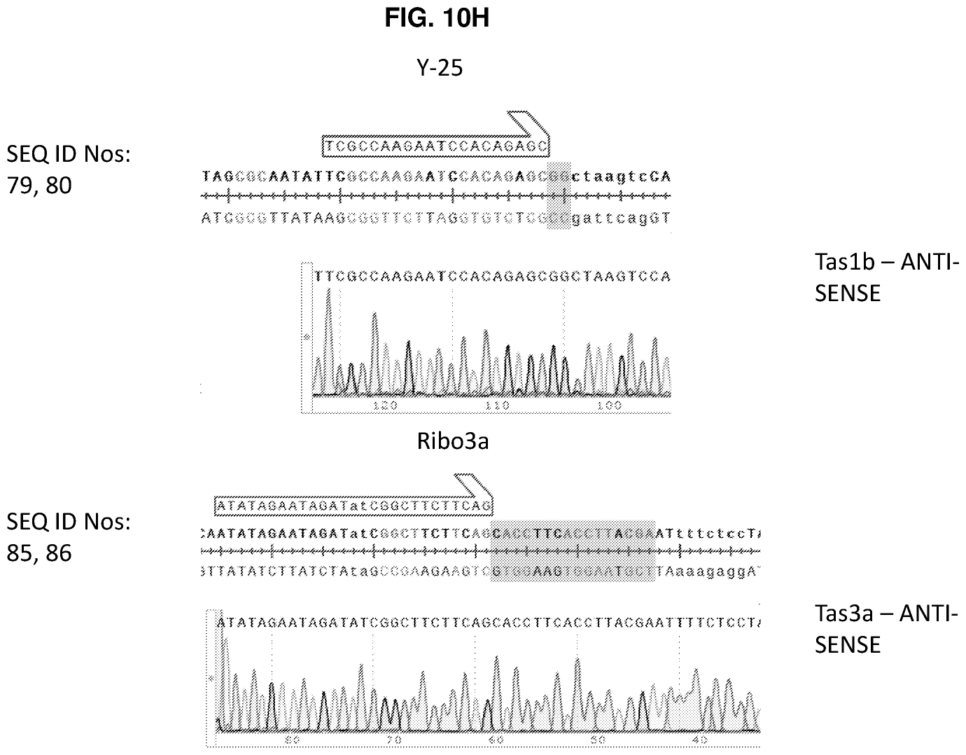

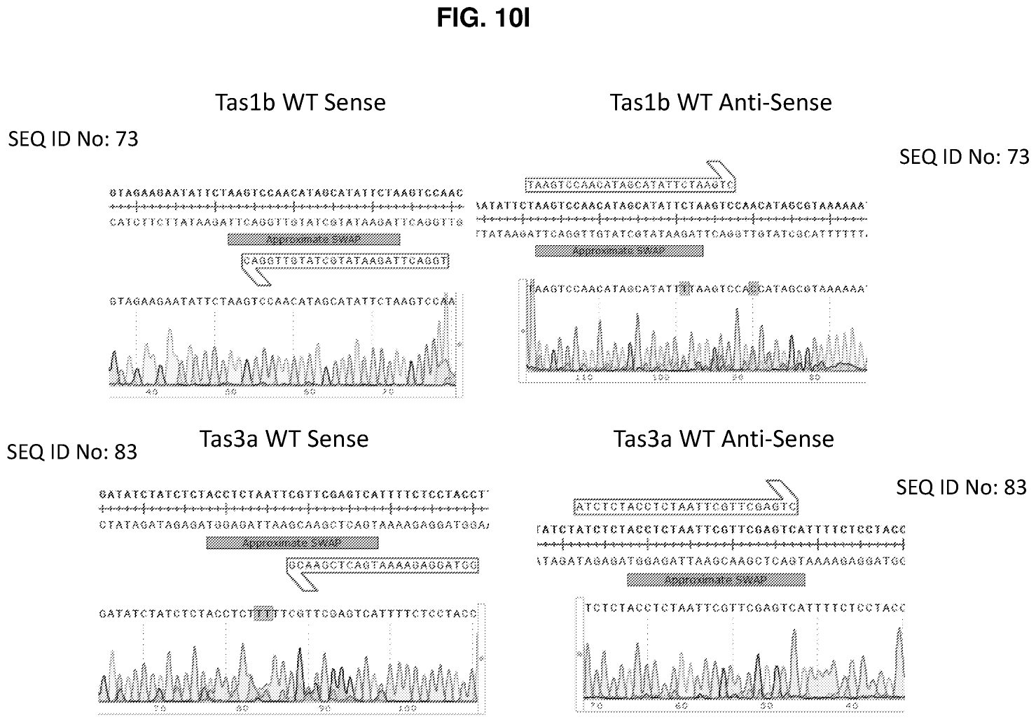

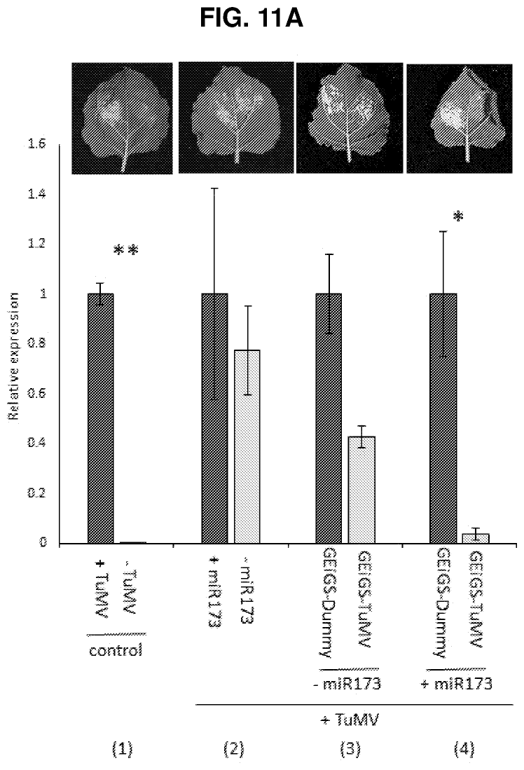

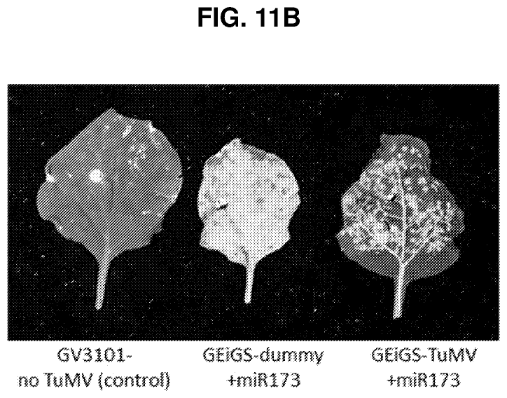

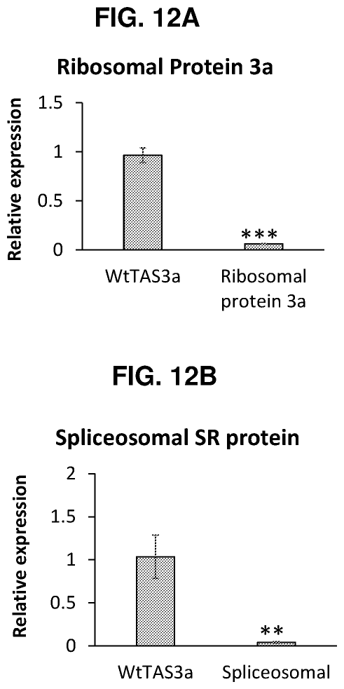

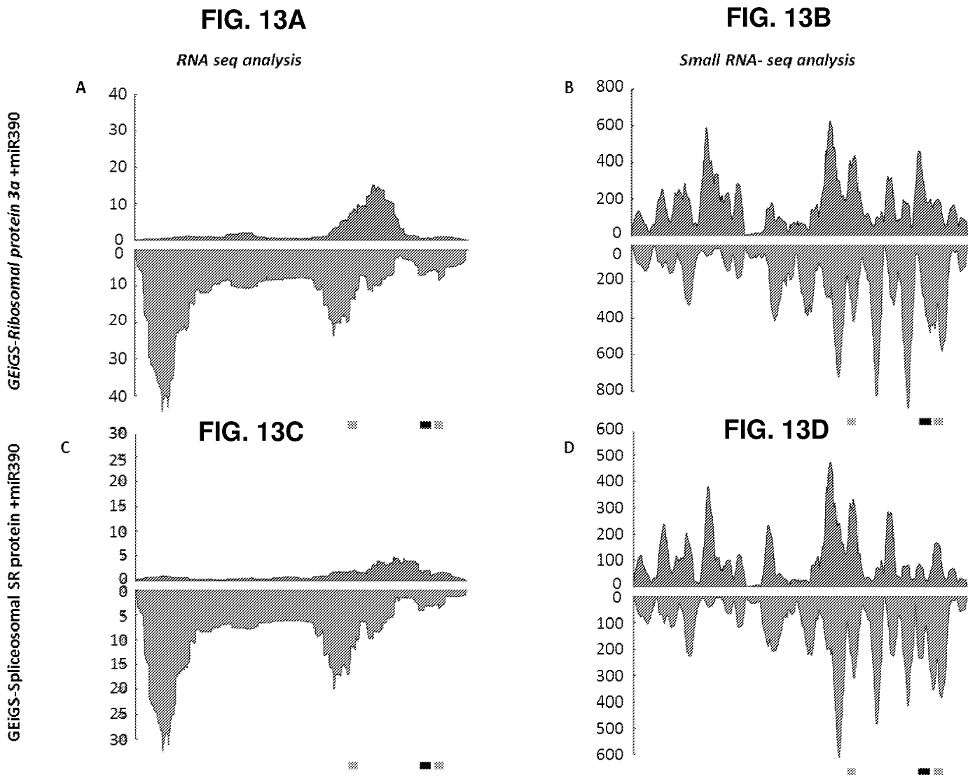

Model 1 (the numbers correspond to the numbers in FIG. 1):

1. The pest gene “X” is the target gene (when silenced, the pest is controlled)

2. A host-related gene-X is identified by homology search to pest gene “X” (plant gene “X”). According to some embodiments, the plant gene X is identified according to model 1, if it comprises at least two stretches of at least 28 nt, each having at least 90% homology to the sequence of pest gene X.

3. GEiGS is performed within plant cells in order to redirect the silencing specificity of a small RNA molecule (e.g. 22 nt miRNAs) towards host-related gene-X, thereby the small RNA molecule acts as an amplifier of RdRp-mediated transcription fur the transcript of plant gene “X”.

4. The amplifier small RNA, whose silencing specificity has been redirected using GEiGS (also referred to herein as “small GEiGS RNA”) forms a RISC complex that is associated with RdRp (the amplifying enzyme)

5. The RdRp synthesizes a complementary antisense RNA strand to the transcript of plant gene “X”, forming a long dsRNA.

6. The long dsRNA is then at least partially processed into secondary sRNAs by dicer(s) or other nucleases within the plant cells. Out of these secondary sRNAs, the silencing specificity of some of the secondary sRNA is towards pest gene X.

7. The dsRNA is also at least partly taken up by pests, possibly being processed in the pest to sRNAs, as described above.

8. Possibly, secondary sRNAs from the plant cells are also taken up by pests and also silence the target gene “X”, e.g. in addition to the generated long dsRNA.

Model 2 (the numbers correspond to the numbers in FIG. 2):

1. The pest gene “X” is the target gene (when silenced, the pest is controlled)

2. GEiGS is performed in plant cells to redirect the silencing specificity of a naturally occurring RNAi precursor, which is known to be amplified in its wild-type form (i.e. it produces long-dsRNA), against the pest gene “X” (e.g. TAS gene; which is amplified into long dsRNA and processed into tasiRNAs in its wild-type form). This transcript is marked in FIG. 2 as “Amplified GEiGS precursor”. According to some embodiments, an RNAi precursor which can be used with Model 2 is an RNAi precursor which forms long-dsRNA and is processed to secondary small RNAs, such as, but not limited to, a precursor processed to a trans-acting siRNA (tasiRNA) or a phased small interfering RNA (phasiRNA). Gene Editing induced Gene Silencing (GEiGS) is performed on the gene encoding the RNAi precursor, by using an endonuclease (e.g. CAS9) to induce a double strand break in the gene and providing a DNA “GEiGS oligonucleotide” which introduces into the gene the nucleotide changes required for specificity-redirection through use of Homology Dependent Recombination (HDR). Thus, depending on the “GEiGS oligonucleotide” that is used, the specificity of a portion of the RNAi precursor (e.g. tTAS) will be changed to target pest gene X. The redirected RNAi precursor will be processed by the cellular Dicer into secondary small RNAs (e.g. tasiRNAs) which will also match the pest gene X. In the example depicted in FIG. 2, only one of the tasiRNAs will be altered, resulting in a TAS which is processed to both the wild-type and altered tasiRNAs.

3. A wild type amplifier small RNA forms a RISC complex that is associated with RdRp (the amplifying enzyme).

4. The RdRp synthesizes a complementary antisense RNA strand to the transcript of the amplified GEiGS precursor, forming long-dsRNA.

5. The amplified GEiGS dsRNA is at least partly processed into secondary sRNAs in the plant cell by dicer(s) or other nucleases. Out of these secondary sRNAs, the silencing specificity of the secondary small RNA that corresponds in location to where GEiGS has taken place is towards pest gene X.

6. least part of the non-processed GEiGS long dsRNA is taken up by pests, possibly being processed in the pest to small RNAs, as described above.

7. Possibly, secondary sRNAs which have already been generated within the plant cells (e.g. tasiRNAs in the case of TAS precursor) are taken up as well by the pest, and silence the target gene “X”

Tables 1A and 1B below provide exemplary pest genes which may be targeted by the present methods, and in particular Model 1. Table 2 below provides exemplary pest genes which may be targeted by the present methods, and in particular Model 2. Table 2 also provides suggested RNAi precursors to be targeted by GEiGS (denoted “Backbone”), such as TAS RNA precursors. Table 2 provides suggested small interfering RNAs (denoted “Desired siRNA”), which may be introduced to the suggested backbone using GEiGS, thus enabling the backbone to be processed into these siRNAs in the pests, effecting silencing of the target genes.

TABLE 1A
List of potemial pest-target genes and their accession numbers (including plant homologous genes, as per model 1)

| | | ncbi_ | | Plant | Plant gene | Model-1_ |
|---|---|---|---|---|---|---|
| Pest_ | Pest_ | accesson_ | description_ | host_ | homolog_ | Plant gene homolog_ |
| class | organism | pest gene | Pest gene | organism | accession | description |
| nematode | Heterodera | AF469058.1 | Heterodera glycines | | | |
| | glycines | | cellulose binding | | | |
| | | | protein | | | |
| nematode | Heterodera | AF469060.1 | Heterodera glycines | Aa thaliana | NM_001203752.2 | Arabidopsis thaliana |
| | glycines | | ubiquitin extension | | | ubiquitin 11 |
| | | | protein | | | (UBQ11) |
| nematode | Heterodera | AF500024.1 | Heterodera glycines | Aa thaliana | NM_116351.7 | Arabidopsis thaliana glycosyl |
| | glycines | | putative gland | | | transferase family 1 protein |
| | | | protein G8H07 | | | (AT4G01210) |
| nematode | Heterodera | AF502391.1 | Heterodera glycines | Aa thaliana | NM_4001037071.1 | Arabidopsis thaliana bZIP |
| | glycines | | putative gland | | | transcription factor family |
| | | | protein G10A06 | | | protein (TGA1) |
| nematode | Caenorhabditis | C52E4.1.1 | Caenorhabditis | | | |
| | elegans | | elegans Cysteine | | | |
| | | | Protease related | | | |
| nematode | Meloidogyne | KF734590.1 | Meloidogyne | | | |
| | chitwoodi | | chitwoodi parasitism | | | |
| | | | protein 16D10L | | | |
| | | | (16D10L) | | | |
| whitefly | Bemisia | KF377800.1 | Bemisia tabaci | | | |
| | tabaci | | aquaporin (aqp1) | | | |
| whitefly | Bemisia | KF377802.1 | Bemisia tabaci | | | |
| | tabaci | | nicotinic acetylcholine | | | |
| | | | receptor subunit alpha | | | |
| | | | (nAChRa) | | | |
| whitefly | Bemisia | K1377803.1 | Bemisia tabaci alpha- | | | |
| | tabaci | | 1 glucosidase | | | |
| whitefly | Bemisia | KF377804.1 | Bemisia tabaci heat | | | |
| | tabaci | | shock protein-70 | | | |
| | | | (hsp -70) | | | |
| whitefly | Bemisia | KF442965.1 | Bemisia tabaci | | | |
| | tabaci | | trehalase | | | |
| whitefly | Bemisia | KF442966.1 | Bemisia tabaci | | | |
| | tabaci | | facilitated trehalose | | | |
| | | | transporter-4 | | | |

TABLE 1B
List of potential pest-target genes and their accession numbers

| Pest_class | Pest_organism | ncbi_accession_pest gene | description_Pest gene | Plant host_organism |
|---|---|---|---|---|
| Coleoptera | western corn rootworm (Diabrotica | KR024028.1 | vacuolar ATPase | Corn |
| | virgifera virgifera) | | A subunit | |
| Coleopters | western corn rootworm (Diabrotica | Based on | Snf7 | Corn |
| | virgifera virgifera) | KX982003.1 | | |
| Helicoverpa | cotton bollworm (Helicoverpa armigera) | KR095600.1 | cytochrome P450 | Cotton |
| | | | monooxygenase | |
| | | | (CYP6AE14) | |
| Helicoverpa | cotton bollworm (Helicoverpa armigera) | AY058242 | glulathione-S- | Cotton |
| | | | transferase (GST) | |
| Diptera | Anopholes gambiae | | Chitin synthase 2 | |
| Coleoptera | Diabrotica virgifera virgifera | | Snf 7 | |
| Hemiptera | Acyrthosiphon pisum (pea aphid) | NM_001145904.1 | Aquaporin | Legumes |
| Hemiptera | Acyrthosiphon pisum (pea aphid) | XM_001946489 | V-ATPase E | Legumes |
| Lepidoptera | Chilo infuscatellus (yellow top borer) | JN835468.1 | CiHR3 moulting | Sugarcane and other |
| | | | factor | Poaceae |
| Lepidoptera | Plutella xylostella (diamondback moth) | AY061975.1 | AchE | Cabbage and other |
| | | | (Acetylcholinesterase) | cruciferous crops |
| Lepidoptera | Plutella xylostella (diamondback moth) | KX844829 | CYP6BG1 | Cabbage and other |
| | | | (cytochrome P450) | cruciferous crops |
| Lepidoptera | Spodoptera exigua (Beet armyworm) | DQ062153.1 | Chitin synthase A | Beet and many others |
| Lepidoptera | Spodoptera exigua (Beet army worm) | HQ829425.1 | Beta1 integrin | Beet and many others |
| | | | subunit | |

TABLE 2
Potential pest target genes and examples of their tasiRNA based silencing using GEiGS-(per model 2)

| | | | | Backbone |
|---|---|---|---|---|
| Desired siRNA | SEQ ID NO: | Target organism | Target Gene | Accession # |
| CACAGTAAAATTGAACAAATA | 13 | Heterodera glycines | AF_4058.1 | ATTAS1A |
| | | | | AT2G27400 |
| CACAGTAAAATTGAACAAATA | 14 | Heterodera glycines | AF_469058.1 | ATTAS1C |
| | | | | AT2G39675 |
| CACAGTAAAATTGAACAAATA | 15 | Heterodera glvcines | AF_469058.1 | ATTAS3A |
| | | | | AT3G17185 |
| CACAGTAAAATTGAACAAATA | 16 | Heterodera glycines | AF_469058.1 | ATTAS3C |
| | | | | AT5G57735 |
| CACAGTAAAATTGAACAAATA | 17 | Heterodera glycines | AF_469058.1 | ATTAS3B |
| | | | | AT5G49615 |
| CTGCGATGGCATGCAAATTTT | 18 | Heterodera glycines | AF_469060.1 | ATTAS1A |
| | | | | AT2G27400 |
| CTGCGATGGCATGCAAATTTT | 19 | Heterodera glycines | AF_469060.1 | ATTAS1C |
| | | | | AT2G39675 |
| CTGCGATGGCATGCAAATTTT | 20 | Heterodera glycines | AF_469060.1 | ATTAS3A |
| | | | | AT3G17185 |
| CTGCGATGGCATGCAAATTTT | 21 | Heterodera glycines | AF_469060.1 | ATTAS3C |
| | | | | AT5G57735 |
| CTGCGATGGCATGCAAATTTT | 22 | Heterodera glycines | AF_469060.1 | ATTAS3B |
| | | | | AT5G49615 |
| TAAAATGGAAATAGACAATAT | 23 | Heterodera glycines | AF_500024.1 | ATTAS1A |
| | | | | AT2G27400 |
| TAAAATGGAAATAGACAATAT | 24 | Heterodera glycines | AF_500024.1 | ATTAS1C |
| | | | | AT2G39675 |
| TAAAATGGAAATAGACAATAT | 25 | Heterodera glycines | AF_500024.1 | ATTAS3A |
| | | | | AT3G17185 |
| TAAAATGGAAATAGACAATAT | 26 | Heterodera glycines | AF_500024.1 | ATTAS3C |
| | | | | AT5G57735 |
| TAAAATGGAAATAGACAATAT | 27 | Heterodera glycines | AF_500024.1 | ATTAS3B |
| | | | | AT5G49615 |
| GAGAAGGAAAATACACAATTA | 28 | Heterodera glycines | AF_502391.1 | ATTAS1A |
| | | | | AT2G27400 |
| GAGAAGGAAAATACACAATTA | 29 | Heterodera glycines | AF_502391.1 | ATTAS1C |
| | | | | AT2G39675 |
| GAGAAGGAAAATACACAATTA | 30 | Heterodera glycines | AF_502391.1 | ATTAS3A |
| | | | | AT3G17185 |
| GAGAAGGAAAATACACAATTA | 31 | Heterodera glycines | AF_502391.1 | ATTAS3C |
| | | | | AT5G57735 |
| GAGAAGGAAAATACACAATTA | 32 | Heterodera glycines | AF_502391.1 | ATTAS3B |
| | | | | AT5G49615 |
| TAGTTAGGAAATTTCAAATAA | 33 | Caenorhabditis elegans | C52E4.1.1 | ATTAS1A |
| | | | | AT2G27400 |
| TAGTTAGGAAATTTCAAATAA | 34 | Caenorhabditis elegans | C52E4.1.1 | ATTAS1C |
| | | | | AT2G39675 |
| TAGTTAGGAAATTTCAAATAA | 35 | Caenorhabditis elegans | C52E4.1.1 | ATTAS3A |
| | | | | AT3G17185 |
| TAGTTAGGAAATTTCAAATAA | 36 | Caenorhabditis elegans | C52E4.1.1 | ATTAS3C |
| | | | | AT5G57735 |
| TAGTTAGGAAATTTCAAATAA | 37 | Caenorhabditis elegans | C52E4.1.1 | ATTAS3B |
| | | | | AT5G19615 |
| ATGGGAATATATTAAAACTTT | 38 | Meloidogyne chitwoodi parasitism | KF734590.1 | ATTAS1A |
| | | | | AT2G27400 |
| ATGGGAATATATTAAAACTTT | 39 | Meloidogyne chitwoodi parasitism | KF734590.1 | ATTAS1C |
| | | | | AT2G39675 |
| ATGGGAATATATTAAAACTTT | 40 | Meloidogyne chitwoodi parasitism | KF734590.1 | ATTAS3A |
| | | | | AT3G17185 |
| ATGGGAATATATTAAAACTTT | 41 | Meloidogyne chitwoodi parasitism | KF734590.1 | ATTAS3C |
| | | | | AT5G57735 |
| ATGGGAATATATTAAAACTTT | 42 | Meloidogyne chitwoodi parasitism | KF734590.1 | ATTAS3B |
| | | | | AT5G19615 |
| TGGAGCAATCATTCTGAATGA | 43 | Bemisia tabaci | KF377800.1 | SLTAS3 |
| | | | | JX047545 |
| TGGAGCAATCATTCTGAATGA | 44 | Bemisia tabaci | KF377800.1 | SLTAS3(2) |
| | | | | BE459870 |
| CTCACTCCTTTTAAACAAATA | 45 | Bemisia tabaci | KF377802.1 | SLTAS3 |
| | | | | JX047545 |
| CTCACTCCTTTTAAACAAATA | 46 | Bemisia tabaci | KF377802.1 | SLTAS3(2) |
| | | | | BE459870 |
| ATACATATAGATTGATAACAA | 47 | Bemisia tabaci | KF377803.1 | SLTAS3 |
| | | | | JX047545 |
| ATACATATAGATTGATAACAA | 48 | Bemisia tabaci | KF377803.1 | SLTAS3(2) |
| | | | | BE459870 |
| CCAGGATTCCATGTAAAAAAA | 49 | Bemisia tabaci | KF377804.1 | SLTAS3 |
| | | | | JX047545 |
| CCAGGATTCCATGTAAAAAAA | 50 | Bemisia tabaci | KF377804.1 | SLTAS3(2) |
| | | | | BE459870 |
| CAACCGCATGATAAACGTGAA | 51 | Bemisia tabaci | KF442965.1 | SLTAS3 |
| | | | | JX047545 |
| CAACCGCATGATAAACGIGAA | 52 | Bemisia tabici | KF442965.1 | SLTAS3(2) |
| | | | | BE459870 |
| CTGCATGTTCTTCATCCCCGA | 53 | Bemisia tabaci | KF442966.1 | SLTAS3 |
| | | | | JX047545 |
| CTGCATGTTCTTCATCCCCGA | 54 | Bemisia tabaci | KF442966.1 | SLTAS3(2) |
| | | | | BE459870 |

Arabidopsis and Tomato Bombardment and Plant Regeneration

Arabidopsis Root Preparation

Chlorine gas sterilized Arabidopsis (cv. Col-0) seeds are sown on MS minus sucrose plates and vernalised for three days in the dark at 4° C., followed by germination vertically at 25° C. in constant light. After two weeks, roots are excised into 1 cm root segments and placed on Callus Induction Media (CIM: 1/2 MS with B5 vitamins, 2% glucose, pH 5.7, 0.8% agar, 2 mg/l IAA, 0.5 mg/l 2,4-D, 0.05 mg/l kinetin) plates. Following six days incubation in the dark, at 25° C., the root segments are transferred onto filter paper discs and placed onto CIMM plates, (1/2 MS without vitamins, 2% glucose, 0.4 M mannitol, pH 5.7 and 0.8% agar) for 4-6 hours, in preparation for bombardment.

Tomato Explant Preparation:

Tomato seeds are surface sterilized with commercial bleach for 20 minutes, followed by washing with sterile water 3 times in sterile conditions. The seeds are cultured on germination media (MS+ vitamins, 0.6% agarose, pH=5.8) and placed in 25° C. with 16/8 hours light/dark cycles.

Cotyledons are cut from 8 days old tomato plants, to approximately 1 cm² and placed on pre-bombardment culture (MS+ vitamins, 3% sucrose, 0.6% agarose, pH=5.8, 1 mg/l BAP, 0.2 mg/l IAA) for 2 days in the dark in 25° C. Then, explants are transferred to the center of a target plate (containing MS+ vitamins, 3% Mannitol, 0.6% agarose, pH=5.8) for 4 hours.

Bombardment

Plasmid constructs are introduced into the root tissue via the PDS-1000/He Particle Delivery (Bio-Rad; PDS-1000/He System #1652257), several preparative steps, outlined below, are required for this procedure to be carried out.

Gold Stock Preparation

40 mg of 0.6 μm gold (Bio-Rad; Cat: 1652262) is mixed with 1 ml of 100% ethanol, pulse centrifuged to pellet and the ethanol is removed. This wash procedure is repeated two more times.

Once washed, the pellet is resuspended in 1 ml of sterile distilled water and dispensed into 1.5 ml tubes of 50 μl aliquot working volumes.

Bead Preparation

In short, the following is performed:

Typically, a single tube is sufficient gold to bombard 2 plates of Arabidopsis roots, (2 shots per plate), therefore each tube is distributed between 4 (1,100 psi) Biolistic Rupture disks (Bio-Rad).

Bombardments requiring multiple plates of the same sample, tubes are combined and volumes of DNA and CaCl₂/spermidine mixture adjusted accordingly, in order to maintain sample consistency and minimize overall preparations.

The following protocol summarizes the process of preparing one tube of gold, these should be adjusted according to number of tubes of gold used.

All subsequent processes are carried out at 4° C. in an Eppendorf thermomixer. Plasmid DNA samples are prepared, each tube comprising 11 μg of DNA added at a concentration of 1000 ng/μl

1) 493 μl ddH2O is added to 1 aliquot (7 μl) of spermidine (Sigma-Aldrich), giving a final concentration of 0.1 M spermidine. 1250 μl 2.5M CaCl2 is added to the spermidine mixture, vortexed and placed on ice.

2) A tube of pre-prepared gold is placed into the thermomixer, and rotated at a speed of 1400 rpm.

3) 11 μl of DNA is added to the tube, vortexed, and placed back into the rotating thermomixer.

4) To bind, DNA/gold particles, 70 μl of spermidine CaCl₂ mixture is added to each tube (in the thermomixer).

5) The tubes are vigorously vortexed for 15-30 seconds and placed on ice for about 70-80 seconds.

6) The mixture is centrifuged for 1 minute at 7000 rpm, the supernatant is removed and placed on ice.

7) 500 μl 100% ethanol is added to each tube and the pellet is resuspended by pipetting and vortexed.

8) The tubes are centrifuged at 7000 rpm for 1 minute.

9) The supernatant is removed and the pellet resuspended in 50 μl 100% ethanol, and stored on ice.

Macro Carrier Preparation

The following is performed in a laminar flow cabinet:

1) Macro carriers (Bio-Rad), stopping screens (Bio-Rad), and macro carrier disk holders are sterilized and dried.

2) Macro carriers are placed flatly into the macro carrier disk holders.

3) DNA coated gold mixture is vortexed and spread (5 μl) onto the center of each Biolistic Rupture disk.

Ethanol is allowed to evaporate.

PDS-1000 (Helium Particle Delivery System)

In short, the following is performed:

The regulator valve of the helium bottle is adjusted to at least 1300 psi incoming pressure. Vacuum is created by pressing vac/vent/hold switch and holding the fire switch for 3 seconds. This ensured helium is bled into the pipework.

1100 psi rupture disks are placed into isopropanol and mixed to remove static.

1) One rupture disk is placed into the disk retaining cap.

2) Microcarrier launch assembly is constructed (with a stopping screen and a gold containing microcarrier).

3) Petri dish Arabidopsis root callus is placed 6 cm below the launch assembly.

4) Vacuum pressure is set to 27 inches of Hg (mercury) and helium valve is opened (at approximately 1100 psi).

5) Vacuum is released; microcarrier launch assembly and the rupture disk retaining cap are removed.

6) Bombardment on the same tissue (i.e. each plate is bombarded 2 times).

7) Bombarded roots are subsequently placed on CIM plates, in the dark, at 25° C., for additional 24 hours.

Co-Bombardments

When bombarding GEiGS plasmids combinations, 5 μg (1000 ng/μl) of the sgRNA plasmid is mixed with 8.5 μg (1000 ng/μl) swap plasmid (e.g. DONOR) and 11 μl of this mixture is added to the sample. If bombarding with more GEiGS plasmids at the same time, the concentration ratio of sgRNA plasmids to swap plasmids (e.g. DONOR) used is 1:1.7 and 11 μg (1000 ng/μl) of this mixture is added to the sample. If co-bombarding with plasmids not associated with GEiGS swapping, equal ratios are mixed and 11 μg (1000 ng/μl) of the mixture is added to each sample.

Transfection of Col-0 Protoplasts

Arabidopsis thaliana (Col-0) protoplasts were transfected with vectors coding for Crispr/Cas9 and a donor template to achieve HDR-mediated swaps. The experiment was designed such that sequences in the Tas1b (AtTAS1b_AT1G50055) or Tas3a (AtTAS3a_AT3G17185) genes were swapped, generating sRNAs that target 30 bp sequences in the above-described nematode target genes. Without wishing to be bound by theory or mechanism, the rationale in generating a long dsRNA, which targets 30 bp sequences in the nematode is to ensure that when the dsRNA. is processed in the nematode to secondary silencing RNAs it creates functional silencing RNA molecules even if the length of secondary silencing RNAs formed in the nematode is different than that formed in the plant.

Two swaps were designed in the TAS1b locus, and two swaps in the TAS3a locus. Swaps are independent from each other. The DONOR template (1 kb) were synthesised in plasmids (synthesised by Twist, USA).

The protoplast concentration was determined using a hemocytometer and viability using Trypan Blue (approx.: 30 μl protoplasts, 65 μl mmg, 5 μl Trypan blue). The protoplasts were dilute or concentrated protoplasts to a final density of 2×10⁶ cells/ml.

For PEG transfection, the molar ratio of sgRNA Vector (Crispr/Cas9, sgRNA, mCHERRY): DONOR vector was 1:20, which translates into 3.9 μg sgRNA Vector and approximately 21.61 μg DONOR Vector per transfection. To 1 ml of protoplasts, 1 ml of PEG solution was added slowly. PEG solution was made fresh (2 g PEG 4000 (Sigma) per 5 ml, 0.2M mannitol, 0.5 ml of 1M CaCl2). Tubes were incubated in the dark at room temperature for 20 min, then 4 ml of W5 was added and tubes were mixed by inverting. Protoplast centrifugated pellet was then resuspended in 5 ml. PCA (Protoplast regeneration media) to allow the cells to divide, favouring HDR.

Cell Analysis

24-72 hours after plasmid delivery, cells are collected and resuspended in D-PBS media. Half of the solution is used for analysis of luciferase activity, and half is analyzed for small RNA sequencing. Analysis of Dual luciferase assay is carried out using Dual-Glo® Luciferase Assay System (Promega, USA) according to the manufacturer's instructions. Total RNA is extracted with Total RNA Purification Kit (Norgene Biotek Corp., Canada), according to manufacturer's instructions. Small RNA sequencing is carried out for the identification of the desired mature small RNA in these samples.

Arabidopsis Plant Regeneration

For shoot regeneration, a modified protocol from Valvekens et al. [Valvekens, D. et al., Proc Natl Acad Sci USA (1988) 85(15): 5536-5540] is carried out. Bombarded roots are placed on Shoot Induction Media (SIM) plates, which included 1/2 MS with B5 vitamins, 2% glucose, pH 5.7, 0.8% agar, 5 mg/l 2 iP, 0.15 mg/l IAA. Plates are left in 16 hours light at 25° C.-8 hours dark at 23° C. cycles. After 10 days, plates are transferred to MS plates with 3% sucrose, 0.8% agar for a week, then transferred to fresh similar plates. Once plants regenerated, they are excised from the roots and placed on MS plates with 3% sucrose, 0.8% agar, until analyzed.

Tomato Post-Bombardment Culture and Plant Regeneration

Bombarded explants are placed in the dark at 25° C. on MS media (MS+ vitamins, 3% sucrose, 0.4% agargel, pH=5.8, 1 mg/l BAP, 0.2 mg/l IAA) for two days. Explants are transferred to 16/8 light/dark cycles, and sub cultured every 2 weeks. Regenerating shoots are transferred to root induction media (MS+ vitamins, 3% sucrose, 2.25% gelrite, pH=5.8, 2 mg/l IBA).

Rooting plants are washed in water, to take all agar residues, put in soil and covered. After a week of acclimatization, lid is gradually taken off and plants are hardened.

Genotyping

Tissue samples are treated, and amplicons amplified in accordance with the manufacturer's recommendations using Phire Plant Direct PCR Kit (Thermo Scientific). Oligos used for these amplifications are designed to amplify the genomic region spanning from a region in the modified sequence of the GEiGS system, to outside of the region used as HDR template, to distinguish from DNA incorporation. Different modifications in the modified loci are identified through different digestion patterns of the amplicons, given by specifically chosen restriction enzymes.

Genomic PCR Reactions

Cell samples (A, B, C, D, E, as discussed in Example 3, below) were processed for genomic DNA using a RNA/DNA Purification Kit (Norgen) according to the manufacturer's instructions. Samples were quantified by Qubit and DNA was stored at −20° C.

An unspecific primer flanking the swap region was used for the Tas1b (AtTAS1b_AT1G50055) and Tas3a (AtTAS3a_AT3G17185) sequences. As a negative control the same swap specific reactions were carried out using wild-type (WT) DNA as template. As a positive PCR control a specific PCR for WT DNA was carried out for all samples. Q5® High-Fidelity 2X Master Mix was used for PCR amplifications.

5 μl of each PCR reaction were run on 0.8% agarose gels. Band sizes were estimated by comparison to a molecular weight marker (MW): 1 kb Plus DNA Ladder (NEB).

To confirm swaps, a Nested PCR reaction was carried out. The first genomic PCR comprised unspecific forward and reverse primers flanking the HDR region. PCR products were diluted 1/100 with mili-q ultrapure water and then the aforementioned specific swap PCRs were carried out. Unspecific primers used for the first PCR in the Nested approach have annealing sites flanking the annealing sited for the nested primers.

Primers Used:

Unspecific primer for Tas1b:
Tas1b_WT_Nested_Non_Speeific_DNA_R:
(SEQ ID NO: 63)
5′-accaatttgacccaaaaaggc-3′
Swap-specific primers for Tas1b:
Tas1b_Splicing30_Nested_DNA_F:
(SEQ ID NO: 64)
5′-GCAGCAGATCAATGAAATTCAACG-3′
Tas1b_Y2530_Nested_DNA_F:
(SEQ ID NO: 65)
5′-agCCGCTCTGTGGATTCTTG-3′
Unspecific primer for Tas3a:
Tas3A_WT_Nested_Non_Specific_DNA_R:
(SEQ ID NO: 66)
5′-aaactcctcgcctcttggtg-3′
Swap-specific primers for Tas3a:
Tas3a_Ribo3a30_Nested_DNA_F:
(SEQ ID NO: 67)
5′-TCTTCAGCACCTTCACCTTACG-3′
Tas3a_Spliceo30_Nested_DNA_F:
(SEQ ID NO: 68)
5′-TCCTTTTTGACCAACATTTGTTTGT-3′

Positive Control Reactions

WT Tas1b Specific:
Tas1b_WT_Nested_Non_Specific_DNA_R:
(SEQ ID NO: 69)
5′-accaattttacccaaaaaggc-3′
Tas1b_WT_Nested_DNA_F:
(SEQ ID NO: 70)
5′-tggacttagaatatgctatgttggac-3′
WT Tas3a Specific:
Tas3a_WT_Nested_Non_Specific_DNA_R
(SEQ ID NO: 71)
5′-aaactcctcgcctcttggtg-3′
Tas3a_WT_Nested_DNA_F
(SEQ ID NO: 72)
5′-tctatctctacctctaattcgttcgag-3′

DNA and RNA Isolation

Samples are harvested into liquid nitrogen and stored in −80° C. until processed. Grinding of tissue is carried out in tubes placed in dry ice, using plastic Tissue Grinder Pestles (Axygen, US). Isolation of DNA and total RNA from ground tissue is carried out using RNA/DNA Purification kit (Norgen Biotek Corp., Canada), according to manufacturer's instructions. In the case of low 260/230 ratio (<1.6), of the RNA fraction, isolated RNA is precipitated overnight in −20° C., with 1 μl glycogen (Invitrogen, US) 10% V/V sodium acetate, 3 M pH 5.5 (Invitrogen, US) and 3 times the volume of ethanol. The solution is centrifuged for 30 minutes in maximum speed, at 4° C. This is followed by two washes with 70% ethanol, air-drying for 15 minutes and resuspending in Nuclease-free water (Invitrogen, US).

RNA Extraction

Cell samples (A, B, C, D, E, as discussed in Example 3, below) were processed for RNA purification using a RNA/DNA Purification Kit (Norgen) according to the manufacturer's instructions. Samples were quantified by Qubit. RNA was stored at −80° C.

DNAse Treatment of RNA Samples

An RT-PCR reaction followed by PCR was used to look specifically for small dsRNA fragments containing the swaps (<200 bp), in order to prove the biogenesis of dsRNA which is capable of targeting nematode target genes. To do so, the Turbo DNA-Free Kit (Invitrogen) was used according to the manufacturer's instructions. A DNAse treatment was further performed and the concentration of samples was normalised.

Reverse Transcription (RT) and Quantitative Real-Time PCR (qRT-PCR)

One microgram of isolated total RNA is treated with DNase I according to manufacturer's manual (AMPD1; Sigma-Aldrich, US). The sample is reverse transcribed, following the instructor's manual of High-Capacity cDNA Reverse Transcription Kit (Applied Biosystems, US).

For gene expression, Quantitative Real Time PCR (qRT-PCR) analysis is carried out on CFX96 Touch™ Real-Time PCR Detection System (BioRad, US) and SYBR® Green JumpStart™ Taq ReadyMix™ (Sigma-Aldrich, US), according to manufacturer's' protocols, and analyzed with Bio-RadCFX manager program (version 3.1).

RT-PCR of RNA Samples for Expression Analysis for Tas1b and Tas3a Swaps in Col-0 Cells

For RT-PCR, cDNA was generated using unspecific primers for Tas1b and Tas3a, by the qScript Flex cDNA Synthesis Kit (Quanta BioSciences). One cDNA reaction was done for the sense strand and another for the anti-sense of each of Tas1b and Tas3a. Samples to treat contained 165 ng/μl RNA.

A negative control was used with no Reverse Transcriptase (−RT control) for all RT-PCR reactions (same treatment but with H₂O instead of Reverse Transcriptase). This was to make sure amplification in downstream PCR reactions was not happening because of DNA carry-over. A water negative control was performed for each PCR reaction. A master mix was made with RNA for +RT/−RT for each treatment. Additional Master mixes were made—(i) with Reverse Transcriptase and Buffer (+RT) and (ii) water and Buffer (−RT) for all samples. Final primer concentration: 1 μM

Primers:

Tas1b

Tas1b Sense:
Tas1b_RT_A_R:
(SEQ ID NO: 93)
5′-TAACATAAAAATATTACAAATATCAITCCG-3′
Tasib Antisense:
Tas1b_RT_B_F:
(SEQ ID NO: 94)
5′-TCAGAGTAGTTATGATTGATAGGATGG-3′

These primers were used for treatments A, B, and E.

Tas3a

Tas3a Sense:
Tas3a_RT_A_R:
(SEQ ID NO: 95)
5′-GCTCAGGAGGGATAGACAAGG-3′
Tas3a Antisense:
Tas3a_RT_B_F:
(SEQ ID NO: 96)
5′-CTCGTTTTACAGATTCTATTCTATCTC-3′

These primers were used for treatments C, D and E.

PCR on cDNA to Detect Expression of Tas1b and Tas3a Redirected Towards Nematode Targets

In order to detect dsRNA transcribed from Tas1b or Tas3a genes which have been redirected to target nematode genes, PCR reactions were carried out using the cDNA as a template with one unspecific primer for Tas3a or Tas1b, and another primer which is Swap specific (i.e. binds only the relevant Tas sequence in which nucleotides have been swapped following GEiGS-mediated redirection). Unspecific primer annealing site was located slightly downstream the sequence used for making cDNA. Specific primer annealing sites were located less than 200 bp dowstream from the unspecific primer annealing site. The approach was the same for analysing expression of both strands of the dsRNA: Sense and Antisene. Reactions were carried out also for −RT cDNA reactions to make sure amplification did not happen from residual DNA in the sample after DNAse treatment. As a negative control each reaction was carried out on WT DNA as well to prove that amplification is Swap specific. A H₂O )negative control was included for each PCR reaction. 5 ul of each cDNA PCR reaction were used as template.

Primers:

Tas3a Sense Strand Specific Reactions:

Ribosomal protein 3a specific:
Tas3a_RNA_Non_Specific_A_F:
(SEQ ID NO: 97)
5′-TGACCTIGTAAGACCCCATCTC-3′
Tas3a_RNA_Ribo3a30_Specific_A_R:
(SEQ ID NO: 98)
5′-AggagaaaATTCGTAAGGTGAAGG-3′
WT Specific:
Tas3a_RNA_Non_Specific_A_F:
(SEQ ID NO: 99)
5′-TGACCTTGTAAGACCCCATCTC-3′
Tas3a_RNA_WT_Specific_A_R:
(SEQ ID NO: 100)
5′-GGTAGGAGAAAATGACTCGAACG-3′

Tas3a Anti-Sense Strand Specific Reactions:

Ribosomal protein 3a specific:
Tas3a_RNA_Non_Specific_B_R:
(SEQ ID NO: 101)
5′-CAACCATACATCAATAACAAACAAAAG-3′
Tas3a_RNA_Ribo3a30_Specific_B_F:
(SEQ ID NO: 102)
5′-ATATAGAATAGATatCGGCTTCTTCAG-3′
WT Specific:
Tas3a_RNA_Non_Specific_B_R:
(SEQ ID NO: 103)
5′-CAACCATACATCAATAACAAACAAAAG-3′
Tas3a_RNA_Spliceo30_Specific_B_F
(SEQ ID NO: 104)
5′-TCCTTTTTGACCAACATTTGTTTGT-3′

Tas1b Sense Strand Specific Reactions:

Y25, beta subunit of COPI complex specific:
Tas1b_RNA_Non_Specific_A_F:
(SEQ ID NO: 105)
5′-GAGTCATTCATCGGTATCTAACC-3′
Tas1b_RNA_Y2530_Specific_A_R:
(SEQ ID NO: 106)
5′-agCCGCTCTGTGGATTCTTG-3′
WT Specific:
Tas1b_RNA_Non_Specific_A_F:
(SEQ ID NO: 107)
5′-GAGTCATTCATCGGTATCTAACC-3′
Tas1b_RNA_WT_Specific_A_R:
(SEQ ID NO: 108)
5′-TGGACTTAGAATATGCTATGTTGGAC-3′

Tas1b Anti-Sense Strand Specific Reactions:

Y25, beta subunit of CON complex specific:
Tas1b_RNA_Non_Specific_B_R:
(SEQ ID NO: 109)
5′-GCATATCCTAAAATATGTTTCGTTAAC-3′
Tas1b_RNA_Y2530_Specific_B_F:
(SEQ ID NO: 110)
5′-TCGCCAAGAATCCACAGAGC-3′;
WT Specific:
Tas1b_RNA_Non_Specific_B_R:
(SEQ ID NO: 111)
5′-GCATATCCTAAAATATGTTTCGTTAAC-3′
Tas1b_RNA_WT_Specific_B_F:
(SEQ ID NO: 112)
5′-TAAGTCCAACATAGCATATTCTAAGTC-3′

Study of Silencing Activity of Long dsRNA in Nicotiana Benthamiana Towards TuMV

Plant Material

Nicotiana benthamiana were grown on soil in long day conditions 16 hours light, 8 hours dark) at 21° C. for 4 weeks until treated.

TuMV-GFP Vector Cloning

TuMW-GFP cDNA cassette was amplified from the vector described in Touriño, A., et al. (Touriño, A., Sánchez, F., Fereres, A. and Ponz, F. (2008). High expression of foreign proteins from a biosafe viral vector derived from Turnip mosaic virus. Spanish Journal of Agricultural Research, 6(S1), p.48). Amplification was done using the primer set 5′-ATGTTTGAACGATCGGGCCCaagggacacgaagtgatccg-3′ (SEQ ID NO: 113) and 5′-CTCCACCATGTTCCCGGGggcacagagtgttcaacccc-3′ (SEQ ID NO: 114). The amplicon was cloned into a binary vector, harbouring the NPTII resistance gene, in the T-DNA region through In-Fusion reaction, according to manufacturer's protocol. For the purpose of agrobacterium infiltration the vector was subsequently transformed into agrobacterium strain GV3101.

Agroinduction and Leaf Infiltration

1. Liquid culture of agrobacterium was grown in LB

2. The cells were spined down and washed once with MMA media (10 mM MES, 10 mM MgCl2, and 200 μM acetosyringone, pH=5.6)

3. The cells were spined down and the sup was taken out. Pellet was resuspended in MMA media to OD600=0.5.

4. Culture was shaken gently in the dark for 6 hours.

5. Cultures were combined as required (1:1 ratios between bacteria containing different vectors, each agrobacterium containing a vector that expresses a single gene). Total final agrobacterium density—OD600=0.5. TuMV-GFP vector was added to a final density of OD600=0. 0001.

6. Leaves of a 4-weeks old N. benthamiana plant were infiltrated with the induced cultures, using a needleless syringe.

Gene Sequences Used for GEiGS-dsRNA Silencing

SEQ ID NO: 115
AtTAS1B (At1g50055)
SEQ ID NO: 116
GEiGS-TuMV
SEQ ID NO: 117
GEiGS-TuMV- mature siRNA
SEQ ID NO: 118
GEiGS-dummy
SEQ ID NO: 119
GEiGS-dummy- mature siRNA
SEQ ID NO: 120
miR173_AT3G23125
SEQ ID NO: 121
miR173-mature miRNA

Study of Arabidopsis Protection from TuMV Infection and Disease

Plant Material

Arabidopsis seeds, collected from plants harboring the desired GEiGS sequence, are chlorine gas sterilized and sown 1 seed/well in MS-S agar plates. Two weeks old seedlings are transferred to soil. Plants are grown in 24° C. under 16 hours light/8 hours dark cycles. Wild type non-modified (plants) are grown and treated in parallel, as control.

Plant Inoculation and Analysis

Procedures for the inoculation and analysis of plants with TuMV vectors are well established in the art and were previously described [Sardaru, P. et al., Molecular Plant Pathology (2018), 19:1984-1994]. Four weeks old Arabidopsis seedlings are inoculated with TuMV as previously described [Sánchez, F. et al. (1998) Virus Research, 55(2): 207-219] or TuMV-GFP as previously described [Touriño, A., et al. (2008) Spanish Journal of Agricultural Research, 6(S1), p.48] expressing viral vectors. Scoring of symptoms, in the case of TuMV, takes place 10-28 days post inoculation. Analysis of GFP signal, in the case of TuMV-GFP, takes place 7-14 days post inoculation.

In addition, 14 days post inoculation, new leaves growing above the inoculation site, are harvested, and total RNA is extracted using Total RNA Purification Kit (Norgene Biotek Corp., Canada), according to manufacturer's instructions. Small RNA analysis and RNA-seq is carried out for profiling of gene expression and small RNA expression on these samples.

Study of Tomato Infection with Whitefly

Plant Material

Tomato plants are grown from seeds collected from plants harboring the desired GEiGS sequence, at one plant per pot in 22° C. under 16 hours light/8 hours dark cycles. Wild type non-modified (plants) are grown and treated in parallel, as control.

Whitefly Inoculation

Five female whiteflies are introduced to a 4 weeks old tomato plant. The whiteflies are placed into a clip cage holding a single leaf. After 5 days, dead and living whiteflies, as well as eggs, are counted.

In addition, 5 days post inoculation, the infected leaf is harvested, and total RNA is extracted. Dead and living whiteflies are collected separately, and total RNA is extracted from them as well. Small RNA analysis and RNA-seq is carried out for profiling of gene expression and small RNA expression on these samples.

Study of dsRNA Targeting a Nematode Gene

Nematode

Plant-parasitic cyst nematodes Globodera rostochiensis (pathotype Ro1, acquired from the James Hutton Institute collection) were maintained at the University of Cambridge under DEFRA licence 125034/359149/3. Nematodes were maintained on Solanum tuberosum cultivar Désirée. Fifty cysts were combined with a 50:50 mix of sand:loam in a 7 inch diameter pot. One tuber was planted per pot, and watered regularly for a period of 3 months at 20° C. Plants were allowed to dry for 1 month, and cysts were collected from the soil using flotation followed by nested sieving. Juveniles were hatched from the cysts by incubation with tomato root diffusate, replaced every 2-3 days for a period of up to 14 days. Hatched juveniles were stored at 4° C. in water containing 0.01% Tween-20 for up to 1 week before being used in subsequent assays.

Sequences Used
AtTAS3a_AT3G17185—SEQ ID NO: 122
GEiGS-Ribosomal protein 3a-transcript—SEQ ID NO: 123
GEiGS-Ribosomal protein 3a-transcript—SEQ ID NO: 124—indicates region of homology to the target gene, generated through the GEiGS design to generate siRNA in nematodes (i.e. the expected processed siRNA)
GEiGS-Spliceosomal SR protein-transcript—SEQ ID NO: 125
GEiGS-Spliceosomal SR protein-transcript—SEQ ID NO: 126—indicates region of homology to the target gene, generated through the GEiGS design to generate siRNA in nematodes(i.e. the expected processed siRNA)
miR390_AT2G38325—SEQ ID NO: 127

RNA Preparation for Feeding

Total RNA from infiltrated N. benthamiana leaves was extracted with Tri-Reagent (Sigma-Aldrich, USA), with two chloroform washes, and overnight precipitation in isopropanol. Recovered RNA was further cleaned with standard sodium acetate precipitation.

All recovered RNA was cleaned using Amicon® Ultra 0.5 mL Centrifugal Filters 3KD cut-off (Merck, USA), according to manufacturer's instructions, and 3 washed with DDW. RNA was quantified using nanodrop.

Nematode Feeding Protocol

RNA was diluted to 1.76 μg/μl in 1×M9 and 50 mM Octopamine. 3500 J2 per repeat were pelleted in 1.5 ml Eppendorf to approx volume of 5 μl. 25 μl of the RNA solution was added to the nematodes and incubated at 20° C. in a heat block and rotation at 300 rpm (final RNA concentration was 1.47 μg/μl). After 72 hours, washes were carried out by spinning down the nematodes (10 k g 1 min), removal of supernatant. Washes were repeated 3 times with 500 μl RNAse free water. Pellet was snap frozen in Liquid nitrogen and kept in −80° C. until treated.

Nematode RNA Extraction and Purification

RNA isolation was carried out using the Direct-zol RNA Miniprep: Zymo Research Cat. No. R2052, as per manufacturer's recommendations.

Using a microtube pestle, the frozen (liq N2 or Dry ice) tissue samples (≤25 mg) were crushed until powdered in eppendorf, and 600 μl TRI Reagent was added to sample and grinding continued until fully homogenised. The following steps were then performed at room temperature and centrifugation at 10,000-16,000×g for 30 seconds, unless specified:

1. An equal volume of ethanol (95-100%) was added to a sample lysed in TRI Reagent or similar1 and mixed thoroughly.

2. The mixture was transfered into a Zymo-Spin™ IICR Column2 in a Collection Tube and centrifuged. The column was transferred into a new collection tube and the flow through discarded.

3. DNaseI treatment was carried out in column

(3a) 400 μl RNA Wash Buffer were added to the column and centrifuged.

(3b) In an RNase-free tube, 5 μl DNase I (6 U/μl) and 75 μl DNA Digestion Buffer were added and mixed. The mix was added directly to the column matrix.

(3c) Incubated at room temperature (20-30° C.) for 15 minutes.

4. 400 μl Direct-zol™ RNA PreWash was added to the column and centrifuged. The flow-through was discarded and step was repeated.

5. 700 μl RNA Wash Buffer was added to the column and centrifuged for 2 minutes to ensure complete removal of the wash buffer. The column was transfered carefully into an RNase-free tube.

6. To elute RNA, 30 μl of DNase/RNase-Free Water was added directly to the column matrix and centrifuged.

7. RNA was quantified using a NanoDrop spectrophotometer/fluorometer or a Qubit fluorometer, RNA was either used immediately or stored frozen at ≤−70° C.

qRT cDNA Library Preparation

(Quanta BIOSCIENCE: qScript Flex cDNA Synthesis Kit)

1. All components (excluding enzyme) were thawed, mixed thoroughly, and centrifuged (before use), and placed on ice (before use).

2. The following were added to a 0.2 mL thin-walled PCR tube or 96-well PCR reaction plate sitting on ice:

3.

| Component | volume | |
|---|---|---|
| RNA (1 μg to 10 μg total RNA) | variable | |
| Nuclease-free water | variable | |
| Oligo dT | 2 | μl |
| Final volume | 15.0 | μl |

Note:
(For a mixed primer strategy, 2 μl of Oligo dT was used. For multiple first-strand reactions, a master mix was prepared with the reaction mix and RT and dispensed 5 μl into each tube).

4. Components were mixed by gentle vortexing and then centrifuged 10s to collect contents

5. Incubated for 5 min at 65° C. and then snaped chill in ice.

6. The following were added to the primed RNA template mixture:

| Component | volume | |
|---|---|---|
| qScript Flex Reaction Mix (5X) | 4 | μl |
| qScript Reverse Transcriptase | 1 | μl |
| final volume | 20.0 | μl |

Note:
(For multiple first-strand reactions, a master mix was prepared with the reaction mix
and RT, and dispensed 5 μl into each tube).

7. Components were mixed by gentle vortexing and then incubated as follows:
60 min at 42° C.
5 min at 85° C.
Hold at 4° C.

8. After completion of cDNA synthesis, an additional 30 μl of dH2O or TE buffer [10 mM Tris (pH 8.0), 0.1 mM EDTA] was added, using 2-3 μl for 20 μl qRTPCR reactions. cDNA could be stored at −20° C.

SYBR Green Jump Start Taq Ready Reaction Protocol

1. All components (except enzyme) were thawed, mixed thoroughly, and centrifuged before use. Kept on ice before use.

2. The following was added to a 0.2 mL thin-walled PCR tube or 96-well PCR reaction plate sitting on ice:

| Component | volume | |
|---|---|---|
| 2X SYBR master mix | 10 | μl |
| Specific Forward primer(10 uM) | 1 | μl |
| Specific Reverse primer(10 uM) | 1 | μl |
| cDNA template | 2-3 | μl |
| Nuclease free dH2O | Variable | |
| final volume | 20 | μl |

3. Samples were incubated as follows:
94° C. 2 min.
94° C. 15 sec.
55-60° C. 60 sec, 35-40 cycles read SYBR signal
Melting curve:
95° C.->65° C. at 20° C. per cycle collectd signal continuously from 65° C.->95° C. per 0.2 sec

Reaction were run in technical triplicates, for both, the gene of interest and the endogenous calibrator.

Primer Sequence

Spliceosomal SR protein:
qRTSpSR_FWD
SEQ ID NO: 128
GCTCAACTGACAAAGAATCTCTCAC
qRTSpSR_REV
SEQ ID NO: 129
TTGAAAATTGGGTCAAAGAAATGCG
Ribosomal protein3a:
qRTRib3a_FWD
SEQ ID NO: 130
GAACGGTCGCTACGATTACGA
qRTRib3a_REV
SEQ ID NO: 131
CAAACGCTCTGTTGAACAGGC
Endogenous gene for normalization:
NEMAACTIN_09251_F
SEQ ID NO: 132
TTCCAGCAGATGTGGATCAG
NEMAACTIN_09251_R
SEQ ID NO: 133
CGGCCTTATTCTTCAAGCAC

Materials for bioinformatic analysis-

Small-RNA raw data in FASTQ format was processed using cutadapt 2.8 with parameters “-m 18-u 4-a NNNNTGGAATTCTCGGGTGCCAAGG” (SEQ IS NO: 138) to trim the sequencing adapter, remove the random adapters, and keep only reads longer than 18 nt. RNA-set raw data in FASTQ format was processed using cutadapt 2.8 with parameters “-m 18-a AGATCGGAAGAGCACACGTCTGAACTCCAGTCA -A AGATCGGAAGAGCGTCGTGTAGGGAAAGAGTGT” (SEQ IS NO: 139) to remove sequencing adapters and keep reads longer than 18 nt.

An alignment index was created for a pseudo-genome composed by the target sequences using STAR version 2.7.1a with parameter “—genomeSAindexNbases 3” to accomodate the small pseudo-genome.

Small-RNA adapter-trimmed reads were aligned to the pseudo-genome using STAR 2.7.1a with parameters “—outSAMtype BAM Unsorted—outFilterMismatchNmax 0—alignIntronMax 1—alignEndsType EndToEnd—scoreDelOpen—10000—scoreInsOpen—10000”. RNA-seq adapter-trimmed reads where aligned using the same resources with parameters “—outSAMtype BAM Unsorted—alignEndsType EndToEnd—alignIntronMax 500”,

A custom python script was used to filter aligned small-RNA reads to lengths between 20 and 24 nucleotides, and RNA-seq reads to lengths greater than 50 nucleotides.

Read coverage against the target sequences was calculated using bedtools 2.29.2 with parameters “genomecov—bg—scale {factor}” where the factor was calculated to normalise read counts to reads per million (RPM).

Coverage plots were generated using the Sushi package for R, version 1.25.0.

### Example 1A

### Genome Editing Induced Gene Silencing (GEiGS)

In order to design GEiGS oligos, template non-coding RNA molecules (precursors) that are processed and give raise to derivate small silencing RNA molecules (matures) are required. Two sources of precursors and their corresponding mature sequences were used for generating GEiGS oligos. For miRNAs, sequences were obtained from the miRBase database [Kozomara, A. and Griffiths-Jones, S., Nucleic Acids Res (2014) 42: D68,ÄíD73]. tasiRNA precursors and matures were obtained from the tasiRNAdb database [Zhang, C. et al, Bioinformatics (2014) 30: 1045,Äí1046].

Silencing targets were chosen in a variety of host organisms (data not shown). siRNAs were designed against these targets using the siRNArules software [Holen, T., RNA (2006) 12: 620,Äí1625.]. Each of these siRNA molecules was used to replace the mature sequences present in each precursor, generating “naive” GEiGS oligos. The structure of these naive sequences was adjusted to approach the structure of the wild type precursor as much as possible using the ViennaRNA Package v2.6 [Lorenz, R. et al., ViennaRNA Package 2.0. Algorithms for Molecular Biology (2011) 6: 26]. After the structure adjustment, the number of sequences and secondary structure changes between the wild type and the modified oligo were calculated. These calculations are essential to identify potentially functional GEiGS oligos that require minimal sequence changes with respect to the wild type.

CRISPR/cas9 small guide RNAs (sgRNAs) against the wild type precursors were generated using the CasOT software [Xiao, A. et al., Bioinformatics (2014) 30: 1180, Äí11.82]. sgRNAs were selected where the modifications applied to generate the GEiGS oligo affect the PAM region of the sgRNA, rendering it ineffective against the modified oligo.

### Example 1B

### Gene Silencing of Endogenous Plant Gene—PDS

In order to establish a high-throughput screening for quantitative evaluation of endogenous gene silencing using Genome Editing Induced Gene Silencing (GEiGS), the present inventors considered several potential visual markers. The present inventors chose to focus on genes involved in pigment accumulation, such as those encoding for phytoene desaturase (PDS). Silencing of PDS causes photobleaching (FIG. 8B) which allows to use it as robust seedling screening after gene editing as proof-of-concept (POC). FIGS. 8A-C show a representative experiment with N. benthamiana and Arabidopsis plants silenced for PDS. Plants show the characteristic photobleaching phenotype observed in plants with diminished amounts of carotenoids.

In the POC experiment, choosing siRNAs was carried out as follows:

In order to initiate the RNAi machinery in Arabidopsis or Nicotiana benthamiana against the PDS gene using GEiGS application, there is a need to identify effective 21-24 bp siRNA targeting PDS. Two approaches are used in order to find active siRNA sequences: 1) screening the literature—since PDS silencing is a well-known assay in many plants, the present inventors are identifying well characterized short siRNA sequences in different plants that might be 100% match to the gene in Arabidopsis or Nicotiana benthamiana. 2) There are many public algorithms that are being used to predict which siRNA will be effective in initiating gene silencing to a given gene. Since the predictions of these algorithms are not 100%, the present inventors are using only sequences that are the outcome of at least two different algorithms.

In order to use siRNA sequences that silence the PDS gene, the present inventors are swapping them with a known endogenous non-coding RNA gene sequence using the CRISPR/Cas9 system (e.g. changing a miRNA sequence, changing a long dsRNA sequence, creating antisense RNA, changing tRNA etc.). There are many databases of characterized non-coding RNAs e.g. miRNAs; the present inventors are choosing several known Arabidopsis or Nicotiana benthamiana endogenous non-coding RNAs e.g. miRNAs with different expression profiles (e.g. low constitutive expression, highly expressed, induced in stress etc.). For example, in order to swap the endogenous miRNA sequence with siRNA targeting PDS gene, the present inventors are using the RR approach (Homologous Recombination). Using HR, two options are contemplated: using a donor ssDNA oligo sequence of around 250-500 nt which includes, for example, the modified miRNA sequence in the middle or using plasmids carrying 1 Kb-4 Kb insert which comprises only minimal changes with respect to the miRNA surrounding in the plant genome except the 2×21 bp of the miRNA. and the *miRNA that is changed to the siRNA of the PDS (500-2000 bp up and downstream the siRNA, as illustrated in FIG. 7). The transfection includes the following constructs: CRISPR:Cas9/GFP sensor to track and enrich for positive transformed cells, gRNAs that guides the Cas9 to produce a double stranded break (DSB) which is repaired by HR depending on the insertion vector/oligo. The insertion vector/oligo contains two continuous regions of homology surrounding the targeted locus that are replaced (i.e. miRNA) and is modified to carry the mutation of interest (i.e. siRNA). If plasmid is used, the targeting construct comprises or is free from restriction enzymes-recognition sites and is used as a template for homologous recombination ending with the replacement of the miRNA with the siRNA of choice. After transfection to protoplasts, FACS is used to enrich for Cas9/sgRNA-transfected events, protoplasts are regenerated to plants and bleached seedlings are screened and scored (see FIG. 5). As control, protoplasts are transfected with an oligo carrying a random non-PDS targeting sequence. The positive edited plants are expected to produce siRNA sequences targeting PDS and therefore PDS gene is silenced and seedling are seen as white compared to the control with no gRNA. It is important to note that after the swap, the edited miRNA will still be processed as miRNA because the original base-pairing profile is kept. However, the newly edited processed miRNA has a high complementary to the target (e.g. 100%), and therefore, in practice, the newly edited small RNA will act as siRNA.

### Example 1C

### Harboring Resistance of Arabidopsis Plants to TuMV Viral Infection

Changes in the Arabidopsis genome are designed to introduce silencing specificity in dysfunctional non-coding RNAs to target the Turnip Mosaic Virus (TuMV). These sequences, together with extended homologous arms in the context of the genomic loci, are introduced in PUC57 vector, named DONOR. Guide RNAs are introduced in the CRISPR/CAS9 vector system, in order to generate a DNA cleavage in the desired loci. The CRISPR/CAS9 vector system is co-introduced to the plants with the DONOR vectors via gene bombardment protocol, to introduce desired modifications through Homologous DNA Repair (HDR),

Arabidopsis seedlings with the desired changes in their genome are identified through genotyping, and inoculated with agrobacterium harboring either TuMV or TuMV-CGP and scored for viral response.

### Example 2

### Harboring Resistance of Tomato Plants to Whitefly Infestation

Changes in the tomato genome are designed, to generate non-coding RNAs, according to the GEiGS 2.0 pipeline (discussed above in the ‘General Materials and Experimental Prosedures’ section above), to target the essential gene in whitefly. These sequences, together with extended homologous arms in the context of the genomic loci, are introduced in PUC57 vector, named DONOR. Guide RNAs are introduced in the CRISPR/CAS9 vector system, in order to generate a DNA cleavage in the desired loci. These are co-introduced to the plants with the DONOR vectors via gene bombardment protocol, to introduce desired modifications through Homologous DNA Repair (HDR). Tomato plants, identified to harbor the desired genomic changes through genotyping, are introduced with whiteflies and scored for response.

### Example 3

Using GEiGS on Trans Activating Silencing RNA in A. Thaliana Protoplasts

In order to demonstrate Homology Dependant Recombination (HDR) events in plant cells when using GEiGS to redirect the silencing specificity of tasiRNA, a transfection assay in Arabidopsis protoplasts was carried out using vectors expressing the CRISPR/CAS9 endonuclease, an sgRNA to direct a DNA break, and a “Donor” sequence (also referred to as the GEiGS Donor), to introduce the desired nucleotide changes via GEiGS (also referred to herein as “swaps”). The Donor sequence included a sequence corresponding to the target sequence with the desired nucleotides changes, flanked by homologous arms (about 500 base pairs upstream and downstream of the changed sequences), to facilitate the HDR.

GEiGS approach was essentially according to the principles described above and in WO 2019/058255 (incorporated herein by reference), and as exemplified herein below. Briefly, when a vector comprising the GEiGS-donor is introduced to a cell together with an endonuclease such as Cas9 and an sgRNA targeting the gene to be edited, the GEiGS-oligo sequence is introduced into the genome of the cell (mediated by HDR), such that the edited gene now includes the desired changes (e.g. encodes a TAS gene which can be transcribed to a long dsRNA whose silencing activity has been redirected towards a target of choice).

Two genes were used as backbones for this manipulation, both encoding trans-acting-siRNA-producing (TAS) molecules—TAS1b and TAS3a (see below). The changes to be introduced using GEiGS were chosen such that they would give rise to long dsRNA. and small secondary tasiRNA that would target and silence essential genes in the nematode Globodera rostochiensis. These target genes were chosen based on previous publications that discussed negative effects in a nematode when the genes were targeted using an RNAi technology (Table 3, below), Since these genes were identified in a different strain of nematodes, their homologues were identified through a BLAST search in the Globodera rostochiensis publicly available database (www(dot)parasite(dot)wormbase(dot)org/Globodera_rostochiensis_prjeb13504/Info/Index/), using the chosen genes as queries.

TABLE 3
Target genes in nematodes

| | Target | | | | | | Homologue in |
|---|---|---|---|---|---|---|---|
| | gene | Gene | | Host | | | Globodera |
| | Nematode | symbol | species | plant | Phenotype | Reference | rostochiensis |
| 1 | Splicing | AW828516 | M. | Tobacco | >90% reduction | Yadav et al., | GROS_g05960 |
| | factor | | incognita | | in number of | 2006 | (SEQ ID NO: 55) |
| | | | | | established | | |
| | | | | | nematodes | | |
| 2 | Ribosomal | CB379877 | H. | Soybean | 87% reduction | Klink et at., | GROS_g04462 |
| | protein 3a | | glycines | | in number of | 2009 | (SEQ ID NO: 56) |
| | | | | | female cysts | | |
| 3 | Sliceosomal | BI451523 | H. | Soybean | 88% reduction | Klink et at., | GROS_g04863 |
| | SR | | glycines | | in number of | 2009 | (SEQ ID NO: 57) |
| | protein | | | | female cysts | | |
| 4 | Y25, beta | CB824330 | H. | Soybean | 81% reduction in | Li et al., | GROS_g00263 |
| | subunit of | | glycines | | number of | 2010 a,b | (SEQ ID NO: 58) |
| | COPI | | | | nematode eggs | | |
| | complex | | | | | | |

SiRNA target sites chosen in the gene sequences are depicted in the below sequences:

(SEQ ID NO: 59)
GROS_g05960: TGGAGCAGCAGATCAATGAAATTCAACGAC
(SEQ ID NO: 60)
GROS_g04462: ATTCGTAAGGTGAAGGTGCTGAAGAAGCCG
(SEQ ID NO: 61)
GROS_g04863: AAAAACAAACAAATGTTGGTCAAAAAGGAT
(SEQ ID NO: 62)
GROS_g00263: CCGCTCTGTGGATTCTTGGCGAATATTGCG

Transfection of Col-0 Protoplasts

As described above, Arabidopsis thaliana (Col-0) protoplasts were transfected with a vector coding for Crispr/Cas9 and sgRNAs and a vector containing the donor template to achieve HDR-mediated swaps. The experiment was designed such that sequences in the Tas1b (AtTAS1b_AT1G50055) or Tas3a (AtTAS3a_AT3G17185) genes were swapped, generating long-dsRNA and small secondary RNAs that target 30 bp sequences in the above-described nematode target genes. Two swaps were designed in the TAS1b locus, and two swaps in the TAS3a locus. Swaps were independent from each other.

The various combination of vectors used in the different experimental conditions is listed in Table 4 below. Different combinations of TAS backbones and donor oligos were used. Negative control transfections were carried out with no DNA (Treatment E).

TABLE 4
Experimental conditions

| | sgRNA Vector | |
|---|---|---|
| Exp. | (Crispr/Cas9, | |
| Condition | sgRNA, mCHERRY) | DONOR Vector |
| A | sgRNA_AtTAS1b | GEiGS-Y25-DONOR |
| B | sgRNA_AtTAS1b | GEiGS-Splicing |
| | | factor-DONOR |
| C | sgRNA_AtTAS3a | GEiGS- Ribosomal |
| | | protein 3a -DONOR |
| D | sgRNA_AtTAS3a | GEiGS-Spliceosomal |
| | | SR protein-DONOR |
| E | — | — |

Genomic Evidence of Tas1b and Tas3a Swaps in Col-0 Cells

Only a small fraction of transfected cells was expected to have successfully repaired DNA double strand breaks with an HDR Donor template, generating a swap. This is due to the low frequency of HDR events, as known in the art. Therefore, even the transfected samples were expected to contain a significant number of cells in which no swap took place.

In order to demonstrate that all the processed samples were suitable for PCR amplification, PCR reactions were carried out using WT specific primers on genomic DNA obtained from all treatments (A to E). The forward primer was designed to anneal to the region where swaps were intended to take place, while the reverse primer was designed to anneal further downstream the recombination site (FIG. 9A, primers denoted by arrows and the expected PCT product depicted as a dashed line). One primer set was designed for WT Tas1b and a different one for WT Tas3a. The expected PCR products (594 bp long) were obtained for WT Tas1b, Y25 and Splicing factor swap treatments (WT=Treatment E). In a similar way, the expected PCR products (574 bp long) were obtained for WT Tas3a, Ribosomal protein 3a and Spliceosome SR protein swap treatment. No amplification was obtained for negative controls, as expected (water, no template) (FIGS. 9B-C).

Specific PCR reactions were then carried out with the same unspecific reverse primer annealing further downstream the recombination site (one unspecific primer for WT Tas1b and a different one for WT Tas3a) and a swap-specific forward primer (FIG. 9A). As a control for the specificity of the PCR reaction WT DNA was used for a negative control for each primer pair (FIGS. 9E-F). The expected specific PCR products were obtained for Y25 (587 bp long) and Splicing factor (584 bp long) swap treatments for Tas1b. In a similar way, the expected specific PCR products were obtained for Ribosomal protein 3a (568 bp long) and Spliceosome SR protein (574 bp long) swap treatments (FIGS. 9D-E). No amplification was obtained when WT DNA was used as a template for all PCR reactions, further demonstrating the specificity of swap specific primers. Furthermore, no amplification was obtained for negative controls, as expected (water, no template).

Crude PCR products were further Sanger sequenced (Eurofins) using the unspecific reverse primer. Sequencing results were analysed using Snapgene software. It was expected to detect some mutations introduced by the HDR swaps (and not introduced by the primers used) right before the specific primer binding sites. Sequencing reactions confirmed the identity and location of such mutations. WT specific products were also sent for sequencing, both for Tas1b and Tas3a, following a similar approach and identity of WT sequences could also be confirmed (FIG. 9F). Results confirmed that sgRNA guides were active and HDR swaps took place for all treatments, both for Tas1b and Tas3a loci and using different donor oligos.

Similar results were obtained when following a nested PCR approach in which an unspecific PCR reaction was carried out before doing a nested, specific PCR reactions using the same sets of primers that were used for the main approach.

Genomic PCR

Cell samples (A, B, C, D, E) were processed for genomic DNA using a RNA/DNA Purification Kit (as discussed above).

As noted above, an unspecific primer flanking the swap region was used for the Tas1b (AtTAS1b_AT1G50055) and Tas3a (AtTAS3a_A T3G17185) sequences. As a negative control the same swap specific reactions were carried out using wild-type (WT) DNA as template. No amplification was expected. As a positive PCR control a specific PCR for WT DNA was carried out for all samples.

A similar alternative approach was also followed to confirm swaps. Instead of a single PCR reaction, a Nested PCR reaction was carried out. The first genomic PCR comprised unspecific forward and reverse primers flanking the HDR region. Unspecific primers used for the first PCR in the Nested approach have annealing sites flanking the annealing sited for the nested primers.

Genes and Sequences

Table 5 below lists (for each combination of TAS backbone and nematode target gene) the region of the GEiGS-oligo within the GEiGS donor, which includes the intended swaps, and will give rise to the siRNA that will target the gene in the nematode. The sequences of the wild-type TAS backbones, the sgRNAs used and the GEiGS donor designs are listed below.

Homologous regions in the GEiGS designs (i.e. regions which are intended to swap the wild type region in order to redirect the silencing activity and specificity of the TAS long dsRNA towards silencing of the target gene) are shown underlined. In the sequences below, the donor sequence inserted in the donor vector and containing the swapped nucleotides with homology arms is termed, for example, GEiGS-Splicing factor-DONOR. The long dsRNA transcripts of the TAS genes after the swap event, that will target the genes in the nematode, are termed, for example, GEiGS-Splicing factor-transcript.

TABLE 5
Swapped oligos

| | | | Homologous region in the | |
|---|---|---|---|---|
| GEiGS-oligo name | target gene | Backbone | GEiGS design | Amplifier |
| GEiGS-Splicing factor | Splicing factor | atTAS1b (e.g. | GTCGTTGAATTTCATTGATCT | miR173 |
| | | AtTAS1b_AT1 | GCTGCTCCA (SEQ ID NO: 76) | |
| | | G50055 - SEQ | | |
| | | ID NO: 73) | | |
| GEiGS-Spliceosomal | Spliceosomal SR | atTAS3a (e.g. | ATCCTTTTTGACCAACATTTG | miR390 |
| SR protein | protein | AtTAS3a_AT3 | TTTGTTTTT (SEQ ID NO: 90) | |
| | | G17185 - SEQ | | |
| | | ID NO: 83) | | |
| GEiGS-Y25 | Y25, beta subunit | atTAS1b (e.g. | CGCAATATTCGCCAAGAATC | miR173 |
| | of COPI complex | A1TAS1b_AT1 | CACAGAGCGG (SEQ ID NO: | |
| | | G50055 - SEQ | 80) | |
| | | ID NO: 73) | | |
| GEiGS-Ribosomal | Ribosomal protein | atTAS3a (e.g. | CGGCTTCTTCAGCACCTTCA | miR390 |
| protein 3a | 3a | AtITAS3a_AT3 | CCTTACGAAT (SEQ ID NO: | |
| | | G17185 - SEQ | 86) | |
| | | ID NO: 83) | | |

Additional Sequences per Table 5:
AtTAS1b_AT1G50055—SEQ ID NO: 73
sgRNA_AtTAS1b (including PAM)—SEQ ID NO: 74
GEiGS-Splicing factor-transcript—SEQ ID NO: 75
Homologous region in the GEiGS design of GEiGS-Splicing factor-transcript—SEQ ID NO: 76
GEiGS-Splicing factor-DONOR—SEQ ID NO: 77
Homologous region in the GEiGS design of GEiGS-Splicing factor-DONOR—SEQ ID NO: 78
GEiGS-Y25-transcript—SEQ ID NO: 79
Homologous region in the GEiGS design of GEiGS-Y25-transcript—SEQ ID NO: 80
Y25-DONOR—SEQ ID NO: 81
Homologous region in the GEiGS design of Y25-DONOR—SEQ ID NO: 82
AtTAS3a_AT3G17185—SEQ ID NO: 83
sgRNA_AtTAS3a (including PAM)—SEQ ID NO: 84
GEiGS-Ribosomal protein 3a-transcript—SEQ ID NO: 85
Homologous region in the GEiGS design of GEiGS-Ribosomal protein 3a-transcript—SEQ ID NO: 86
GEiGS-Ribosomal protein 3a -DONOR—SEQ ID NO: 87
Homologous region in the GEiGS design of GEiGS-Ribosomal protein 3a-DONOR—SEQ ID NO: 88
GEiGS-Spliceosomal SR protein-transcript—SEQ ID NO: 89
Homologous region in the GEiGS design of GEiGS-Spliceosomal SR protein-transcript—SEQ ID NO: 90
GEiGS-Spliceosomal SR protein-DONOR—SEQ ID NO: 91
Homologous region in the GEiGS design of GEiGS-Spliceosomal SR protein-DONOR—SEQ ID NO: 92

### Example 4

### Long Double-Stranded RNA in Cells Expressing a TAS Gene Modified by GEiGS

In order to demonstrate that modifying a nucleic acid sequence of a plant gene encoding a long dsRNA results in a modified dsRNA in the cell, RNA originating from the protoplasts analysed in Example 3 was used. RNA was reverse transcribed using specific primers to the target tested loci (on a region that was not designed to be swapped). Then, using primers specific to the swap region (to specifically amplify a swapped sequence or a wt sequence), the presence of long dsRNA, as a sense and anti-sense of the predicted RNA transcript, was studied.

RNA was extracted from all treatments and treated with DNAse to remove traces of DNA. RNA samples were then subjected to RT-PCR using an unspecific primer to generate cDNA. Two different independent RT-PCR (+RT) reactions were carried out to generate cDNA from the Sense and Anti-sense strand of Tas DNA, respectively. The approach was followed both for Tas1b and Tas3a. Reverse transcription controls (−RT) were carried out with all the same reagents but water was added instead of Reverse Transcriptase. If there was no reverse transcriptase in the reaction mix no cDNA was generated so any PCR products obtained in subsequent PCR reactions were necessarily amplified from carry-over DNA that remained intact after DNAse treatment of samples.

Specific PCR reactions were carried out with an unspecific forward primer and a swap-specific reverse primer (for the Sense cDNA) (FIG. 10A) or an unspecific reverse primer and a swap-specific forward primer (for the Antisense cDNA) (FIG. 10B). PCR reactions were designed in such a way that the length for all PCR products was lower than 200 nucleotides (FIGS. 10A-B).

WT specific PCR reactions for Tas1b (FIGS. 10C-D, right panels) and Tas3a sequences (FIGS. 10E-F, right panels) showed that RNA from treated samples was suitable for PCR amplification. Expected PCR products were obtained for the wt loci (Tas1b and Tas3a genes) both for Sense (105 bp for Tas1b, 133 bp for Tas3a) and Anti-sense strand (147 bp for Tas1b, 101 bp for Tas3a), in the treated samples, showing their coexistence and thus the presence of dsRNA in the samples. Clean −RT reactions indicated that traces of DNA were successfully removed from the RNA samples by DNAse treatment.

Swap specific RT-PCR reactions were carried out for treated RNA, and specific differentially amplified PCR products were obtained for Y25 Sense (98 bp) and Anti-sense treatments (149 bp) (FIGS. 10C-D, left panels). In the same manner specific differentially amplified PCR products were obtained for Ribosomal protein 3a Sense (130 bp) and Anti-sense treatments (118 bp) (FIGS. 10E-F, left panels). No amplification was obtained in negative controls using water and no RT template. As a negative control for each specific PCR reaction, the same master mixes were used for PCR using RNA from non treated cells as a template (treatment E). Lack of strong bands of the expected sizes showed the specificity for the swap specific primers, demonstrating RNA expression from the swapped loci.

Crude PCR products were Sanger sequenced using the unspecific forward primer in the case of the Sense approach (FIG. 10G) and the unspecific reverse primer in the case of the Anti-sense approach (FIG. 10H). It was expected to detect some mutations introduced by the HDR swaps (and not introduced by the printers used) right before the specific primer binding sites. Sequencing reactions confirmed the identity and location of such mutations for Tas1b Y25 swap and Tas3a Ribosomal protein 3a swaps (FIGS. 10G-H). WT specific products were also sent for sequencing, both for Tas1b and Tas3a, following a similar approach and identity of WT sequences could also be confirmed (FIG. 10I).

Thus, existence of dsRNA transctipts containing swaps was successfully proven within the cells treated using treatments A and C of Example 3 (i.e. a dsRNA of Tas1b containing swaps targeting it towards the Y25 target gene, and a dsRNA of Tas3a containing swaps targeting it towards the Ribosomal protein 3a target gene).

### Example 5

Silencing Activity of Long-dsRNA with Altered Targeting Specificity in Nicotiana Benthamiana

The following experiment demonstrated silencing activity towards a target gene of choice when using dsRNA in which targeting specificity (of small RNAs processed from it) has been redirected towards the gene of choice (e.g. using the GEiGS approach of HDR-mediated redirection of silencing specificity). To do so, a transient expression system was used through infiltration of Nicotiana benthamiana leaves with: (1) a Turnip mosaic virus (TuMV) vector with GFP marker, and (2) a vector for overexpression of the “GEiGS design”—a TAS gene encoding for a transcript based on TAS1b with nucleotide changes necessary for targeting TuMV, which could be generated by using GEiGS to introduce the nucleotide changes into the TAS1b gene backbone in the Arabidopsis genome (also referred to below as “GEiGS-TuMV”). Infiltration was carried out by introducing agrobacterium bacteria of strain GV3101, which have been transformed with the various vectors, into the leaves.

The oligonucleotides which are required to generate “GEiGS design” using the GEiGS approach, as described above for A. thaliana, and in particular—(1) the sgRNA which are used to cut TAS1b, (2) the siRNA sequence that targets TuMV (which are introduced into the TAS1b backbone by the GEiGS donor using an HDR-mediated swap), (3) the GEiGS donor which includes the desired changes to the TAS1b backbone, are as follows:

(1) The sgRNA which would have been used to cut TAS1b SEQ ID NO: 134

(2) The siRNA sequence that targets TuMV (which would have been introduced into the TAS1b backbone by the GEiGS donor using an HDR-mediated swap)—SEQ ID NO: 135

(3) The GEiGS donor which included the desired changes to the TAS1b backbone—SEQ ID NO: 136

(4) The GEiGS oligo Which would have been expressed in Arabidopsis following GEiGS with the donor of (3) (designed to introduce the mature siRNA sequence of (1) into the TAS1b sequence)—SEQ ID NO: 137.

The incorporation of a fluorescent GFP reporter gene into a replication component of the TuMV enabled to monitor the growth and spread of the TuMV in the leaf and thus the silencing efficacy of TuMV specific silencing molecules on the virus.

The amplifier for generating RdRp-dependent transcription of TAS1b is miR173. Therefore, the TuMV-GFP vector was co-infiltrated with/without the miR173 amplifier, expecting to see silencing activity of the TuMV-targeting dsRNA when the amplifier is present.

As a negative control, a vector for overexpressing a “GEiGS design” with no specific known target (also referred to as “dummy” or “GEiGS-Dummy”) was infiltrated into leaves e. dsRNA based on TAS1b with nucleotide changes in locations corresponding to those changed in the “GEiGS-TuMV” but which do not correspond with any known gene in Nicotiana benthamiana). Both the vectors expressing the dummy control or the “GEiGS-TuMV” dsRNA were infiltrated into the leaves with or without the amplifier. In order to maintain the level of infiltrated inoculums constant between treatments, empty agrobacterium were used in treatments without certain components (see Table 6, below).

TABLE 6
N. benthamiana leaf infiltration (side by side assay)

| | Left side | | | Right side | | |
|---|---|---|---|---|---|---|
| | Vector #1 | Vector #2 | Vector #3 | Vector #1 | Vector #2 | Vector #3 |
| 1 | No vector | No vector | TuMV-GFP | No vector | No vector | No vector |
| 2 | No vector | miR173 | TuMV-GFP | No vector | No vector | TuMV-GFP |
| 3 | GEiGS-Dummy | No vector | TuMV-GFP | GEiGS-TuMV | No vector | TuMV-GFP |
| 4 | GEiGS-Dummy | miR173 | TuMV-GFP | GEiGS-TuMV | miR173 | TuMV-GFP |

As can be seen in FIG. 11A, two different treatments were infiltrated into each leaf, side by side, measuring the GFP level (corresponding to TuMV level) in each side of the leaf (the observations have been further confirmed by a qRT-PCR analysis). Each treatment was repeated at least 3 times, observed under UV light, and one was sacrificed for photography.

In one leaf (leaf 1), a vector expressing (+TuMV) was infiltrated, comparing the GFP levels with a treatment in which TuMV was not infiltrated (−TuMV). As expected, there was no background fluorescence when no virus was present. In a second leaf (leaf 2), the TuMV-GFP virus was infiltrated with the amplifier, miR173 (+miR173), or without (−miR173), demonstrating that miR173 by itself had no effect on the replication of the virus (as it did not have a significant effect on the measurement of relative expression by qRT-PCT). In a third leaf (leaf 3) and a fourth leaf (leaf 4), the vector expressing the TuMV virus was infiltrated with a construct expressing either dsRNA not targeting a known gene (GEiGS-Dummy), or with the dsRNA altered such that it targets the virus (GEiGS-TuMV). This was done either without the amplifier (leaf 3) or with (leaf 4).

In the presence of the amplifier (leaf 4), a clear significant reduction in TuMV transcript, compared to the dummy treatment, as well as a visual GFP signal reduction, was observed, as noted by the relative expression in FIG. 11A. The slight decrease in GFP level observed in leaf 3 when infiltrating the GEiGS-TuMV construct (without the amplifier) was determined by a qRT-PCR analysis to be too variable to be considered significant.

Infiltration of whole leaves (FIG. 11B) has been carried out using the system described above, by infiltrating the vector expressing the TuMV-GFP fusion, the amplifier and a vector expressing a dsRNA construct (either the “GEiGS-TuMV”, targeting TuMV or the “GEiGS-Dummy”, not targeting a known gene, see Table 7 below). As a control, a leaf infiltrated by agrobacterium with no vector was used. A clear reduction of GFP levels was observed when using the GEiGS-TuMV dsRNA but not the GEiGS-Dummy. This emphasised the effect of the GEiGS design and amplifier gene on TuMV replication.

TABLE 7
N. benthamiana leaf infiltration (whole leaf assay)

| | Vector #1 | Vector #2 | Vector #3 |
|---|---|---|---|
| 1 | No vector | No vector | No vector |
| 2 | GEiGS-Dummy | miR173 | TuMV-GFP |
| 3 | GEiGS-TuMV | miR173 | TuMV-GFP |

These results confirmed the role of the TAS gene and the amplifier in inducing silencing, as expected from the accepted model of an amplifier-dependent tali-RNA pathway. The results further confirmed the feasibility of expressing a dsRNA altered using GEiGS in a plant in order to silence gene expression of a target of choice in a pest (e.g. by introducing desired nucleotide changes into a gene encoding the dsRNA, thus redirecting the dsRNA to silence a target of choice).

### Example 6

### Expression of Long-dsRNA Targeting a Nematode Gene In-Planta Induces Silencing of its Target Gene in Nematode

This experiment was intended to demonstrate that a silencing dsRNA molecule (such as that expressed from a TAS gene), which is expressed in a plant and which has been redirected to target a pest gene (e.g. a nematode gene) can induce silencing of its target gene in the pest (e.g. nematode).

In order to so, a transient expression system was used to express the tested dsRNA molecules in Nicotiana benthamiana leaves, introducing them into the leaves by agrobacterium-mediated infiltration to the leaves, as described above. Then nematodes were fed with a leaf extraction, as described below, and the effect on target gene expression was examined.

The analysis has been carried out through targeting the Ribosomal protein 3a and the Spliceosomal SR protein genes in the nematode Globodera rostochiensis. In particular, Nicotiana benthamiana leaves were infiltrated with agrobacterium containing a vector that overexpressed a TAS3a transcript into which nucleotide changes have been introduced. The nucleotide changes at least partially redirected the silencing specificity of the TAS3a transcript towards one of these nematode genes. Corresponding changes could be introduced into the TAS3a gene in a plant cell using Gene Editing induced Gene Silencing (GEiGS), by inducing a DNA break in the gene (e,g. using an endonuclease such as Cas9 and a specific sgRNA) and introducing the changes into the gene via Homology Dependent Recombination (HDR) with a GEiGS-donor oligonucleotide that contained the desired nucleotide changes. Sequences of a GEiGS oligo and a sgRNA sequence that may be used to introduce specificity against Ribosomal protein 3a into the TAS3a gene are provided in Example 3 above.

As a control, leaves were also infiltrated with a wild-type transcript of TAS3a. Both leaves infiltrated by the control TAS3a and the TAS3a modified to target the nematode genes were further infiltrated with the amplifier miR390.

After 48 and 72 hours, leaves were collected, and total RNA was extracted and cleaned on Amicon® Ultra 0.5 mL Centrifugal Filters 3KD cut-off (Merck, USA). Globodera rostochiensis Nematodes were fed with this total RNA for 72 hours as described below and collected. RNA was extracted and gene expression analysis was carried out with qRT-PCR, using Actin as an endogenous normaliser gene. Ribosomal Protein 3a (FIG. 12A) and Spliceosomal SR protein (FIG. 12B), both, have shown to be substantially reduced in their expression levels in the in-planta fed nematode tests. The expression of Ribosomal protein 3a was shown to be reduced with a T-test significance of 7×10−5 and the expression of Spiceosomal SR protein with a T-test significance of 1.72×10−3, indicating the targeted genes have been significantly silenced and should show reduction in nematode growth in the following generation.

These results demonstrate that modifications made on Tas3a, that led to the formation of dsRNA which targets nematode genes, can target pathogens that are sensitive to such a dsRNA.

RNA extract that was used for the feeding of nematodes, was also analysed through RNA-seq and small RNA-seq (Cambridge Genomic Services, Cambridge, UK; FIGS. 13A-D). Analysis was carried out as described in methods. Sequence reads were aligned against the sequence of the GEiGS designs that aimed to target ribosomal protein 3a (FIGS. 13A and 13B), and spliceosomal SR protein (FIGS. 13C and 13D). Alignment was carried out on both strands, sense and antisense. Analyses have confirmed the presence of both strands of the transcript, with the capability of generating a long double stranded RNA through analysis of long RNAseq reads (FIGS. 13A and 13C) and short small RNAseq reads (FIGS. 13B and 13D). Since RNA-seq analysis has been carried out using reads longer than 50 nucleotides, this analysis identified long double stranded RNA. In addition, the small RNA analysis was carried out through filtering sequences of 20 to 24 nucleotides, thus demonstrating the phased processing of the long-dsRNA, confirming the formation of the secondary siRNA (FIGS. 13B and 13D).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### REFERENCES

Yadav, B., Veluthambi, K. and Subramaniam, K. (2006). Host-generated double stranded RNA induces RNAi in plant-parasitic nematodes and protects the host from infection. Molecular and Biochemical Parasitology, 148(2), pp. 219-222.

Klink, V., Kim, K., Martins, V., MacDonald, M., Beard, H., Alkharouf, N., Lee, S., Park, S. and Matthews, B. (2009). A correlation between host-mediated expression of parasite genes as tandem inverted repeats and abrogation of development of female Heterodera glycines cyst formation during infection of Glycine max. Planta, 230(1), pp. 53-71.

Li, J., Todd, T., Oakley, T., Lee, J. and Trick, H. (2010). Host-derived suppression of nematode reproductive and fitness genes decreases fecundity of Heterodera glycines Ichinohe. Planta, 232(3), pp. 775-785.

Li, J., Todd, T. and Trick, H. (2009). Rapid in planta evaluation of root expressed transgenes in chimeric soybean plants. Plant Cell Reports, 29(2), pp. 113-123.

What is claimed is:

1. A method of producing a long dsRNA molecule in a plant cell that is capable of silencing a pest gene, the method comprising:
(a) selecting in a genome of a plant a nucleic acid sequence encoding a silencing molecule having a plant gene as a target, said silencing molecule capable of recruiting RNA-dependent RNA Polymerase (RdRp);
(b) modifying a nucleic acid sequence of the plant gene so as to impart a silencing specificity towards the pest gene, such that a transcript of said plant gene comprising said silencing specificity forms base complementation with said silencing molecule capable of recruiting said RdRp to produce the long dsRNA molecule capable of silencing the pest gene,
thereby producing the long dsRNA molecule in the plant cell that is capable of silencing the pest gene.

2. The method of claim 1, wherein said silencing molecule capable of recruiting said RdRp comprises 21-24 nucleotides.

3. The method of any one of claims 1-2, wherein said silencing molecule capable of recruiting said RdRp is selected from the group consisting of: trans-acting siRNA (tasiRNA), phased small interfering RNA (phasiRNA), microRNA (miRNA), small interfering RNA (siRNA), short hairpin RNA (shRNA), Piwi-interacting RNA (piRNA), transfer RNA (tRNA), small nuclear RNA (snRNA), ribosomal RNA (rRNA), small nucleolar RNA (snoRNA), extracellular RNA (exRNA), repeat-derived RNA, autonomous and non-autonomous transposable RNA.

4. The method of claim 3, wherein said miRNA comprises a 22 nucleotides mature small RNA,

5. The method of claim 3 or 4, wherein said miRNA is selected from the group consisting of: miR-156a, miR-156c, miR-162a, miR-162b, miR-167d, miR-169b, miR-173, miR-393a, miR-393b, miR-402, miR-403, miR-447a, miR-447b, miR-447c, miR-472, miR-771, miR-777, miR-828, miR-830, miR-831, miR-833a, miR-833a, miR-845b, miR-848, miR-850, miR-853, miR-855, miR-856, miR-864, miR-2933a, miR-2933b, miR-2936, miR-4221, miR-5024, miR-5629, miR-5648, miR-5996, miR-8166, miR-8167a, miR-8167b, miR-8167c, miR-8167d, miR-8167e, miR-8167f, miR-8177, and miR-8182.

6. The method of any one of claims 1-5, wherein said plant gene is a non-protein coding gene.

7. The method of any one of claims 1-6, wherein the plant gene encodes for a molecule having an intrinsic silencing activity towards a native plant gene.

8. The method of any one of claims 1-7, wherein said modifying of step (b) comprises introducing into the plant cell a DNA editing agent which redirects a silencing specificity of said plant gene towards said pest gene, said pest gene and a native plant gene being distinct.

9. The method of claim 7 or 8, wherein said plant gene having said intrinsic silencing activity is selected from the group consisting of trans-acting siRNA (tasiRNA), phased small interfering RNA (phasiRNA), microRNA (miRNA), small interfering RNA (siRNA), short hairpin RNA (shRNA), Piwi-interacting RNA (piRNA), transfer RNA (tRNA), small nuclear RNA (snRNA), ribosomal RNA (rRNA), small nucleolar RNA (snoRNA), extracellular RNA (exRNA), autonomous and non-autonomous transposable RNA.

10. The method of any one of claims 7-9, wherein said plant gene having said intrinsic silencing activity encodes for a phased secondary siRNA-producing molecule.

11. The method of any one of claims 7-9, wherein said plant gene having said intrinsic silencing activity is a trans-acting-siRNA-producing (TAS) molecule.

12. The method of any one of claims 1-11, wherein said silencing specificity of said plant gene is determined by measuring a transcript level of said pest gene.

13. The method of any one of claims 1-12, wherein said silencing specificity of said plant gene is determined phenotypically.

14. The method of claim 13, wherein said determined phenotypically is effected by determination of pest resistance of said plant.

15. The method of any one of claims 1-14, wherein said silencing specificity of said plant gene is determined genotypically.

16. The method of claim 15, wherein a plant phenotype is determined prior to a plant genotype.

17. The method of claim 15, wherein a plant genotype is determined prior to a plant phenotype.

18. A method of producing a long dsRNA molecule in a plant cell that is capable of silencing a pest gene, the method comprising:
(a) selecting a nucleic acid sequence of a plant gene exhibiting a predetermined sequence homology to a nucleic acid sequence of the pest gene;
(b) modifying a plant endogenous nucleic acid sequence encoding an RNA molecule so as to impart silencing specificity towards said plant gene, such that small RNA molecules capable of recruiting RNA-dependent RNA Polymerase (RdRp) processed from said RNA molecule form base complementation with a transcript of said plant gene to produce the long dsRNA molecule capable of silencing the pest gene,
thereby producing the long dsRNA molecule in the plant cell that is capable of silencing the pest gene.

19. The method of claim 18, wherein said predetermined sequence homology comprises 75-100% identity.

20. The method of any one of claims 18-19, wherein said small RNA molecules capable of recruiting said RdRp comprise 21-24 nucleotides.

21. The method of any one of claims 18-20, wherein said small RNA molecules capable of recruiting said RdRp are selected from the group consisting of microRNA (miRNA), small interfering RNA (siRNA), short hairpin RNA (shRNA), kiwi-interacting RNA (piRN), trans-acting siRNA (tasiRNA), phased small interfering RNA (phasiRNA), transfer RNA (tRNA), small nuclear RNA (snRNA), ribosomal RNA (rRNA), small nucleolar RNA (snoRNA), extracellular RNA (exRNA), repeat-derived RNA, autonomous and non-autonomous transposable RNA.

22. The method of any one of claims 18-21, wherein said RNA molecule has an intrinsic silencing activity towards a native plant gene.

23. The method of any one of claims 18-22, wherein said modifying of step (b) comprises introducing into the plant cell a DNA editing agent which redirects a silencing specificity of said RNA molecule towards said plant gene, said plant gene and a native plant gene being distinct.

24. The method of any one of claims 18-23, wherein said plant gene exhibiting said predetermined sequence homology to said nucleic acid sequence of the pest gene does not encode a silencing molecule.

25. The method of any one of claims 18-24, wherein said silencing specificity of said RNA molecule is determined by measuring a transcript level of said plant gene or said pest gene.

26. The method of any one of claims 18-25, wherein said silencing specificity of the RNA molecule is determined phenotypically.

27. The method of claim 26, wherein said determined phenotypically is effected by determination of pest resistance of said plant.

28. The method of any one of claims 18-27, wherein said silencing specificity of the RNA molecule is determined genotypically.

29. The method of claim 28, wherein a plant phenotype is determined prior to a plant genotype.

30. The method of claim 28, wherein a plant genotype is determined prior to a plant phenotype.

31. The method of any one of claim 8-17 or 23-30, wherein said DNA editing agent comprises at least one sgRNA.

32. The method of any one of claim 8-17 or 23-31, wherein said DNA editing agent does not comprise an endonuclease.

33. The method of any one of claim 8-17 or 23-31, wherein said DNA editing agent comprises an endonuclease.

34. The method of any one of claims 8-17 or 23-33, wherein said DNA editing agent is of a DNA editing system selected from the group consisting of a meganuclease, a zinc finger nucleases (ZFN), a transcription-activator like effector nuclease (TALEN), CRISPR-endonuclease, dCRISPR-endonuclease and a homing endonuclease.

35. The method of claim 33 or 34, wherein said endonuclease comprises Cas9.

36. The method of any one of claim 8-17 or 23-35, wherein said DNA editing agent is applied to the cell as DNA, RNA or RNP.

37. The method of any one of claims 1-36, wherein said plant cell is a protoplast.

38. The method of any one of claims 1-37, wherein a dsRNA molecule is processable by cellular RNAi processing machinery.

39. The method of any one of claims 1-38, wherein a dsRNA molecule is processed into secondary small RNAs.

40. The method of any one of claims 1-39, wherein said dsRNA and/or said secondary small RNAs comprise a silencing specificity towards a pest gene.

41. A method of generating a pest tolerant or resistant plant, the method comprising producing a long dsRNA molecule in a plant cell capable of silencing a pest gene according to any one of claims 1-40.

42. The method of claim 41, wherein said pest is an invertebrate.

43. The method of claim 41 or 42, wherein said pest is selected from the group consisting of a virus, an ant, a termite, a bee, a wasp, a caterpillar, a cricket, a locust, a beetle, a snail, a slug, a nematode, a bug, a fly, a fruitfly, a whitefly, a mosquito, a grasshopper, a planthopper, an earwig, an aphid, a scale, a thrip, a spider, a mite, a psyllid, a tick, a moth, a worm, a scorpion and a fungus.

44. A plant generated by the method of any one of claims 1-43.

45. The plant of claim 44, wherein the plant is selected from the group consisting of a crop, a flower, a weed, and a tree.

46. The plant of claim 44 or 45, wherein said plant is non-transgenic.

47. A cell of the plant of any one of claims 44-46.

48. A seed of the plant of any one of claims 44-46.

49. A method of producing a pest tolerant or resistant plant, the method comprising:
(a) breeding the plant of any one of claims 44-46; and
(b) selecting for progeny plants that express the long dsRNA molecule capable of suppressing the pest gene, and which do not comprise said DNA editing agent,
thereby producing said pest tolerant or resistant plant.

50. A method producing a plant or plant cell of any one of claims 44-47 comprising growing the plant or plant cell under conditions which allow propagation.
